SEQUENCE LISTING (284 sequences)

SEQ ID NO: 1 - protein, 9 aa, Artificial Sequence
  peptide
ALGCLYTFV

SEQ ID NO: 2 - protein, 9 aa, Artificial Sequence
  peptide
AMGCLYTFI

SEQ ID NO: 3 - protein, 9 aa, Artificial Sequence
  peptide
AMGCLYTFV

SEQ ID NO: 4 - protein, 9 aa, Artificial Sequence
  peptide
CMYTFLISV

SEQ ID NO: 5 - protein, 9 aa, Artificial Sequence
  peptide
CSDDGIWSI

SEQ ID NO: 6 - protein, 9 aa, Artificial Sequence
  peptide
CSDDGIWSL

SEQ ID NO: 7 - protein, 9 aa, Artificial Sequence
  peptide
CSDDGIWSV

SEQ ID NO: 8 - protein, 9 aa, Artificial Sequence
  peptide
FASDYKDFY

SEQ ID NO: 9 - protein, 9 aa, Artificial Sequence
  peptide
FIGCLYTFI

SEQ ID NO: 10 - protein, 9 aa, Artificial Sequence
  peptide
FIGCLYTFL

SEQ ID NO: 11 - protein, 9 aa, Artificial Sequence
  peptide
FIGCLYTFV

SEQ ID NO: 12 - protein, 9 aa, Artificial Sequence
  peptide
FIVKPLPPV

SEQ ID NO: 13 - protein, 9 aa, Artificial Sequence
  peptide
FLCSWKPGI

SEQ ID NO: 14 - protein, 9 aa, Artificial Sequence
  peptide
FLCSWKPGL

SEQ ID NO: 15 - protein, 9 aa, Artificial Sequence
  peptide
FLCSWKPGV

SEQ ID NO: 16 - protein, 9 aa, Artificial Sequence
  peptide
FLDDGIWSI

SEQ ID NO: 17 - protein, 9 aa, Artificial Sequence
  peptide
FLDDGIWSL

SEQ ID NO: 18 - protein, 9 aa, Artificial Sequence
  peptide
FLDDGIWSV

SEQ ID NO: 19 - protein, 9 aa, Artificial Sequence
  peptide
FLDHALQCV

SEQ ID NO: 20 - protein, 9 aa, Artificial Sequence
  peptide
FLDKQCWEV

SEQ ID NO: 21 - protein, 9 aa, Artificial Sequence
  peptide
FLFYWYEGI

SEQ ID NO: 22 - protein, 9 aa, Artificial Sequence
  peptide
FLFYWYEGL

SEQ ID NO: 23 - protein, 9 aa, Artificial Sequence
  peptide
FLFYWYEGV

SEQ ID NO: 24 - protein, 9 aa, Artificial Sequence
  peptide
FLGCLYTFI

SEQ ID NO: 25 - protein, 9 aa, Artificial Sequence
  peptide
FLGCLYTFL

SEQ ID NO: 26 - protein, 9 aa, Artificial Sequence
  peptide
FLGCLYTFV

SEQ ID NO: 27 - protein, 9 aa, Artificial Sequence
  peptide
FLISTTFGI

SEQ ID NO: 28 - protein, 9 aa, Artificial Sequence
  peptide
FLISTTFGL

SEQ ID NO: 29 - protein, 9 aa, Artificial Sequence
  peptide
FLISTTFGV

SEQ ID NO: 30 - protein, 9 aa, Artificial Sequence
  peptide
FLNETYTLK

SEQ ID NO: 31 - protein, 9 aa, Artificial Sequence
  peptide
FLPFGFILV

SEQ ID NO: 32 - protein, 9 aa, Artificial Sequence
  peptide
FLSDYKDFY

SEQ ID NO: 33 - protein, 9 aa, Artificial Sequence
  peptide
FLTNYNLFY

SEQ ID NO: 34 - protein, 9 aa, Artificial Sequence
  peptide
FLVKPLPPI

SEQ ID NO: 35 - protein, 9 aa, Artificial Sequence
  peptide
FLVKPLPPL

SEQ ID NO: 36 - protein, 9 aa, Artificial Sequence
  peptide
FLVKPLPPV

SEQ ID NO: 37 - protein, 9 aa, Artificial Sequence
  peptide
FLWKTIITK

SEQ ID NO: 38 - protein, 9 aa, Artificial Sequence
  peptide
FLWQPPLSI

SEQ ID NO: 39 - protein, 9 aa, Artificial Sequence
  peptide
FLWQPPLSL

SEQ ID NO: 40 - protein, 9 aa, Artificial Sequence
  peptide
FLWQPPLSV

SEQ ID NO: 41 - protein, 9 aa, Artificial Sequence
  peptide
FLYLLCSWK

SEQ ID NO: 42 - protein, 9 aa, Artificial Sequence
  peptide
FLYTFLISI

SEQ ID NO: 43 - protein, 9 aa, Artificial Sequence
  peptide
FLYTFLISL

SEQ ID NO: 44 - protein, 9 aa, Artificial Sequence
  peptide
FLYTFLIST

SEQ ID NO: 45 - protein, 9 aa, Artificial Sequence
  peptide
FLYTFLISV

SEQ ID NO: 46 - protein, 9 aa, Artificial Sequence
  peptide
FMCSWKPGI

SEQ ID NO: 47 - protein, 9 aa, Artificial Sequence
  peptide
FMCSWKPGL

SEQ ID NO: 48 - protein, 9 aa, Artificial Sequence
  peptide
FMCSWKPGV

SEQ ID NO: 49 - protein, 9 aa, Artificial Sequence
  peptide
FMDDGIWSI

SEQ ID NO: 50 - protein, 9 aa, Artificial Sequence
  peptide
FMDDGIWSL

SEQ ID NO: 51 - protein, 9 aa, Artificial Sequence
  peptide
FMDDGIWSV

SEQ ID NO: 52 - protein, 9 aa, Artificial Sequence
  peptide
FMDHALQCV

SEQ ID NO: 53 - protein, 9 aa, Artificial Sequence
  peptide
FMDKQCWEV

SEQ ID NO: 54 - protein, 9 aa, Artificial Sequence
  peptide
FMFYWYEGI

SEQ ID NO: 55 - protein, 9 aa, Artificial Sequence
  peptide
FMFYWYEGL

SEQ ID NO: 56 - protein, 9 aa, Artificial Sequence
  peptide
FMFYWYEGV

SEQ ID NO: 57 - protein, 9 aa, Artificial Sequence
  peptide
FMGCLYTFI

SEQ ID NO: 58 - protein, 9 aa, Artificial Sequence
  peptide
FMGCLYTFL

SEQ ID NO: 59 - protein, 9 aa, Artificial Sequence
  peptide
FMGCLYTFV

SEQ ID NO: 60 - protein, 9 aa, Artificial Sequence
  peptide
FMISTTFGI

SEQ ID NO: 61 - protein, 9 aa, Artificial Sequence
  peptide
FMISTTFGL

SEQ ID NO: 62 - protein, 9 aa, Artificial Sequence
  peptide
FMISTTFGV

SEQ ID NO: 63 - protein, 9 aa, Artificial Sequence
  peptide
FMNETYTLK

SEQ ID NO: 64 - protein, 9 aa, Artificial Sequence
  peptide
FMPFGFILV

SEQ ID NO: 65 - protein, 9 aa, Artificial Sequence
  peptide
FMQDMCVYY

SEQ ID NO: 66 - protein, 9 aa, Artificial Sequence
  peptide
FMSDYKDFY

SEQ ID NO: 67 - protein, 9 aa, Artificial Sequence
  peptide
FMTGLLLRK

SEQ ID NO: 68 - protein, 9 aa, Artificial Sequence
  peptide
FMTNYNLFY

SEQ ID NO: 69 - protein, 9 aa, Artificial Sequence
  peptide
FMVIFVTGV

SEQ ID NO: 70 - protein, 9 aa, Artificial Sequence
  peptide
FMVKPLPPI

SEQ ID NO: 71 - protein, 9 aa, Artificial Sequence
  peptide
FMVKPLPPL

SEQ ID NO: 72 - protein, 9 aa, Artificial Sequence
  peptide
FMVKPLPPV

SEQ ID NO: 73 - protein, 9 aa, Artificial Sequence
  peptide
FMWKTIITK

SEQ ID NO: 74 - protein, 9 aa, Artificial Sequence
  peptide
FMWQPPLSI

SEQ ID NO: 75 - protein, 9 aa, Artificial Sequence
  peptide
FMWQPPLSL

SEQ ID NO: 76 - protein, 9 aa, Artificial Sequence
  peptide
FMWQPPLSV

SEQ ID NO: 77 - protein, 9 aa, Artificial Sequence
  peptide
FMYLLCSWK

SEQ ID NO: 78 - protein, 9 aa, Artificial Sequence
  peptide
FMYTFLISI

SEQ ID NO: 79 - protein, 9 aa, Artificial Sequence
  peptide
FMYTFLISL

SEQ ID NO: 80 - protein, 9 aa, Artificial Sequence
  peptide
FMYTFLIST

SEQ ID NO: 81 - protein, 9 aa, Artificial Sequence
  peptide
FMYTFLISV

SEQ ID NO: 82 - protein, 9 aa, Artificial Sequence
  peptide
FQWQPPLSV

SEQ ID NO: 83 - protein, 9 aa, Artificial Sequence
  peptide
FSDDGIWSI

SEQ ID NO: 84 - protein, 9 aa, Artificial Sequence
  peptide
FSDDGIWSL

SEQ ID NO: 85 - protein, 9 aa, Artificial Sequence
  peptide
FSDDGIWSV

SEQ ID NO: 86 - protein, 9 aa, Artificial Sequence
  peptide
FSDKQCWEI

SEQ ID NO: 87 - protein, 9 aa, Artificial Sequence
  peptide
FSDKQCWEL

SEQ ID NO: 88 - protein, 9 aa, Artificial Sequence
  peptide
FSDKQCWEV

SEQ ID NO: 89 - protein, 9 aa, Artificial Sequence
  peptide
FSDYKDFYI

SEQ ID NO: 90 - protein, 9 aa, Artificial Sequence
  peptide
FSDYKDFYL

SEQ ID NO: 91 - protein, 9 aa, Artificial Sequence
  peptide
FSDYKDFYV

SEQ ID NO: 92 - protein, 9 aa, Artificial Sequence
  peptide
FSSWAETTY

SEQ ID NO: 93 - protein, 9 aa, Artificial Sequence
  peptide
FTWKTIITK

SEQ ID NO: 94 - protein, 9 aa, Artificial Sequence
  peptide
FVQDMCVYY

SEQ ID NO: 95 - protein, 9 aa, Artificial Sequence
  peptide
LLTNYNLFY

SEQ ID NO: 96 - protein, 9 aa, Artificial Sequence
  peptide
LLWQPPLSV

SEQ ID NO: 97 - protein, 9 aa, Artificial Sequence
  peptide
LMTNYNLFY

SEQ ID NO: 98 - protein, 9 aa, Artificial Sequence
  peptide
LMWQPPLSI

SEQ ID NO: 99 - protein, 9 aa, Artificial Sequence
  peptide
LMWQPPLSL

SEQ ID NO: 100 - protein, 9 aa, Artificial Sequence
  peptide
LMWQPPLSV

SEQ ID NO: 101 - protein, 9 aa, Artificial Sequence
  peptide
RLIGSETWK

SEQ ID NO: 102 - protein, 9 aa, Artificial Sequence
  peptide
RMIGSETWK

SEQ ID NO: 103 - protein, 9 aa, Artificial Sequence
  peptide
VLNETYTLK

SEQ ID NO: 104 - protein, 9 aa, Artificial Sequence
  peptide
VMNETYTLK

SEQ ID NO: 105 - protein, 9 aa, Artificial Sequence
  peptide
WASDYKDFY

SEQ ID NO: 106 - protein, 9 aa, Artificial Sequence
  peptide
WLCSWKPGV

SEQ ID NO: 107 - protein, 9 aa, Artificial Sequence
  peptide
WLDDGIWSI

SEQ ID NO: 108 - protein, 9 aa, Artificial Sequence
  peptide
WLDDGIWSL

SEQ ID NO: 109 - protein, 9 aa, Artificial Sequence
  peptide
WLDDGIWSV

SEQ ID NO: 110 - protein, 9 aa, Artificial Sequence
  peptide
WLFYWYEGV

SEQ ID NO: 111 - protein, 9 aa, Artificial Sequence
  peptide
WLGCLYTFI

SEQ ID NO: 112 - protein, 9 aa, Artificial Sequence
  peptide
WLGCLYTFL

SEQ ID NO: 113 - protein, 9 aa, Artificial Sequence
  peptide
WLGCLYTFV

SEQ ID NO: 114 - protein, 9 aa, Artificial Sequence
  peptide
WLISTTFGL

SEQ ID NO: 115 - protein, 9 aa, Artificial Sequence
  peptide
WLISTTFGV

SEQ ID NO: 116 - protein, 9 aa, Artificial Sequence
  peptide
WLNETYTLK

SEQ ID NO: 117 - protein, 9 aa, Artificial Sequence
  peptide
WLSDYKDFY

SEQ ID NO: 118 - protein, 9 aa, Artificial Sequence
  peptide
WLTNYNLFY

SEQ ID NO: 119 - protein, 9 aa, Artificial Sequence
  peptide
WLVKPLPPV

SEQ ID NO: 120 - protein, 9 aa, Artificial Sequence
  peptide
WLWQPPLSI

SEQ ID NO: 121 - protein, 9 aa, Artificial Sequence
  peptide
WLWQPPLSL

SEQ ID NO: 122 - protein, 9 aa, Artificial Sequence
  peptide
WLWQPPLSV

SEQ ID NO: 123 - protein, 9 aa, Artificial Sequence
  peptide
WLYTFLISV

SEQ ID NO: 124 - protein, 9 aa, Artificial Sequence
  peptide
WMCSWKPGV

SEQ ID NO: 125 - protein, 9 aa, Artificial Sequence
  peptide
WMDDGIWSI

SEQ ID NO: 126 - protein, 9 aa, Artificial Sequence
  peptide
WMDDGIWSL

SEQ ID NO: 127 - protein, 9 aa, Artificial Sequence
  peptide
WMDDGIWSV

SEQ ID NO: 128 - protein, 9 aa, Artificial Sequence
  peptide
WMDKQCWEV

SEQ ID NO: 129 - protein, 9 aa, Artificial Sequence
  peptide
WMFYWYEGV

SEQ ID NO: 130 - protein, 9 aa, Artificial Sequence
  peptide
WMGCLYTFI

SEQ ID NO: 131 - protein, 9 aa, Artificial Sequence
  peptide
WMGCLYTFL

SEQ ID NO: 132 - protein, 9 aa, Artificial Sequence
  peptide
WMGCLYTFV

SEQ ID NO: 133 - protein, 9 aa, Artificial Sequence
  peptide
WMISTTFGI

SEQ ID NO: 134 - protein, 9 aa, Artificial Sequence
  peptide
WMISTTFGL

SEQ ID NO: 135 - protein, 9 aa, Artificial Sequence
  peptide
WMISTTFGV

SEQ ID NO: 136 - protein, 9 aa, Artificial Sequence
  peptide
WMNETYTLK

SEQ ID NO: 137 - protein, 9 aa, Artificial Sequence
  peptide
WMSDYKDFY

SEQ ID NO: 138 - protein, 9 aa, Artificial Sequence
  peptide
WMTNYNLFY

SEQ ID NO: 139 - protein, 9 aa, Artificial Sequence
  peptide
WMVKPLPPV

SEQ ID NO: 140 - protein, 9 aa, Artificial Sequence
  peptide
WMWKTIITK

SEQ ID NO: 141 - protein, 9 aa, Artificial Sequence
  peptide
WMWQPPLSI

SEQ ID NO: 142 - protein, 9 aa, Artificial Sequence
  peptide
WMWQPPLSL

SEQ ID NO: 143 - protein, 9 aa, Artificial Sequence
  peptide
WMWQPPLSV

SEQ ID NO: 144 - protein, 9 aa, Artificial Sequence
  peptide
WMYTFLISI

SEQ ID NO: 145 - protein, 9 aa, Artificial Sequence
  peptide
WMYTFLISL

SEQ ID NO: 146 - protein, 9 aa, Artificial Sequence
  peptide
WMYTFLISV

SEQ ID NO: 147 - protein, 9 aa, Artificial Sequence
  peptide
WSDDGIWSI

SEQ ID NO: 148 - protein, 9 aa, Artificial Sequence
  peptide
WSDDGIWSL

SEQ ID NO: 149 - protein, 9 aa, Artificial Sequence
  peptide
WSDDGIWSV

SEQ ID NO: 150 - protein, 9 aa, Artificial Sequence
  peptide
WSDKQCWEI

SEQ ID NO: 151 - protein, 9 aa, Artificial Sequence
  peptide
WSDKQCWEL

SEQ ID NO: 152 - protein, 9 aa, Artificial Sequence
  peptide
WSDKQCWEV

SEQ ID NO: 153 - protein, 9 aa, Artificial Sequence
  peptide
WSDYKDFYI

SEQ ID NO: 154 - protein, 9 aa, Artificial Sequence
  peptide
WSDYKDFYL

SEQ ID NO: 155 - protein, 9 aa, Artificial Sequence
  peptide
WSDYKDFYV

SEQ ID NO: 156 - protein, 9 aa, Artificial Sequence
  peptide
WVQDMCVYY

SEQ ID NO: 157 - protein, 9 aa, Artificial Sequence
  peptide
YASDYKDFY

SEQ ID NO: 158 - protein, 9 aa, Artificial Sequence
  peptide
YIGCLYTFI

SEQ ID NO: 159 - protein, 9 aa, Artificial Sequence
  peptide
YIGCLYTFL

SEQ ID NO: 160 - protein, 9 aa, Artificial Sequence
  peptide
YIGCLYTFV

SEQ ID NO: 161 - protein, 9 aa, Artificial Sequence
  peptide
YIVKPLPPV

SEQ ID NO: 162 - protein, 9 aa, Artificial Sequence
  peptide
YLCSWKPGV

SEQ ID NO: 163 - protein, 9 aa, Artificial Sequence
  peptide
YLDDGIWSI

SEQ ID NO: 164 - protein, 9 aa, Artificial Sequence
  peptide
YLDDGIWSL

SEQ ID NO: 165 - protein, 9 aa, Artificial Sequence
  peptide
YLDDGIWSV

SEQ ID NO: 166 - protein, 9 aa, Artificial Sequence
  peptide
YLDHALQCV

SEQ ID NO: 167 - protein, 9 aa, Artificial Sequence
  peptide
YLDKQCWEV

SEQ ID NO: 168 - protein, 9 aa, Artificial Sequence
  peptide
YLFYWYEGI

SEQ ID NO: 169 - protein, 9 aa, Artificial Sequence
  peptide
YLFYWYEGL

SEQ ID NO: 170 - protein, 9 aa, Artificial Sequence
  peptide
YLFYWYEGV

SEQ ID NO: 171 - protein, 9 aa, Artificial Sequence
  peptide
YLGCLYTFI

SEQ ID NO: 172 - protein, 9 aa, Artificial Sequence
  peptide
YLGCLYTFL

SEQ ID NO: 173 - protein, 9 aa, Artificial Sequence
  peptide
YLGCLYTFV

SEQ ID NO: 174 - protein, 9 aa, Artificial Sequence
  peptide
YLISTTFGI

SEQ ID NO: 175 - protein, 9 aa, Artificial Sequence
  peptide
YLISTTFGL

SEQ ID NO: 176 - protein, 9 aa, Artificial Sequence
  peptide
YLISTTFGV

SEQ ID NO: 177 - protein, 9 aa, Artificial Sequence
  peptide
YLNETYTLK

SEQ ID NO: 178 - protein, 9 aa, Artificial Sequence
  peptide
YLPFGFILV

SEQ ID NO: 179 - protein, 9 aa, Artificial Sequence
  peptide
YLQDMCVYY

SEQ ID NO: 180 - protein, 9 aa, Artificial Sequence
  peptide
YLSDYKDFY

SEQ ID NO: 181 - protein, 9 aa, Artificial Sequence
  peptide
YLTNYNLFY

SEQ ID NO: 182 - protein, 9 aa, Artificial Sequence
  peptide
YLVKPLPPI

SEQ ID NO: 183 - protein, 9 aa, Artificial Sequence
  peptide
YLVKPLPPL

SEQ ID NO: 184 - protein, 9 aa, Artificial Sequence
  peptide
YLVKPLPPV

SEQ ID NO: 185 - protein, 9 aa, Artificial Sequence
  peptide
YLWKTIITK

SEQ ID NO: 186 - protein, 9 aa, Artificial Sequence
  peptide
YLWQPPLSI

SEQ ID NO: 187 - protein, 9 aa, Artificial Sequence
  peptide
YLWQPPLSL

SEQ ID NO: 188 - protein, 9 aa, Artificial Sequence
  peptide
YLWQPPLSV

SEQ ID NO: 189 - protein, 9 aa, Artificial Sequence
  peptide
YLYLLCSWK

SEQ ID NO: 190 - protein, 9 aa, Artificial Sequence
  peptide
YLYTFLISI

SEQ ID NO: 191 - protein, 9 aa, Artificial Sequence
  peptide
YLYTFLISL

SEQ ID NO: 192 - protein, 9 aa, Artificial Sequence
  peptide
YLYTFLIST

SEQ ID NO: 193 - protein, 9 aa, Artificial Sequence
  peptide
YLYTFLISV

SEQ ID NO: 194 - protein, 9 aa, Artificial Sequence
  peptide
YMCSWKPGI

SEQ ID NO: 195 - protein, 9 aa, Artificial Sequence
  peptide
YMCSWKPGV

SEQ ID NO: 196 - protein, 9 aa, Artificial Sequence
  peptide
YMDDGIWSI

SEQ ID NO: 197 - protein, 9 aa, Artificial Sequence
  peptide
YMDDGIWSL

SEQ ID NO: 198 - protein, 9 aa, Artificial Sequence
  peptide
YMDDGIWSV

SEQ ID NO: 199 - protein, 9 aa, Artificial Sequence
  peptide
YMDHALQCV

SEQ ID NO: 200 - protein, 9 aa, Artificial Sequence
  peptide
YMDKQCWEV

SEQ ID NO: 201 - protein, 9 aa, Artificial Sequence
  peptide
YMFYWYEGI

SEQ ID NO: 202 - protein, 9 aa, Artificial Sequence
  peptide
YMFYWYEGL

SEQ ID NO: 203 - protein, 9 aa, Artificial Sequence
  peptide
YMFYWYEGV

SEQ ID NO: 204 - protein, 9 aa, Artificial Sequence
  peptide
YMGCLYTFI

SEQ ID NO: 205 - protein, 9 aa, Artificial Sequence
  peptide
YMGCLYTFL

SEQ ID NO: 206 - protein, 9 aa, Artificial Sequence
  peptide
YMGCLYTFV

SEQ ID NO: 207 - protein, 9 aa, Artificial Sequence
  peptide
YMISTTFGI

SEQ ID NO: 208 - protein, 9 aa, Artificial Sequence
  peptide
YMISTTFGL

SEQ ID NO: 209 - protein, 9 aa, Artificial Sequence
  peptide
YMISTTFGV

SEQ ID NO: 210 - protein, 9 aa, Artificial Sequence
  peptide
YMLAIGCLY

SEQ ID NO: 211 - protein, 9 aa, Artificial Sequence
  peptide
YMNETYTLK

SEQ ID NO: 212 - protein, 9 aa, Artificial Sequence
  peptide
YMPFGFILV

SEQ ID NO: 213 - protein, 9 aa, Artificial Sequence
  peptide
YMQDMCVYY

SEQ ID NO: 214 - protein, 9 aa, Artificial Sequence
  peptide
YMSDYKDFY

SEQ ID NO: 215 - protein, 9 aa, Artificial Sequence
  peptide
YMTGLLLRK

SEQ ID NO: 216 - protein, 9 aa, Artificial Sequence
  peptide
YMTNYNLFY

SEQ ID NO: 217 - protein, 9 aa, Artificial Sequence
  peptide
YMVIFVTGV

SEQ ID NO: 218 - protein, 9 aa, Artificial Sequence
  peptide
YMVKPLPPI

SEQ ID NO: 219 - protein, 9 aa, Artificial Sequence
  peptide
YMVKPLPPL

SEQ ID NO: 220 - protein, 9 aa, Artificial Sequence
  peptide
YMVKPLPPV

SEQ ID NO: 221 - protein, 9 aa, Artificial Sequence
  peptide
YMWKTIITK

SEQ ID NO: 222 - protein, 9 aa, Artificial Sequence
  peptide
YMWQPPLSI

SEQ ID NO: 223 - protein, 9 aa, Artificial Sequence
  peptide
YMWQPPLSL

SEQ ID NO: 224 - protein, 9 aa, Artificial Sequence
  peptide
YMWQPPLSV

SEQ ID NO: 225 - protein, 9 aa, Artificial Sequence
  peptide
YMYLLCSWK

SEQ ID NO: 226 - protein, 9 aa, Artificial Sequence
  peptide
YMYTFLISI

SEQ ID NO: 227 - protein, 9 aa, Artificial Sequence
  peptide
YMYTFLISL

SEQ ID NO: 228 - protein, 9 aa, Artificial Sequence
  peptide
YMYTFLIST

SEQ ID NO: 229 - protein, 9 aa, Artificial Sequence
  peptide
YMYTFLISV

SEQ ID NO: 230 - protein, 9 aa, Artificial Sequence
  peptide
YQWQPPLSV

SEQ ID NO: 231 - protein, 9 aa, Artificial Sequence
  peptide
YSDDGIWSI

SEQ ID NO: 232 - protein, 9 aa, Artificial Sequence
  peptide
YSDDGIWSL

SEQ ID NO: 233 - protein, 9 aa, Artificial Sequence
  peptide
YSDDGIWSV

SEQ ID NO: 234 - protein, 9 aa, Artificial Sequence
  peptide
YSDKQCWEI

SEQ ID NO: 235 - protein, 9 aa, Artificial Sequence
  peptide
YSDKQCWEL

SEQ ID NO: 236 - protein, 9 aa, Artificial Sequence
  peptide
YSDKQCWEV

SEQ ID NO: 237 - protein, 9 aa, Artificial Sequence
  peptide
YSDYKDFYI

SEQ ID NO: 238 - protein, 9 aa, Artificial Sequence
  peptide
YSDYKDFYL

SEQ ID NO: 239 - protein, 9 aa, Artificial Sequence
  peptide
YSDYKDFYV

SEQ ID NO: 240 - protein, 9 aa, Artificial Sequence
  peptide
YSSWAETTY

SEQ ID NO: 241 - protein, 9 aa, Artificial Sequence
  peptide
YTWKTIITK

SEQ ID NO: 242 - protein, 9 aa, Artificial Sequence
  peptide
YVQDMCVYY

SEQ ID NO: 243 - protein, 9 aa, Artificial Sequence
  peptide
AIGCLYTFL

SEQ ID NO: 244 - protein, 9 aa, Artificial Sequence
  peptide
ASDYKDFYI

SEQ ID NO: 245 - protein, 9 aa, Artificial Sequence
  peptide
CLYTFLIST

SEQ ID NO: 246 - protein, 9 aa, Artificial Sequence
  peptide
CSDDGIWSE

SEQ ID NO: 247 - protein, 9 aa, Artificial Sequence
  peptide
EASDYKDFY

SEQ ID NO: 248 - protein, 9 aa, Artificial Sequence
  peptide
ETWKTIITK

SEQ ID NO: 249 - protein, 9 aa, Artificial Sequence
  peptide
FLISTTFGC

SEQ ID NO: 250 - protein, 9 aa, Artificial Sequence
  peptide
FVTGLLLRK

SEQ ID NO: 251 - protein, 9 aa, Artificial Sequence
  peptide
GLDHALQCV

SEQ ID NO: 252 - protein, 9 aa, Artificial Sequence
  peptide
ILVIFVTGL

SEQ ID NO: 253 - protein, 9 aa, Artificial Sequence
  peptide
KVQDMCVYY

SEQ ID NO: 254 - protein, 9 aa, Artificial Sequence
  peptide
LDTNYNLFY

SEQ ID NO: 255 - protein, 9 aa, Artificial Sequence
  peptide
LLCSWKPGI

SEQ ID NO: 256 - protein, 9 aa, Artificial Sequence
  peptide
LQWQPPLSL

SEQ ID NO: 257 - protein, 9 aa, Artificial Sequence
  peptide
NIVKPLPPV

SEQ ID NO: 258 - protein, 9 aa, Artificial Sequence
  peptide
NLFYWYEGL

SEQ ID NO: 259 - protein, 9 aa, Artificial Sequence
  peptide
QSSWAETTY

SEQ ID NO: 260 - protein, 9 aa, Artificial Sequence
  peptide
RNIGSETWK

SEQ ID NO: 261 - protein, 9 aa, Artificial Sequence
  peptide
VCLAIGCLY

SEQ ID NO: 262 - protein, 9 aa, Artificial Sequence
  peptide
VENETYTLK

SEQ ID NO: 263 - protein, 9 aa, Artificial Sequence
  peptide
WLPFGFILI

SEQ ID NO: 264 - protein, 9 aa, Artificial Sequence
  peptide
WQYLLCSWK

SEQ ID NO: 265 - protein, 9 aa, Artificial Sequence
  peptide
WSDKQCWEG

SEQ ID NO: 266 - protein, 20 aa, Artificial Sequence
  peptide
MAKTIAYDEEARRGLERGLN

SEQ ID NO: 267 - protein, 9 aa, Artificial Sequence
  peptide
YLPFGFILV

SEQ ID NO: 268 - protein, 9 aa, Artificial Sequence
  peptide
VLPFGFILV

SEQ ID NO: 269 - protein, 9 aa, Artificial Sequence
  peptide
NLPFGFILV

SEQ ID NO: 270 - protein, 9 aa, Artificial Sequence
  peptide
RLPFGFILV

SEQ ID NO: 271 - protein, 9 aa, Artificial Sequence
  peptide
SLPFGFILV

SEQ ID NO: 272 - protein, 9 aa, Artificial Sequence
  peptide
TLPFGFILV

SEQ ID NO: 273 - protein, 9 aa, Artificial Sequence
  peptide
CLPFGFILV

SEQ ID NO: 274 - protein, 9 aa, Artificial Sequence
  peptide
LLPFGFILV

SEQ ID NO: 275 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 276 - protein, 10 aa, Artificial Sequence
  peptide
WLPFGFILIL

SEQ ID NO: 277 - protein, 9 aa, Artificial Sequence
  peptide
VLLDTNYNL

SEQ ID NO: 278 - protein, 9 aa, Artificial Sequence
  peptide
GLDHALQCV

SEQ ID NO: 279 - protein, 10 aa, Artificial Sequence
  peptide
FLPFGFILPV

SEQ ID NO: 280 - protein, 13 aa, Artificial Sequence
  peptide
TPPAYRPPNAPIL

SEQ ID NO: 281 - protein, 15 aa, Artificial Sequence
  peptide
KSVWSKLQSIGIRQH

SEQ ID NO: 282 - protein, 9 aa, Artificial Sequence
  peptide
ALPFGFILV

SEQ ID NO: 283 - protein, 9 aa, Artificial Sequence
  peptide
WLPFGFILV

SEQ ID NO: 284 - protein, 9 aa, Artificial Sequence
  peptide
ELPFGFILV

(19) United States
(12) Patent Application Publication
(10) Pub. No.: US 2021/0106652 A1
(43) Pub. Date: Apr. 15, 2021

(54) Immunogenic Compounds For Treatment Of Fibrosis, Autoimmune Diseases And Inflammation

(71) Applicant: ENTEROME S.A., Paris (FR)

(72) Inventors: Laurent CHENE, Neuville Aux Bois (FR); Christophe BONNY, Paris (FR); Francesco STROZZI, Paris (FR)

(21) Appl. No.: 17/043,197

(22) PCT Filed: Apr. 11, 2019
(86) PCT No.: PCT/EP2019/059319

§ 371 Date: Sep. 29, 2020

(30) Foreign Application Priority Data
Apr. 11, 2018 (EP) 18305443.6

(51) Int. Cl. A61K 38/17; A61P 29/00; A61K 9/51

(52) U.S. Cl. CPC A61K 38/1793; A61K 9/51; A61P 29/00

(57) ABSTRACT

The present invention relates to antigen-based immunotherapy targeting interleukin 13 receptor alpha 2 (I LI 3RA2) for prevention and/or treatment of fibrosis, an autoimmune disease and/or an inflammatory disease. In particular, the present invention provides the use of a (poly)peptide comprising an epitope of IL13RA2 or a sequence variant thereof for prevention and/or treatment of fibrosis, an autoimmune disease and/or an inflammatory disease. Moreover, the present invention also provides an immunogenic compound, a nanoparticle and a pharmaceutical composition comprising such a (poly)peptide and a nucleic acid encoding such a (poly)peptide for use in prevention and/or treatment of fibrosis, an autoimmune disease and/or an inflammatory disease.

The present invention relates to the field of immunotherapy of fibrosis, autoimmune diseases and an inflammatory disease. In particular, the present invention relates to antigen-based immunotherapy targeting interleukin-13 receptor α2 (IL13RA2) for prevention and treatment of fibrosis, autoimmune diseases and an inflammatory disease.

Interleukin 13 (also referred to as “IL-13” or “IL13”) belongs to the group of T helper type 2 (Th2) cytokines, i.e. to the subset of cytokines produced by Th2 cells, which includes, for example, IL-4, IL-5, IL-6, IL-9, IL-13, and IL-17E (IL-25). Th2 cytokines have effects on many cell types in the body. For example, Th2 cells are known to stimulate and recruit specialized subsets of immune cells (e.g., eosinophils and basophils) to the site of infection or in response to allergens and to mediate host defense against parasites. In addition, Th2 cells are known to induce mucus production, goblet cell metaplasia, and airway hyper-responsiveness. Th2 cells also control the regulation of B cell class-switching to IgE. In summary, Th2 cells influence the production of antibodies and allergic responses and, therefore, overactivation of Th2 cells appears to be responsible for the exacerbation of allergies, autoimmune reactions such as chronic graft-versus-host disease, progressive systemic sclerosis, and systemic lupus erythematosus. Moreover, Th2 cells are also known to be responsible for the development of asthma and other allergic inflammatory diseases.

Functionally, the effects of IL-13 on immune cells are similar to those of the closely related Th2 cytokine IL-4. IL-4 and IL-13 share 20-25% sequence identity in their amino acid sequences with higher % identity in the first and in the last alpha-helical regions, which are known to be key for IL-4 activity. Accordingly, the signaling of IL-13 begins through a shared multi-subunit receptor with IL-4 and the “IL-4/IL-13 axis” was implicated in a variety of diseases including asthma, atopic dermatitis, allergic rhinitis, COPD, cancer, IBD, autoimmune diseases and fibrosis (for review see May R D, Fung M. Strategies targeting the IL-4/IL-13 axes in disease. Cytokine. 2015 September; 75(1):89-116). The shared multi-subunit IL-4/IL-13 receptor, which is known to mediate those effects, is a heterodimer receptor complex comprising alpha IL-4 receptor (IL-4Rα) and alpha Interleukin-13 receptor (IL13R1). This receptor complex allows for the downstream activation of signal transducer and activator of transcription 6 (STAT6). Indeed, most biological effects of IL-13 and IL-4 are linked to STAT6 activation.

In addition, IL-13 binds also to another receptor, interleukin-13 receptor α2 (IL13RA2). IL13RA2 contains only a 7-amino-acid cytoplasmatic domain lacking a conserved box 1 region that is known to play a critical role in signal transduction. In view of this lack of a significant cytoplasmatic tail, IL13RA2 is generally believed to act only as “decoy” for IL-13, i.e. as negative regulator for IL-13-induced responses. Accordingly, IL13RA2 is often termed “decoy receptor” and suggested as therapy in diseases involving overactivation of IL-13. This was supported by findings that administration of the soluble form of IL13RA2 suppresses IL-13-mediated airway inflammation, airway hyperresponsiveness, and mucus hypersecretion in allergen-challenged mice. Therefore, it is widely believed that the above-described diseases involving IL-13 are mediated via the IL4Rα/IL13R1 receptor, whereas IL13RA2 counteracts IL-13-induced responses and was, thus, suggested as therapy (see, for example, Wynn T A. Cellular and molecular mechanisms of fibrosis. J Pathol. 2008 January; 214(2):199-210).

Recently, however, a distinct picture of the functional roles of IL13RA2 emerges. Many of the above-described experimental data were obtained in mice, and it was recently found that both, a soluble form of IL13RA2 (often termed “sIL13RA2”) and a membrane-bound form of IL13RA2 (often termed “memIL13RA2”), which are encoded by distinct transcripts generated by alternative splicing exist in vivo in mice (Tabata Y, Chen W, Warrier M R, Gibson A M, Daines M O, Hershey G K. Allergy-driven alternative splicing of IL-13 receptor alpha2 yields distinct membrane and soluble forms. J Immunol. 2006 Dec. 1; 177(11):7905-12). In humans, in contrast, IL13RA2 exists only in its membrane-bound form (Chen W, Sivaprasad U, Tabata Y, Gibson A M, Stier M T, Finkelman F D, Hershey G K. IL-13R alpha 2 membrane and soluble isoforms differ in humans and mice. J Immunol. 2009 Dec. 15; 183(12):7870-6; O'Toole M, Legault H, Ramsey R, Wynn T A, Kasaian M T. A novel and sensitive ELISA reveals that the soluble form of IL-13R-alpha2 is not expressed in plasma of healthy or asthmatic subjects. Clin Exp Allergy. 2008 April; 38(4):594-601).

Moreover, recent studies demonstrated that IL13RA2 can indeed mediate IL-13 signaling. In a landmark publication, Fichtner-Feigl et al. reported that prevention of IL13RA2 expression, IL13RA2 gene silencing by siRNA and blockade of IL13RA2 downstream signaling resulted in a marked downregulation of transforming growth factor β1 (TGF-β1) production and bleomycin-induced lung fibrosis (Fichtner-Feigl 5, Strober W, Kawakami K, Puri R K, Kitani A. IL-13 signaling through the IL-13alpha2 receptor is involved in induction of TGF-beta1 production and fibrosis. Nat Med. 2006 January; 12(1):99-106). The authors show that IL13RA2 stimulates an activator protein (AP-1) variant containing c-jun and fra-2, which initiates transcription at the TGF-β1 promoter, thereby inducing TGF-β1 production. Another interesting study suggested that IL13RA2 forms a heterodimeric signaling complex with IL-13 and the chitinase 3-like 1 protein family member (Chi3I1), which activates MAPK, B/AKT and Wnt/B-catenin signaling pathways, thereby regulating oxidant injury, apoptosis, pyroptosis, inflammasome activation, antibacterial responses, melanoma metastasis and TGF-β1 production, (He C H, Lee C G, Dela Cruz C S, Lee C M, Zhou Y, Ahangari F, Ma B, Herzog E L, Rosenberg S A, Li Y, Nour A M, Parikh C R, Schmidt I, Modis Y, Cantley L, Elias J A. Chitinase 3-like 1 regulates cellular and tissue responses via IL-13 receptor α2. Cell Rep. 2013 Aug. 29; 4(4):830-41). Moreover, IL13RA2 was also shown to influence IL13RA1 signaling pathways in complex ways (Strober W, Kitani A, Fichtner-Feigl S, Fuss I J. The signaling function of the IL-13Ralpha2 receptor in the development of gastrointestinal fibrosis and cancer surveillance. Curr Mol Med. 2009 August; 9(6):740-50).

In addition, it was reported that IL-13 signaling via IL13RA2 initiates a complex fibrotic program in the colon including TGF-β1 activation, IGF-1 and Egr-1 expression, myofibroblast apoptosis, and myofibroblast production of collagen (Fichtner-Feigl S, Young C A, Kitani A, Geissler E K, Schlitt H J, Strober W. IL-13 signaling via IL-13R alpha2 induces major downstream fibrogenic factors mediating fibrosis in chronic TNBS colitis. Gastroenterology. 2008 December; 135(6):2003-13).

Moreover, IL13RA2 was found to be expressed in hepatic stellate cells (HSC) in sinusoidal lesions of the liver in patients suffering from non-alcoholic steatohepatitis (NASH)—but not in normal and fatty liver tissues (Shimamura T, Fujisawa T, Husain S R, Kioi M, Nakajima A, Puri R K. Novel role of IL-13 in fibrosis induced by nonalcoholic steatohepatitis and its amelioration by IL-13R-directed cytotoxin in a rat model. J Immunol. 2008 Oct. 1; 181(7):4656-65). The authors of this study report that in a rat model of NASH, treatment with IL13-PE38 (a cytotoxin targeting IL13RA2, Kioi M, Seetharam S, Puri R K. Targeting IL-13Ralpha2-positive cancer with a novel recombinant immunotoxin composed of a single-chain antibody and mutated Pseudomonas exotoxin. Mol Cancer Ther. 2008 June; 7(6):1579-87) mediated remarkable antifibrotic effects (Shimamura et al., 2008).

Furthermore, IL13RA2 was also reported to be involved in the etiology of proliferative vitreoretinopathy (Abu El-Asrar A M, Missotten L, Geboes K. Expression of myofibroblast activation molecules in proliferative vitreoretinopathy epiretinal membranes. Acta Ophthalmol. 2011 March; 89(2):e115-21), allograft fibrosis (Brunner S M, Schiechl G, Kesselring R, Martin M, Balam S, Schlitt H J, Geissler E K, Fichtner-Feigl S. IL-13 signaling via IL-13Rα2 triggers TGF-β1-dependent allograft fibrosis. Transplant Res. 2013 Oct. 22; 2(1):16) and allergic asthma (Chen W, Sivaprasad U, Gibson A M, Ericksen M B, Cunningham C M, Bass S A, κinker K G, Finkelman F D, Wills-Karp M, Khurana Hershey G K. IL-13 receptor α2 contributes to development of experimental allergic asthma. J Allergy Clin Immunol. 2013 October; 132(4):951-8.e1-6).

In summary, IL13RA2 recently emerged as novel therapeutic target in fibrosis, autoimmune diseases and disorders and inflammatory diseases. In view thereof, it is the object of the present invention to provide a novel approach in targeting IL13RA2 in order to prevent and/or treat fibrosis, autoimmune diseases/disorders and/or inflammatory diseases. In particular, it is the object of the present invention to provide a novel approach in targeting IL13RA2 overexpressing cells and/or cells, which express IL13RA2 solely under pathological conditions, such as in fibrosis, autoimmune diseases/disorders and/or inflammatory diseases. As a specific example, it is an object of the invention to provide a novel approach to eliminate hepatic stellate cells expressing IL13RA2 in NASH patients in order to treat (fibrosis associated with) NASH.

This object is achieved by means of the subject-matter set out below, in particular in the items provided by the present invention and in the appended claims.

### ITEMS OF THE INVENTION

The present invention provides in particular the following items:
1. A (poly)peptide comprising an epitope of IL13RA2 or a sequence variant thereof having at least 70% sequence identity for use in prevention and/or treatment of fibrosis, an autoimmune disease and/or an inflammatory disease.
2. The (poly)peptide for use according to item 1, wherein the epitope of IL13RA2 or the sequence variant thereof has a length of 8-12 amino acids, more preferably of 8-10 amino acids and most preferably of 9 or 10 amino acids.
3. The (poly)peptide for use according to item 1 or 2, wherein the epitope of IL13RA2 is an epitope of human IL13RA2.
4. The (poly)peptide for use according to item 3, wherein the epitope of IL13RA2 has an amino acid sequence as set forth in any one of SEQ ID NOs 243-265 and 276-278.
5. The (poly)peptide for use according to any one of items 1-4 comprising a sequence variant of the IL13RA2 epitope, wherein the core sequence of the sequence variant is identical with the core sequence of the IL13RA2 epitope, with the core sequence consisting of all amino acids except the three most N-terminal and the three most C-terminal amino acids.
6. The (poly)peptide for use according to any one of items 1-5 comprising a sequence variant of the IL13RA2 epitope, wherein the sequence variant is a microbiota sequence variant.
7. The (poly)peptide for use according to item 6, wherein the microbiota sequence variant is a bacterial peptide.
8. The (poly)peptide for use according to any one of items 1-7 comprising an amino acid sequence as set forth in any one of SEQ ID NOs 1-242, 267-274, and 279, preferably as set forth in any one of SEQ ID NOs 31, 64, 178, 192, 212, 267 and 279.
9. The (poly)peptide for use according to item 8, wherein the (poly)peptide comprises an amino acid sequence as set forth in any one of SEQ ID NOs 31, 192 or 279, preferably wherein the (poly)peptide comprises an amino acid sequence as set forth in SEQ ID NO: 31 or 192.
10. The (poly)peptide for use according to item 8, wherein the (poly)peptide comprises an amino acid sequence as set forth in SEQ ID NO: 31.
11. An immunogenic compound comprising the (poly)peptide as defined in any one of items 1-10 for use in prevention and/or treatment of fibrosis, an autoimmune disease and/or an inflammatory disease.
12. The immunogenic compound for use according to item 11, wherein the (poly)peptide is linked to a carrier protein.
13. The immunogenic compound for use according to item 11 or 12 comprising or consisting of an (poly)peptide of formula (I):

PepNt-CORE-PepCt (I),
wherein:
   “PepNt” consists of a polypeptide having an amino acid length varying from 0 to 500 amino acid residues and located at the N-terminal end of the polypeptide of formula (I);
   CORE consists of a (poly)peptide as defined in any one of items 1-10; and
   “PepCt” consists of a polypeptide having an amino acid length varying from 0 to 500 amino acid residues and located at the C-terminal end of the polypeptide of formula (I).
14. A nanoparticle loaded with
   at least one (poly)peptide as defined in any one of items 1-10, or
   at least one immunogenic compound as defined in any one of items 11-13;
   and, optionally, with an adjuvant
   for use in prevention and/or treatment of fibrosis, an autoimmune disease and/or an inflammatory disease.
15. A cell loaded with the (poly)peptide as defined in any one of items 1-10 or the immunogenic compound as defined in any one of items 11-13 for use in prevention and/or treatment of fibrosis, an autoimmune disease and/or an inflammatory disease.
16. The cell for use according to item 15, wherein said cell is an antigen presenting cell, preferably a dendritic cell.
17. A nucleic acid encoding a (poly)peptide as defined in any one of items 1-10 for use in prevention and/or treatment of fibrosis, an autoimmune disease and/or an inflammatory disease.
18. The nucleic acid for use according to item 17, wherein the nucleic acid is a DNA molecule or an RNA molecule; preferably selected from genomic DNA; cDNA; siRNA; rRNA; mRNA; antisense DNA; antisense RNA; ribozyme; complimentary RNA and/or DNA sequences; RNA and/or DNA sequences with or without expression elements, regulatory elements, and/or promoters; a vector; and combinations thereof.
19. A host cell comprising the nucleic acid as defined in item 17 or 18 for use in prevention and/or treatment of fibrosis, an autoimmune disease and/or an inflammatory disease, wherein the nucleic acid is preferably a vector.
20. The host cell for use according to item 19, wherein the host cell is a bacterial cell, preferably a gut bacterial cell.
21. A pharmaceutical composition comprising
   the (poly)peptide as defined in any one of items 1-10;
   the immunogenic compound as defined in any one of items 11-13;
   the nanoparticle as defined in item 14;
   the cell as defined in item 15 or 16;
   the nucleic acid as defined in item 17 or 18; or
   the host cell as defined in item 19 or 20,
   and, optionally, one or more pharmaceutically acceptable excipients
   for use in prevention and/or treatment of fibrosis, an autoimmune disease and/or an inflammatory disease.
22. The pharmaceutical composition for use according to item 21 further comprising one or more immunostimulatory agents.
23. The pharmaceutical composition for use according to item 22, wherein the said immunostimulatory agent is selected in a group consisting of immuno-adjuvants and antigen-presenting cells.
24. The pharmaceutical composition for use according to item 23, wherein the antigen-presenting cells are dendritic cells.
25. The pharmaceutical composition for use according to any one of items 21-24, wherein the composition comprises
   (i) at least two distinct (poly)peptides as defined in any one of items 1-10;
   (ii) at least two distinct immunogenic compounds as defined in any one of items 11-13, wherein each of the at least two immunogenic compounds comprises a distinct (poly)peptide as defined in any one of items 1-10;
   (iii) at least two distinct nanoparticles as defined in item 14, wherein each of the at least two nanoparticles is loaded with a distinct (poly)peptide as defined in any one of items 1-10; or
   (iv) at least two distinct nucleic acids as defined in item 17 or 18, wherein each of the at least two nucleic acids encodes a distinct (poly)peptide as defined in any one of items 1-10.
26. The pharmaceutical composition for use according to item 25, wherein the composition comprises
   (i) a (poly)peptide comprising an amino acid sequence as set forth in SEQ ID NO:
31 or 279 and a (poly)peptide comprising an amino acid sequence as set forth in SEQ ID NO: 192;
   (ii) an immunogenic compound comprising a (poly)peptide comprising an amino acid sequence as set forth in SEQ ID NO: 31 or 279 and an immunogenic compound comprising a (poly)peptide comprising an amino acid sequence as set forth in SEQ ID NO: 192;
   (iii) a nanoparticle loaded with a (poly)peptide comprising an amino acid sequence as set forth in SEQ ID NO: 31 or 279 and a nanoparticle loaded with a (poly)peptide comprising an amino acid sequence as set forth in SEQ ID NO: 192; or
   (iv) a nucleic acid comprising a polynucleotide encoding an (poly)peptide having an amino acid sequence as set forth in SEQ ID NO: 31 or 279 and a nucleic acid comprising a polynucleotide encoding an (poly)peptide having an amino acid sequence as set forth in SEQ ID NO: 192.
27. A combination of
   (i) a (poly)peptide as defined in any one of items 1-10 comprising an amino acid sequence as set forth in SEQ ID NO: 31 or 279, and
   (ii) a (poly)peptide as defined in any one of items 1-10 comprising an amino acid sequence as set forth in SEQ ID NO: 192
   for use in prevention and/or treatment of fibrosis, an autoimmune disease and/or an inflammatory disease.
28. A combination of
   (i) an immunogenic compound as defined in any one of items 11-13 comprising a (poly)peptide comprising an amino acid sequence as set forth in SEQ ID NO: 31 or 279, and
   (ii) an immunogenic compound as defined in any one of items 11-13 comprising a (poly)peptide comprising an amino acid sequence as set forth in SEQ ID NO: 192
   for use in prevention and/or treatment of fibrosis, an autoimmune disease and/or an inflammatory disease.
29. A combination of
   (i) a nanoparticle according to item 14 comprising a (poly)peptide comprising an amino acid sequence as set forth in SEQ ID NO: 31 or 279, and
   (ii) a nanoparticle according to item 14 comprising a (poly)peptide comprising an amino acid sequence as set forth in SEQ ID NO: 192
   for use in prevention and/or treatment of fibrosis, an autoimmune disease and/or an inflammatory disease.
30. A combination of
   (i) a nucleic acid according to item 17 or 18 encoding a (poly)peptide comprising an amino acid sequence as set forth in SEQ ID NO: 31 or 279, and
   (ii) a nucleic acid according to item 17 or 18 encoding a (poly)peptide comprising an amino acid sequence as set forth in SEQ ID NO: 192
   for use in prevention and/or treatment of fibrosis, an autoimmune disease and/or an inflammatory disease.
31. The combination for use according to any one of items 27-30, wherein components (i) and (ii) are comprised in distinct compositions.
32. The combination for use according to any one of items 27-30, wherein components (i) and (ii) are comprised in the same composition.
33. The combination for use according to any one of items 27-31, wherein components (i) and (ii) are administered via distinct routes of administration.
34. The combination for use according to any one of items 27-32, wherein components (i) and (ii) are administered via the same route of administration.
35. The combination for use according to any one of items 27-31, 33 and 34 wherein components (i) and (ii) are administered consecutively.
36. The combination for use according to any one of items 27-34 wherein components (i) and (ii) are administered at about the same time.
37. A kit comprising
   the (poly)peptide as defined in any one of items 1-10;
   the immunogenic compound as defined in any one of items 11-13;
   the nanoparticle as defined in item 14;
   the cell as defined in item 15 or 16;
   the nucleic acid as defined in item 17 or 18;
   the host cell as defined in item 19 or 20; and/or
   the pharmaceutical composition as defined in any one of items 21-26
   for use in prevention and/or treatment of fibrosis, an autoimmune disease and/or an inflammatory disease.
38. The kit for use according to item 37 further comprising a package insert or instruction leaflet with directions to prevent and/or to treat fibrosis, an autoimmune disease and/or an inflammatory disease by using the (poly)peptide as defined in any one of items 1-10; the immunogenic compound as defined in any one of items 11-13; the nanoparticle as defined in item 14; the cell as defined in item 15 or 16; the nucleic acid as defined in item 17 or 18; the host cell as defined in item 19 or 20; and/or the pharmaceutical composition as defined in any one of items 21-26.
39. The kit for use according to item 37 or 38, wherein the kit comprises
   (i) a (poly)peptide as defined in any one of items 1-10 comprising an amino acid sequence as set forth in SEQ ID NO: 31 or 279, and
   (ii) a (poly)peptide as defined in any one of items 1-10 comprising an amino acid sequence as set forth in SEQ ID NO: 192.
40. The kit for use according to item 37 or 38, wherein the kit comprises
   (i) an immunogenic compound as defined in any one of items 11-13 comprising a (poly)peptide comprising an amino acid sequence as set forth in SEQ ID NO: 31 or 279, and
   (ii) an immunogenic compound as defined in any one of items 11-13 comprising a (poly)peptide comprising an amino acid sequence as set forth in SEQ ID NO: 192.
41. The kit for use according to item 37 or 38, wherein the kit comprises
   (i) a nanoparticle according to item 14 comprising a (poly)peptide comprising an amino acid sequence as set forth in SEQ ID NO: 31 or 279, and
   (ii) a nanoparticle according to item 14 comprising a (poly)peptide comprising an amino acid sequence as set forth in SEQ ID NO: 192.
42. The kit for use according to item 37 or 38, wherein the kit comprises
   (i) a nucleic acid according to item 17 or 18 encoding a (poly)peptide comprising an amino acid sequence as set forth in SEQ ID NO: 31 or 279, and
   (ii) a nucleic acid according to item 17 or 18 encoding a (poly)peptide comprising an amino acid sequence as set forth in SEQ ID NO: 192.
43. The (poly)peptide for use according to any one of items 1-10, the immunogenic compound for use according to any one of items 11-13, the nanoparticle for use according to item 14, the cell for use according to item 15 or 16, the nucleic acid for use according to item 17 or 18, the host cell for use according to item 19 or 20, the pharmaceutical composition according to any one of items 21-26, the combination for use according to any one of items 27-36, or the kit for use according to any one of items 37-42,
   wherein the disease/disorder to be treated and/or prevented is fibrosis.
44. The (poly)peptide, the immunogenic compound, the nanoparticle, the, the nucleic acid, the host cell, the pharmaceutical composition, the combination, or the kit for use according to item 43, wherein the fibrosis is selected from the group consisting of: a fibrosis of the lung, a fibrosis of the liver, a gastrointestinal fibrosis, a fibrosis of the heart, a fibrosis of the brain, a fibrosis of the skin, a fibrosis of a joint, such as shoulder and/or knee, a fibrosis of hands or feet, a fibrosis of the eye, a fibrosis of bone marrow and an allograft fibrosis.
45. The (poly)peptide, the immunogenic compound, the nanoparticle, the, the nucleic acid, the host cell, the pharmaceutical composition, the combination, or the kit for use according to item 43 or 44, wherein the fibrosis is selected from the group consisting of nonalcoholic steatohepatitis (NASH), cirrhosis, biliary atresia, schistosomiasis-induced hepatic fibrosis, systemic sclerosis, idiopathic pulmonary fibrosis (IPF), cystic fibrosis, radiation-induced lung injury, proliferative vitreoretinopathy, fibrosis in chronic TNBS colitis, fibrosis in Crohn's disease, allograft fibrosis in organ transplantation, atrial fibrosis, endomyocardial fibrosis, old myocardial fibrosis, glial scar, arthrofibrosis, mediastinal fibrosis, myelofibrosis, nephrogenic systemic fibrosis, progressive massive fibrosis, and retroperitoneal fibrosis.
46. The (poly)peptide, the immunogenic compound, the nanoparticle, the, the nucleic acid, the host cell, the pharmaceutical composition, the combination, or the kit for use according to any one of items 43-45, wherein the fibrosis is selected the group consisting of from nonalcoholic steatohepatitis (NASH), idiopathic pulmonary fibrosis (IPF), proliferative vitreoretinopathy, fibrosis in chronic TNBS colitis, and allograft fibrosis in organ transplantation.
47. The (poly)peptide, the immunogenic compound, the nanoparticle, the, the nucleic acid, the host cell, the pharmaceutical composition, the combination, or the kit for use according to any one of items 43-46, wherein the disease/disorder to be treated and/or prevented is nonalcoholic steatohepatitis (NASH).
48. The (poly)peptide for use according to any one of items 1-10, the immunogenic compound for use according to any one of items 11-13, the nanoparticle for use according to item 14, the cell for use according to item 15 or 16, the nucleic acid for use according to item 17 or 18, the host cell for use according to item 19 or 20, the pharmaceutical composition according to any one of items 21-26, the combination for use according to any one of items 27-36, or the kit for use according to any one of items 37-42,
   wherein the disease/disorder to be treated and/or prevented is an autoimmune disease/disorder.
49. The (poly)peptide for use according to any one of items 1-10, the immunogenic compound for use according to any one of items 11-13, the nanoparticle for use according to item 14, the cell for use according to item 15 or 16, the nucleic acid for use according to item 17 or 18, the host cell for use according to item 19 or 20, the pharmaceutical composition according to any one of items 21-26, the combination for use according to any one of items 27-36, or the kit for use according to any one of items 37-42,
   wherein the disease/disorder to be treated and/or prevented is an inflammatory disease.
50. The (poly)peptide, the immunogenic compound, the nanoparticle, the, the nucleic acid, the host cell, the pharmaceutical composition, the combination, or the kit for use according to item 48 or 49, wherein the disease/disorder to be treated and/or prevented is allergic inflammation.
51. The (poly)peptide, the immunogenic compound, the nanoparticle, the, the nucleic acid, the host cell, the pharmaceutical composition, the combination, or the kit for use according to any one of items 48-50, wherein the disease/disorder to be treated and/or prevented is asthma, preferably allergic asthma.
52. A method for preventing and/or treating fibrosis, an autoimmune disease and/or an inflammatory disease in a subject comprising administering to the subject
   the (poly)peptide as defined in any one of items 1-10;
   the immunogenic compound as defined in any one of items 11-13;
   the nanoparticle as defined in item 14;
   the cell as defined in item 15 or 16;
   the nucleic acid as defined in item 17 or 18;
   the host cell as defined in item 19 or 20;
   the pharmaceutical composition as defined in any one of items 21-26;
   the combination as defined in any one of items 27-36; and/or
   the kit as defined in any one of items 37-42.
53. The method according to item 52, wherein the disease/disorder to be treated and/or prevented is fibrosis.
54. The method according to item 53, wherein the fibrosis is selected from the group consisting of: a fibrosis of the lung, a fibrosis of the liver, a gastrointestinal fibrosis, a fibrosis of the heart, a fibrosis of the brain, a fibrosis of the skin, a fibrosis of a joint, such as shoulder and/or knee, a fibrosis of hands or feet, a fibrosis of the eye, a fibrosis of bone marrow and an allograft fibrosis.
55. The method according to item 53 or 54, wherein the fibrosis is selected from the group consisting of nonalcoholic steatohepatitis (NASH), cirrhosis, biliary atresia, schistosomiasis-induced hepatic fibrosis, systemic sclerosis, idiopathic pulmonary fibrosis (IPF), cystic fibrosis, radiation-induced lung injury, proliferative vitreoretinopathy, fibrosis in chronic TNBS colitis, fibrosis in Crohn's disease, allograft fibrosis in organ transplantation, atrial fibrosis, endomyocardial fibrosis, old myocardial fibrosis, glial scar, arthrofibrosis, mediastinal fibrosis, myelofibrosis, nephrogenic systemic fibrosis, progressive massive fibrosis, and retroperitoneal fibrosis.
56. The method according to any one of items 53-55, wherein the fibrosis is selected the group consisting of from nonalcoholic steatohepatitis (NASH), idiopathic pulmonary fibrosis (IPF), proliferative vitreoretinopathy, fibrosis in chronic TNBS colitis, and allograft fibrosis in organ transplantation.
57. The method according to any one of items 53-56, wherein the disease/disorder to be treated and/or prevented is nonalcoholic steatohepatitis (NASH).
58. The method according to item 52, wherein the disease/disorder to be treated and/or prevented is an autoimmune disease/disorder.
59. The method according to item 52, wherein the disease/disorder to be treated and/or prevented is an inflammatory disease.
60. The method according to item 58 or 59, wherein the disease/disorder to be treated and/or prevented is allergic inflammation.
61. The method according to any one of items 58-60, wherein the disease/disorder to be treated and/or prevented is asthma, preferably allergic asthma.
62. A method for eliciting an immune response against cells (over)expressing IL13RA2 in a subject comprising administering to the subject
   the (poly)peptide as defined in any one of items 1-10;
   the immunogenic compound as defined in any one of items 11-13;
   the nanoparticle as defined in item 14;
   the cell as defined in item 15 or 16;
   the nucleic acid as defined in item 17 or 18;
   the host cell as defined in item 19 or 20;
   the pharmaceutical composition as defined in any one of items 21-26;
   the combination as defined in any one of items 27-36; and/or
   the kit as defined in any one of items 37-42.

The invention, and in particular the items outlined above, are described in more detail below.

### Definitions

Unless otherwise defined herein, scientific and technical terms used in the present application shall have the meanings that are commonly understood by those of ordinary skill in the art. Further, unless otherwise required by context, nomenclatures used herein, and techniques of cell and tissue culture are those well-known and commonly used in the art.

Such techniques are fully explained in the literature, such as Owen et al. (Kuby Immunology, 7^{th}, edition, 2013—W. H. Freeman) and Sambrook et al. (Molecular cloning: A laboratory manual 4th edition, Cold Spring Harbor Laboratory Press—Cold Spring Harbor, N.Y., USA, 2012).

Nevertheless, with respect to the use of different terms throughout the current specification, the following definitions more particularly apply.

The term “(poly)peptide” as used herein refers to a peptide and/or to a polypeptide. The terms “peptide”, “polypeptide”, “protein” and variations of these terms refer to peptides, oligopeptides, polypeptides, or proteins comprising at least two amino acids joined to each other preferably by a normal peptide bond, or, alternatively, by a modified peptide bond, such as for example in the cases of isosteric peptides. In particular, the terms “peptide”, “polypeptide” and “protein” refer to a sequential chain of amino acids of any length linked together via peptide bonds (—NHCO—). Peptides, polypeptides and proteins can play a structural and/or functional role in a cell in vitro and/or in vivo. The terms “peptide”, “polypeptide”, “protein” preferably encompass amino acids chains in size ranging from 2 to at least about 1000 amino acid residues. The term “peptide” preferably encompasses herein amino acid chains in size of less than about 30 amino acids, while the terms “polypeptide” and “protein” preferably encompass amino acid chains in size of at least 30 amino acids. The terms “polypeptide” and “protein” are used herein interchangeably. In a preferred embodiment, the terms “peptide”, “polypeptide”, “protein” also include “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide or protein can comprise amino acids other than the 20 amino acids defined by the genetic code in addition to these amino acids, or it can be composed of amino acids other than the 20 amino acids defined by the genetic code. In particular, a peptide, polypeptide or protein in the context of the present invention can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, “protein” in the context of the present invention in particular also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62). Accordingly, the terms “peptide”, “polypeptide”, “protein” preferably include for example lipopeptides, lipoproteins, glycopeptides, glycoproteins and the like.

In a preferred embodiment, a (poly)peptide or protein is a “classical” (poly)peptide or protein, whereby a “classical” (poly)peptide or protein is typically composed of amino acids selected from the 20 amino acids defined by the genetic code, linked to each other by a normal peptide bond.

As well-known in the art, peptides, polypeptides and proteins can be encoded by nucleic acids. The terms “nucleic acid”, “nucleic acid molecule”, “nucleic acid sequence”, “polynucleotide”, “nucleotide sequence” are used herein interchangeable and refer to a precise succession of natural nucleotides (e.g., A, T, G, C and U), or synthetic nucleotides, i.e. to a chain of at least two nucleotides. In particular, the terms “nucleic acid”, “nucleic acid molecule”, “nucleic acid sequence”, “polynucleotide”, “nucleotide sequence” refer to DNA or RNA. Nucleic acids preferably comprise single stranded, double stranded or partially double stranded DNA or RNA, preferably selected from genomic DNA, cDNA, ribosomal DNA, and the transcription product of said DNA, such as RNA. Preferred examples of nucleic acids include rRNA, mRNA; antisense DNA, antisense RNA; complimentary RNA and/or DNA sequences, ribozyme, (complementary) RNA/DNA sequences with or without expression elements, a vector; a mini-gene, gene fragments, regulatory elements, promoters, and combinations thereof. Further preferred examples of nucleic acid (molecules) and/or polynucleotides include, e.g., a recombinant polynucleotide, a vector, an oligonucleotide, an RNA molecule such as an rRNA, an mRNA, or a tRNA, or a DNA molecule as described above. It is thus preferred that the nucleic acid (molecule) is a DNA molecule or an RNA molecule; preferably selected from genomic DNA; cDNA; rRNA; mRNA; antisense DNA; antisense RNA; complementary RNA and/or DNA sequences; RNA and/or DNA sequences with or without expression elements, regulatory elements, and/or promoters; a vector; and combinations thereof. It is within the skill of the person in the art to determine nucleotide sequences which can encode a specific amino acid sequence.

The (poly)peptides and/or nucleic acids according to the invention may be prepared by any known method in the art including, but not limited to, any synthetic method, any recombinant method, any ex vivo generation method and the like, and any combination thereof. Such techniques are fully explained in the literature as mentioned above.

The term “immunogenic compound” refers to a compound comprising a (poly)peptide according to the present invention. An “immunogenic compound” is able to induce, increase or maintain an immunological response against said (poly)peptide in a subject to whom it is administered. In some embodiments, immunogenic compounds comprise at least one (poly)peptide, or alternatively at least one compound comprising such an (poly)peptide, linked to a protein, which encompasses a carrier protein.

A “carrier protein” is usually a protein, which is able to transport a cargo, such as the (poly)peptide according to the present invention. For example, the carrier protein may transport its cargo across a membrane. In the context of the present invention, a carrier protein in particular (also) encompasses a peptide or a polypeptide that is able to elicit an immune response against the (poly)peptide that is linked thereto. Carrier proteins are known in the art.

Alternatively such carrier peptide or polypeptide may be co-administered in the form of immune adjuvant.

Preferably, the (poly)peptide as described herein may be co-administrated or linked, for example by covalent or non-covalent bond, to a protein/peptide having immuno-adjuvant properties, such as providing stimulation of CD4+ Th1 cells. While the (poly)peptide as described herein preferably binds to MHC class I, CD4+ helper epitopes may be additionally used to provide an efficient immune response. Th1 helper cells are able to sustain efficient dendritic cell (DC) activation and specific CTL activation by secreting interferon-gamma (IFN-γ), tumor necrosis factor-alpha (TNF-α) and interleukine-2 (IL-2) and enhancing expression of costimulatory signal on DCs and T cells (Galaine et al., Interest of Tumor-Specific CD4 T Helper 1 Cells for Therapeutic Anticancer Vaccine. Vaccines (Basel). 2015 Jun. 30; 3(3):490-502).

For example, the adjuvant peptide/protein may preferably be distinct from the (poly)peptide according to the present invention. Preferably, the adjuvant peptide/protein is capable of recalling immune memory or provides a non-specific help or could be a specific helper peptide. Several helper peptides have been described in the literature for providing a nonspecific T cell help, such as tetanus helper peptide, keyhole limpet hemocyanin peptide or PADRE peptide (Adotévi et al., Targeting antitumor CD4 helper T cells with universal tumor-reactive helper peptides derived from telomerase for cancer vaccine. Hum Vaccin Immunother. 2013 May; 9(5):1073-7, Slingluff C L, The present and future of peptide vaccines for cancer: single or multiple, long or short, alone or in combination? Cancer J. 2011 September-October; 17(5):343-50). Accordingly, tetanus helper peptide, keyhole limpet hemocyanin peptide and PADRE peptide are preferred examples of such adjuvant peptide/proteins. In particular, the antigenic peptide as described herein, or a polypeptide comprising the said antigenic peptide, may be linked, for example by covalent or non-covalent bond, to the HHD-DR3 peptide of sequence MAKTIAYDEEARRGLERGLN (SEQ ID NO: 266). This peptide represents another example of a helper peptide (having immuno-adjuvant properties), which is preferred in the context of the present invention. Another preferred example is h-pAg T13L (sequence: TPPAYRPPNAPIL; SEQ ID NO: 280; Bhasin M, Singh H, Raghava G P (2003) MHCBN: a comprehensive database of MHC binding and non-binding peptides. Bioinformatics 19: 665-666). Further examples of preferred helper peptides include the UCP2 peptide (for example as described in WO 2013/135553 A1 or in Dosset M, Godet Y, Vauchy C, Beziaud L, Lone Y C, Sedlik C, Liard C, Levionnois E, Clerc B, Sandoval F, Daguindau E, Wain-Hobson S, Tartour E, Langlade-Demoyen P, Borg C, Adotévi O: Universal cancer peptide-based therapeutic vaccine breaks tolerance against telomerase and eradicates established tumor. Clin Cancer Res. 2012 Nov. 15; 18(22):6284-95. doi: 10.1158/1078-0432.CCR-12-0896. Epub 2012 Oct. 2) and the BIRC5 peptide (for example as described in EP2119726 A1 or in Widenmeyer M, Griesemann H, Stevanović S, Feyerabend S, Klein R, Attig S, Hennenlotter J, Wernet D, Kuprash D V, Sazykin A Y, Pascolo S, Stenzl A, Gouttefangeas C, Rammensee H G: Promiscuous survivin peptide induces robust CD4+ T-cell responses in the majority of vaccinated cancer patients. Int J Cancer. 2012 Jul. 1; 131(1):140-9. doi: 10.1002/ijc.26365. Epub 2011 Sep. 14). The most preferred helper peptide is the UCP2 peptide (amino acid sequence: KSVWSKLQSIGIRQH; SEQ ID NO: 281, for example as described in WO 2013/135553 A1 or in Dosset M, Godet Y, Vauchy C, Beziaud L, Lone Y C, Sedlik C, Liard C, Levionnois E, Clerc B, Sandoval F, Daguindau E, Wain-Hobson S, Tartour E, Langlade-Demoyen P, Borg C, Adotévi O: Universal cancer peptide-based therapeutic vaccine breaks tolerance against telomerase and eradicates established tumor. Clin Cancer Res. 2012 Nov. 15; 18(22):6284-95. doi: 10.1158/1078-0432.CCR-12-0896. Epub 2012 Oct. 2).

A composition as defined herein, which further comprises one or more immuno-adjuvant substances, may also be termed an “immunogenic composition” or in some embodiments a “vaccine composition” in the present specification. As used herein, the term “immunogenic composition” refers to a composition that is able to induce or maintain an immune response, in particular which induces an immune response, when it is administered to a mammal, and especially when it is administered to a human individual.

By “pharmaceutically acceptable excipient”, it is meant herein a compound of pharmaceutical grade which improves the delivery, stability or bioavailability of an active agent, and can be metabolized by, and is non-toxic to, a subject to whom it is administered. Preferred excipients according to the invention include any of the excipients commonly used in pharmaceutical products, such as, for example, water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable excipients may further comprise minor amounts of auxiliary substances such as wetting or emulsifying agents, or preservatives.

By “vaccine”, it is meant herein a composition capable of stimulating the immune system of a living organism so that protection against a harmful antigen is provided, either through prophylaxis or through therapy. Prophylactic vaccines are preferred.

According to the different aspects and embodiments of the invention described herein, a “subject” or “host” preferably refers to a mammal, and most preferably to a human being. Said subject may have, been suspected of having, or be at risk of developing fibrosis, an autoimmune disease/disorder and/or an inflammatory disease.

As used herein, the term “preventing”, “prevention”, “prophylaxis” or “prevent” generally means to avoid or minimize the onset or development of a disease or condition before its onset, while the term “treating, “treatment” or “treat” encompasses reducing, ameliorating or curing a disease or condition (or symptoms of a disease or condition) after its onset. The term “preventing” encompasses “reducing the likelihood of occurrence of” or «reducing the likelihood of reoccurrence».

An “effective amount” or “effective dose” as used herein is an amount which provides the desired effect. For therapeutic purposes, an effective amount is an amount sufficient to provide a beneficial or desired clinical result. The preferred effective amount for a given application can be easily determined by the skilled person taking into consideration, for example, the size, age, weight of the subject, the type of disease/disorder to be prevented or treated, and the amount of time since the disease/disorder began. In the context of the present invention, in terms of prevention or treatment, an effective amount of the composition is an amount that is sufficient to induce a humoral and/or cell-mediated immune response directed against the disease/disorder.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x ±10%.

Additional definitions are provided throughout the specification.

The present invention may be understood more readily by reference to the following detailed description, including preferred embodiments of the invention, and examples included herein.

### DETAILED DESCRIPTION

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

(Poly)Peptide Comprising an IL13RA2 Epitope or a Sequence Variant Thereof

In a first aspect the present invention provides a (poly)peptide comprising or consisting of an epitope of IL13RA2 or a sequence variant thereof having at least 70% (preferably at least 75%, more preferably at least 80%, even more preferably at least 85%, and most preferably at least 90%) sequence identity for use in prevention and/or treatment of fibrosis, an autoimmune disease and/or an inflammatory disease. Accordingly, the present invention also provides a method for ameliorating, reducing, preventing and/or treating fibrosis, an autoimmune disease and/or an inflammatory disease in a subject, comprising administering to the subject a (poly)peptide comprising an epitope of IL13RA2 or a sequence variant thereof having at least 70% (preferably at least 75%, more preferably at least 80%, even more preferably at least 85%, and most preferably at least 90%) sequence identity.

The present inventors have surprisingly found that fibrosis, an autoimmune disease and/or an inflammatory disease can be prevented or treated by antigen-based immunotherapy targeting IL13RA2, namely, by administration of an epitope of IL13RA2 or a sequence variant thereof.

The term “epitope”, as used herein, refers to a peptide, which can be recognized by the immune system. In general, an “epitope” (also known as “antigenic determinant”), is the part (or fragment) of an antigen that is recognized by the immune system, in particular by antibodies, T cell receptors, and/or B cell receptors. Thus, one antigen has at least one epitope, i.e. a single antigen has one or more epitopes. An “antigen” typically serves as a target for the receptors of an adaptive immune response, in particular as a target for antibodies, T cell receptors, and/or B cell receptors. In the context of the present invention the antigen is interleukin-13 receptor alpha 2 (IL13RA2). Human interleukin-13 receptor alpha 2 (IL13RA2) is preferred. The sequence of human IL13RA2 is shown in the following:

[SEQ ID NO: 275]
MAFVCLAIGCLYTFLISTTFGCTSSSDTEIKVNPPQDFEIVDPGYLGYLY
LQWQPPLSLDHFKECTVEYELKYRNIGSETWKTIITKNLHYKDGFDLNKG
IEAKIHTLLPWQCTNGSEVQSSWAETTYWISPQGIPETKVQDMDCVYYNW
QYLLCSWKPGIGVLLDTNYNLFYWYEGLDHALQCVDYIKADGQNIGCRFP
YLEASDYKDFYICVNGSSENKPIRSSYFTFQLQNIVKPLPPVYLTFTRES
SCEIKLKWSIPLGPIPARCEDYEIEIREDDTTLVTATVENETYTLKTTNE
TRQLCFVVRSKVNIYCSDDGIWSEWSDKQCWEGEDLSKKTLLRFWLPFGF
ILILVIFVTGLLLRKPNTYPKMIPEFFCDT

Accordingly, a preferred (poly)peptide for use according to the present invention comprises an epitope of human IL13RA2. For example, a preferred (poly)peptide for use according to the present invention may comprise an amino acid sequence according to SEQ ID NO: 275 or a fragment thereof comprising or consisting of an epitope or a sequence variant of such a fragment. As used herein, a “fragment” of an antigen comprises at least 5 consecutive amino acids of the antigen, preferably at least 6 consecutive amino acids of the antigen, more preferably at least 7 consecutive amino acids of the antigen, even more preferably at least 8 consecutive amino acids of the antigen and most preferably at least 9 consecutive amino acids of the antigen. A “sequence variant” is as defined herein, namely a sequence variant has an (amino acid) sequence which is at least 70% (preferably at least 75%, more preferably at least 80%, even more preferably at least 85%, most preferably at least 90%) identical to the reference sequence. A “functional” sequence variant means in the context of an antigen/antigen fragment/epitope, that the function of the epitope(s), e.g. comprised by the antigen (fragment), is not impaired or abolished.

In the context of the present invention, the term “epitope” is mainly used to designate T cell epitopes, which are presented on the surface of an antigen-presenting cell, where they are bound to Major Histocompatibility Complex (MHC). T cell epitopes presented by MHC class I molecules are typically, but not exclusively, peptides between 8 and 12 amino acids in length, whereas MHC class II molecules present longer peptides, generally, but not exclusively, between 12 and 25 amino acids in length. Preferably, the epitope of IL13RA2 or the sequence variant thereof has a length of 8-12 amino acids, more preferably of 8-10 amino acids and most preferably of 9 or 10 amino acids.

Several epitopes of IL13Ralpha2 are known to the skilled person. For example, suitable epitopes of IL13RA2 are known to the skilled person from the known use of IL13RA2 as tumor antigen/tumor-associated antigen, e.g. from literature referring to IL13RA2 epitopes in the context of cancer immunotherapy. Such epitopes can also be identified by using cancer/tumor epitope databases, e.g. from van der Bruggen P, Stroobant V, Vigneron N, Van den Eynde B. Peptide database: T cell-defined tumor antigens. Cancer Immun 2013; URL: http://www.cancerimmunity.org/peptide/, wherein human tumor antigens recognized by CD4+ or CD8+ T cells are classified into four major groups on the basis of their expression pattern, or from the database “Tantigen” (TANTIGEN version 1.0, Dec. 1, 2009; developed by Bioinformatics Core at Cancer Vaccine Center, Dana-Farber Cancer Institute; URL: http://cvc.dfci.harvard.edu/tadb/).

Preferred examples of epitopes of IL13RA2 have an amino acid sequence as set forth in any one of SEQ ID NOs 243-265 and 276-278 as shown in Table 1 below.

TABLE 1
Preferred examples of human IL13RA2 epitopes

| SEQ ID NO. | IL13 RA2 epitope sequence |
|---|---|
| 243 | AIGCLYTFL |
| 244 | ASDYKDEYI |
| 245 | CLYTFLIST |
| 246 | CSDDGIWSE |
| 247 | EASDYKDFY |
| 248 | ETWKTIITK |
| 249 | FLISTTFGC |
| 250 | FVTGLLLRK |
| 251 | GLDHALQCV |
| 252 | ILVIFVTGL |
| 253 | KVQDMCVYY |
| 254 | LDTNYNLFY |
| 255 | LLCSWKPGI |
| 256 | LQWQPPLSL |
| 257 | NIVKPLPPV |
| 258 | NLFYWYEGL |
| 259 | QSSWAETTY |
| 260 | RNIGSETWK |
| 261 | VCLAIGCLY |
| 262 | VENETYTLK |
| 263 | WLPFGFILI |
| 264 | WQYLLCSWK |
| 265 | WSDKQCWEG |
| 276 | WLPFGFILIL |
| 277 | VLLDTNYNL |
| 278 | GLDHALQCV |

Of those exemplified IL13RA2 epitopes, any one of amino acid sequences as set forth in SEQ ID NOs 245, 249, 251, 252, 255, 257, 263 and 276-278 is more preferred. The most preferred examples of epitopes of IL13RA2 have an amino acid sequence as set forth in SEQ ID NO: 258, 277 or 278. Accordingly, an epitope of IL13RA2 having an amino acid sequence as set forth in SEQ ID NO: 258 is particularly preferred. Moreover, an epitope of IL13RA2 having an amino acid sequence as set forth in SEQ ID NO: 277 is particularly preferred. Moreover, an epitope of IL13RA2 having an amino acid sequence as set forth in SEQ ID NO: 278 is particularly preferred.

Accordingly, it is preferred that the (poly)peptide for use according to the present invention comprises or consists of an epitope of IL13RA2 having an amino acid sequence as set forth in any one of SEQ ID NOs 243-265 and 276-278, preferably as set forth in SEQ ID NOs 245, 249, 251, 252, 255, 257, 263 and 276-278, and more preferably as set forth in SEQ ID NO: 258, 277 or 278.

It is also preferred that the (poly)peptide for use according to the present invention comprises or consists of a sequence variant of a (human) IL13RA2 epitope as described herein. For example the (poly)peptide for use according to the present invention may comprise or consist of a sequence variant of an amino acid as set forth in any one of SEQ ID NOs 243-265 and 276-278, preferably as set forth in SEQ ID NOs 245, 249, 251, 252, 255, 257, 263 and 276-278 and most preferably as set forth in SEQ ID NO: 258, 277 or 278.

As used herein, a “sequence variant” is similar, but contains at least one alteration, in comparison to a (human) IL13RA2 epitope (reference sequence). A “sequence variant” may be a recombinant sequence variant (which does not occur in nature), for example which is designed in vitro, e.g. by mutating a (human) IL13RA2 epitope (reference sequence). A “sequence variant” may also be a naturally occurring sequence variant, such as a naturally occurring peptide or a fragment of a naturally occurring protein (for example it may be found in a species other than human, such as a microbiota sequence variant as described below), which shares sequence identity with a (human) IL13RA2 epitope. Such a naturally occurring protein or peptide (which comprises a sequence variant of a (human) IL13RA2 epitope) may be an IL13RA2 homologue or it may be unrelated to IL13RA2. Preferably, the sequence variant has a length of at least 5 amino acids, more preferably at least 6 amino acids, even more preferably at least 7 amino acids, and most preferably at least 8 amino acids. For example, the “sequence variant” may have a length of 9 or 10 amino acids. Preferably, the sequence variant has a length of 8-12 amino acids, more preferably the sequence variant has a length of 8-10 amino acids. Peptides having such a length can bind to MHC (major histocompatibility complex) class I (MHC I), which is crucial for a cytotoxic T-lymphocyte (CTL) response. It is also preferred that the sequence variant has a length of 13-24 amino acids. Peptides having such a length can bind to MHC (major histocompatibility complex) class II (MHC II), which is crucial for a CD4+ T-cell (T helper cell) response.

In general, the term “sequence variant”, as used herein, i.e. throughout the present application, refers to a sequence which is similar (meaning in particular at least 50% sequence identity, see below), but not (100%) identical, to a reference sequence (such as (human) IL13RA2 or an epitope or fragment thereof). Accordingly, a sequence variant contains at least one alteration in comparison to a reference sequence. For example, in a sequence variant one or more of the amino acids or nucleotides of the reference sequence is deleted or substituted, or one or more amino acids or nucleotides are inserted into the sequence of the reference sequence. Therefore, the “sequence variant” is similar, but contains at least one alteration, in comparison to its reference sequence, which is an IL13RA2 epitope sequence. Preferably, a sequence variant shares, in particular over the whole length of the sequence, at least 60%, preferably at least 70%, more preferably at least 75%, more preferably at least 80%, even more preferably at least 85%, and most preferably at least 90% sequence identity with a reference sequence (such as the IL13RA2 epitope sequence).

Preferably, a sequence variant preserves the specific function of the reference sequence. In the context of the present invention, this function is the functionality as an “epitope”, i.e. it can be recognized by the immune system, in particular by antibodies, T cell receptors, and/or B cell receptors and, preferably, it can elicit an immune response. Accordingly, is preferred that the (poly)peptide for use according to the present invention, in particular the epitope of IL13RA2 or the sequence variant thereof, is immunogenic. In other words, the (poly)peptide, in particular the epitope of IL13RA2 or the sequence variant thereof, is preferably capable of eliciting an immune response.

The term “sequence variant” includes nucleotide sequence variants and amino acid sequence variants. For example, an amino acid sequence variant has an altered sequence in which one or more of the amino acids is deleted or substituted in comparison to the reference sequence, or one or more amino acids are inserted in comparison to the reference amino acid sequence. As a result of the alterations, the amino acid sequence variant has an amino acid sequence which is at least 50%, preferably at least 60%, more preferably at least 70%, more preferably at least 75%, even more preferably at least 80%, even more preferably at least 85%, and most preferably at least 90% identical to the reference sequence. For example, variant sequences which are at least 90% identical have no more than 10 alterations (i.e. any combination of deletions, insertions or substitutions) per 100 amino acids of the reference sequence.

In the context of the present invention, an amino acid sequence “sharing a sequence identity” of at least, for example, 70% to a query amino acid sequence of the present invention, is intended to mean that the sequence of the subject amino acid sequence is identical to the query sequence except that the subject amino acid sequence may include up to three amino acid alterations per each 10 amino acids of the query amino acid sequence. In other words, to obtain an amino acid sequence having a sequence of at least 70% identity to a query amino acid sequence, up to 30% (3 of 10) of the amino acid residues in the subject sequence may be inserted or substituted with another amino acid or deleted, preferably within the above definitions of variants or fragments. The same, of course, also applies similarly to nucleic acid sequences.

For (amino acid or nucleic acid) sequences without exact correspondence, a “% identity” of a first sequence (e.g., the sequence variant) may be determined with respect to a second sequence (e.g., the reference sequence). In general, the two sequences to be compared may be aligned to give a maximum correlation between the sequences. This may include inserting “gaps” in either one or both sequences, to enhance the degree of alignment. A % identity may then be determined over the whole length of each of the sequences being compared (so-called “global alignment”), that is particularly suitable for sequences of the same or similar length, or over shorter, defined lengths (so-called “local alignment”), that is more suitable for sequences of unequal length.

Methods for comparing the identity (sometimes also referred to as “similarity” or “homology”) of two or more sequences are well known in the art. The percentage to which two (or more) sequences are identical can e.g. be determined using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs, e.g. BLAST or NBLAST program (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 183, 63-98; Pearson and Lipman (1988), Proc. Natl. Acad. Sci. U. S. A 85, 2444-2448.). Sequences which are identical to other sequences to a certain extent can be identified by these programmes. Furthermore, programs available in the Wisconsin Sequence Analysis Package, version 9.1 (Devereux et al., 1984, Nucleic Acids Res., 387-395), for example the programs BESTFIT and GAP, may be used to determine the identity between two polynucleotides and the % identity and the % homology or identity between two polypeptide sequences. BESTFIT uses the “local homology” algorithm of (Smith and Waterman (1981), J. Mol. Biol. 147, 195-197.) and finds the best single region of similarity between two sequences.

Preferably, the sequence variant differs from the (human) IL13RA2 epitope (only) in primary and/or secondary anchor residues for MHC molecules. More preferably, the sequence variant differs from the (human) IL13RA2 epitope (only) in that it comprises amino acid substitutions (only) in primary and/or secondary anchor residues for MHC molecules. Anchor residues for the HLA subtypes are known in the art, and can be defined by large throughput analysis of structural data of existing p-HLA complexes in the Protein Data Bank. Moreover, anchor motifs for MHC subtypes can also be found in IEDB (URL: www.iedb.org; browse by allele) or in SYFPEITHI (URL: http://www.syfpeithi.de/). For example, for a 9 amino acid size HLA.A2.01 peptide, the peptide primary anchor residues, providing the main contact points, are located at residue positions P1, P2 and P9.

Accordingly, it is preferred that the core sequence of the sequence variant is identical with the core sequence of the (human) IL13RA2 epitope, wherein the “core sequence” consists of all amino acids except the (at least) three most N-terminal and the (at least) three most C-terminal amino acids of the (human) IL13RA2 epitope. Accordingly, it is preferred that any alterations in the sequence variant in comparison to the (human) IL13RA2 epitope are preferably located within the three N-terminal and/or within the three C-terminal amino acids of the (human) IL13RA2 epitope, but not in the “core sequence” of the (human) IL13RA2 epitope (amino acids in the middle of the of the (human) IL13RA2 epitope sequence). This does not mean that all three N-terminal and/or C-terminal amino acids of a (human) IL13RA2 epitope must be altered, but only that those are the preferred amino acid positions, where an amino acid may be altered. For example, in IL13RA2 epitope of nine amino acids, the three middle amino acids may represent the core sequence and alterations may preferably only occur at any of the three N-terminal and the three C-terminal amino acid positions.

More preferably, the core sequence of the (human) IL13RA2 epitope consists of all amino acids except the two most N-terminal and the two most C-terminal amino acids of the (human) IL13RA2 epitope. For example, in a (human) IL13RA2 epitope of nine amino acids, the five middle amino acids may represent the core sequence and alterations may preferably only occur at any of the two N-terminal and the two C-terminal amino acid positions of the (human) IL13RA2 epitope.

It is also preferred that the core sequence of the (human) IL13RA2 epitope consists of all amino acids except the most N-terminal and the most C-terminal amino acid of the (human) IL13RA2 epitope. For example, in a (human) IL13RA2 epitope of nine amino acids, the seven middle amino acids may represent the core sequence and alterations may preferably only occur at the N-terminal position (P1) and the C-terminal amino acid position (P9) of the (human) IL13RA2 epitope.

Most preferably, the core sequence of the (human) IL13RA2 epitope consists of all amino acids except the two most N-terminal amino acids and the most C-terminal amino acid of the (human) IL13RA2 epitope. For example, in a (human) IL13RA2 epitope of nine amino acids, the six middle amino acids may represent the core sequence and alterations may preferably only occur at any of the two N-terminal positions (P1 and P2) and the C-terminal amino acid position (P9) of the (human) IL13RA2 epitope.

It is particularly preferred that the sequence variant, e.g. having a length of nine amino acids, comprises at position 1 (P1; the most N-terminal amino acid position) a phenylalanine (F) or a lysine (K). Moreover, it is preferred that the sequence variant, e.g. having a length of nine amino acids, comprises at position 2 (P2) a leucine (L) or a methionine (M). Moreover, it is preferred that the sequence variant, e.g. having a length of nine amino acids, comprises at position 9 (P9) a valine (V) or a leucine (L). Most preferably, the sequence variant, e.g. having a length of nine amino acids, comprises at position 1 (P1; the most N-terminal amino acid position) a phenylalanine (F) or a lysine (K), at position 2 (P2) a leucine (L) or a methionine (M) and/or at position 9 (P9) a valine (V) or a leucine (L).

Moreover, amino acid substitutions, in particular at positions other than the anchor position(s) for MHC molecules (e.g., P1, P2 and P9 for MHC-I subtype HLA.A2.01), are preferably conservative amino acid substitutions. Examples of conservative substitutions include substitution of one aliphatic residue for another, such as Ile, Val, Leu, or Ala for one another; or substitutions of one polar residue for another, such as between Lys and Arg; Glu and Asp; or Gln and Asn. Other such conservative substitutions, for example, substitutions of entire regions having similar hydrophobicity properties, are well known (Kyte and Doolittle, 1982, J. Mol. Biol. 157(1):105-132). Examples of conservative amino acid substitutions are presented in Table 2 below:

TABLE 2

| Original residues | Examples of substitutions |
|---|---|
| Ala (A) | Val, Leu, Ile, Gly |
| Arg (R) | His, Lys |
| Asn (N) | Gln |
| Asp (D) | Glu |
| Cys (C) | Ser |
| Gln (Q) | Asn |
| Glu (E) | Asp |
| Gly (G) | Pro, Ala |
| His (H) | Lys, Arg |
| Ile (I) | Leu, Val, Met, Ala, Phe |
| Leu (L) | Ile, Val, Met, Ala, Phe |
| Lys (K) | Arg, His |
| Met (M) | Leu, Ile, Phe |
| Phe (F) | Leu, Val, Ile, Tyr, Trp, Met |
| Pro (P) | Ala, Gly |
| Ser (S) | Thr |
| Thr (T) | Ser |
| Trp (W) | Tyr, Phe |
| Tyr (Y) | Trp, Phe |
| Val (V) | Ile, Met, Leu, Phe, Ala |

Preferred examples of sequence variants are sequence variants of a (human) IL13RA2 epitope having an amino acid sequences as set forth in any one of SEQ ID NOs 243-265 and 276-278.

Accordingly, it is preferred that the (poly)peptide for use according to the present invention comprises an amino acid sequence as set forth in any one of SEQ ID NOs 1-242, 267-274 and 279. The amino acid sequences as set forth in any one of SEQ ID NOs 1-242, 267-274 and 279 represent preferred examples of sequence variants of human IL13RA2 epitopes. As shown in the examples herein, the exemplified sequence variants allow the raise of a strong immune response against themselves, and most importantly, allow the raise of a strong immune response against peptides having amino acid similarity therewith which are comprised in the IL13RA2 antigen.

Preferred examples of sequence variants are listed in Table 3 below, which also provides information regarding the corresponding reference sequence in human IL13RA2, and HLA class. The sequence IDs SEQ ID NO: 1 to 242, 267-274 and 279 refer to the sequence variants.

TABLE 3
Examples of preferred sequence variants of human IL13RA2 epitopes

| SEQ ID NO. | | Reference sequence in | |
|---|---|---|---|
| sequence variant | Sequence variant | human IL13RA2 | HLA |
| 1 | ALGCLYTFV | AIGCLYTFL | HLA-A*02: 01 |
| 2 | AMGCLYTFI | AIGCLYTFL | HLA-A*02: 01 |
| 3 | AMGCLYTFV | AIGCLYTFL | HLA-A*02: 01 |
| 9 | FIGCLYTFI | AIGCLYTFL | HLA-A*02: 01 |
| 10 | FIGCLYTFL | AIGCLYTFL | HLA-A*02: 01 |
| 11 | FIGCLYTFV | AIGCLYTFL | HLA-A*02: 01 |
| 24 | FLGCLYTFI | AIGCLYTFL | HLA-A*02: 01 |
| 25 | FLGCLYTFL | AIGCLYTFL | HLA-A*02: 01 |
| 26 | FLGCLYTFV | AIGCLYTFL | HLA-A*02: 01 |
| 57 | FMGCLYTFI | AIGCLYTFL | HLA-A*02: 01 |
| 58 | FMGCLYTFL | AIGCLYTFL | HLA-A*02: 01 |
| 59 | FMGCLYTFV | AIGCLYTFL | HLA-A*02: 01 |
| 111 | WLGCLYTFI | AIGCLYTFL | HLA-A*02: 01 |
| 112 | WLGCLYTFL | AIGCLYTFL | HLA-A*02: 01 |
| 113 | WLGCLYTFV | AIGCLYTFL | HLA-A*02: 01 |
| 130 | WMGCLYTFI | AIGCLYTFL | HLA-A*02: 01 |
| 131 | WMGCLYTFL | AIGCLYTFL | HLA-A*02: 01 |
| 132 | WMGCLYTFV | AIGCLYTFL | HLA-A*02: 01 |
| 158 | YIGCLYTFI | AIGCLYTFL | HLA-A*02: 01 |
| 159 | YIGCLYTFL | AIGCLYTFL | HLA-A*02: 01 |
| 160 | YIGCLYTFV | AIGCLYTFL | HLA-A*02: 01 |
| 171 | YLGCLYTFI | AIGCLYTFL | HLA-A*02: 01 |
| 172 | YLGCLYTFL | AIGCLYTFL | HLA-A*02: 01 |
| 173 | YLGCLYTFV | AIGCLYTFL | HLA-A*02: 01 |
| 204 | YMGCLYTFI | AIGCLYTFL | HLA-A*02: 01 |
| 205 | YMGCLYTFL | AIGCLYTFL | HLA-A*02: 01 |
| 206 | YMGCLYTFV | AIGCLYTFL | HLA-A*02: 01 |
| 89 | FSDYKDFYI | ASDYKDFYI | HLA-A*01: 01 |
| 90 | FSDYKDFYL | ASDYKDFYI | HLA-A*01: 01 |
| 91 | FSDYKDFYV | ASDYKDFYI | HLA-A*01: 01 |
| 153 | WSDYKDFYI | ASDYKDFYI | HLA-A*01: 01 |
| 154 | WSDYKDFYL | ASDYKDFYI | HLA-A*01: 01 |
| 155 | WSDYKDFYV | ASDYKDFYI | HLA-A*01: 01 |
| 237 | YSDYKDFYI | ASDYKDFYI | HLA-A*01: 01 |
| 238 | YSDYKDFYL | ASDYKDFYI | HLA-A*01: 01 |
| 239 | YSDYKDFYV | ASDYKDFYI | HLA-A*01: 01 |
| 4 | CMYTFLISV | CLYTFLIST | HLA-A*02: 01 |
| 42 | FLYTFLISI | CLYTFLIST | HLA-A*02: 01 |
| 43 | FLYTFLISL | CLYTFLIST | HLA-A*02: 01 |
| 44 | FLYTFLIST | CLYTFLIST | HLA-A*02: 01 |
| 45 | FLYTFLISV | CLYTFLIST | HLA-A*02: 01 |
| 78 | FMYTFLISI | CLYTFLIST | HLA-A*02: 01 |
| 79 | FMYTFLISL | CLYTFLIST | HLA-A*02: 01 |
| 80 | FMYTFLIST | CLYTFLIST | HLA-A*02: 01 |
| 81 | FMYTFLISV | CLYTFLIST | HLA-A*02: 01 |
| 123 | WLYTFLISV | CLYTFLIST | HLA-A*02: 01 |
| 144 | WMYTFLISI | CLYTFLIST | HLA-A*02: 01 |
| 145 | WMYTFLISL | CLYTFLIST | HLA-A*02: 01 |
| 146 | WMYTFLISV | CLYTFLIST | HLA-A*02: 01 |
| 190 | YLYTFLISI | CLYTFLIST | HLA-A*02: 01 |
| 191 | YLYTFLISL | CLYTFLIST | HLA-A*02: 01 |
| 192 | YLYTFLIST | CLYTFLIST | HLA-A*02: 01 |
| 193 | YLYTFLISV | CLYTFLIST | HLA-A*02: 01 |
| 226 | YMYTFLISI | CLYTFLIST | HLA-A*02: 01 |
| 227 | YMYTFLISL | CLYTFLIST | HLA-A*02: 01 |
| 228 | YMYTFLIST | CLYTFLIST | HLA-A*02: 01 |
| 229 | YMYTFLISV | CLYTFLIST | HLA-A*02: 01 |
| 5 | CSDDGIWSI | CSDDGIWSE | HLA-A*01: 01 |
| 6 | CSDDGIWSL | CSDDGIWSE | HLA-A*01: 01 |
| 7 | CSDDGIWSV | CSDDGIWSE | HLA-A*01: 01 |
| 16 | FLDDGIWSI | CSDDGIWSE | HLA-A*01: 01 |
| 17 | FLDDGIWSL | CSDDGIWSE | HLA-A*01: 01 |
| 18 | FLDDGIWSV | CSDDGIWSE | HLA-A*01: 01 |
| 49 | FMDDGIWSI | CSDDGIWSE | HLA-A*01: 01 |
| 50 | FMDDGIWSL | CSDDGIWSE | HLA-A*01: 01 |
| 51 | FMDDGIWSV | CSDDGIWSE | HLA-A*01: 01 |
| 83 | FSDDGIWSI | CSDDGIWSE | HLA-A*01: 01 |
| 84 | FSDDGIWSL | CSDDGIWSE | HLA-A*01: 01 |
| 85 | FSDDGIWSV | CSDDGIWSE | HLA-A*01: 01 |
| 107 | WLDDGIWSI | CSDDGIWSE | HLA-A*01: 01 |
| 108 | WLDDGIWSL | CSDDGIWSE | HLA-A*01: 01 |
| 109 | WLDDGIWSV | CSDDGIWSE | HLA-A*01: 01 |
| 125 | WMDDGIWSI | CSDDGIWSE | HLA-A*01: 01 |
| 126 | WMDDGIWSL | CSDDGIWSE | HLA-A*01: 01 |
| 127 | WMDDGIWSV | CSDDGIWSE | HLA-A*01: 01 |
| 147 | WSDDGIWSI | CSDDGIWSE | HLA-A*01: 01 |
| 148 | WSDDGIWSL | CSDDGIWSE | HLA-A*01: 01 |
| 149 | WSDDGIWSV | CSDDGIWSE | HLA-A*01: 01 |
| 163 | YLDDGIWSI | CSDDGIWSE | HLA-A*01: 01 |
| 164 | YLDDGIWSL | CSDDGIWSE | HLA-A*01: 01 |
| 165 | YLDDGIWSV | CSDDGIWSE | HLA-A*01: 01 |
| 196 | YMDDGIWSI | CSDDGIWSE | HLA-A*01: 01 |
| 197 | YMDDGIWSL | CSDDGIWSE | HLA-A*01: 01 |
| 198 | YMDDGIWSV | CSDDGIWSE | HLA-A*01: 01 |
| 231 | YSDDGIWSI | CSDDGIWSE | HLA-A*01: 01 |
| 232 | YSDDGIWSL | CSDDGIWSE | HLA-A*01: 01 |
| 233 | YSDDGIWSV | CSDDGIWSE | HLA-A*01: 01 |
| 8 | FASDYKDFY | EASDYKDFY | HLA-A*01: 01 |
| 32 | FLSDYKDFY | EASDYKDFY | HLA-A*01: 01 |
| 66 | FMSDYKDFY | EASDYKDFY | HLA-A*01: 01 |
| 105 | WASDYKDFY | EASDYKDFY | HLA-A*01: 01 |
| 117 | WLSDYKDFY | EASDYKDFY | HLA-A*01: 01 |
| 137 | WMSDYKDFY | EASDYKDFY | HLA-A*01: 01 |
| 157 | YASDYKDFY | EASDYKDFY | HLA-A*01: 01 |
| 180 | YLSDYKDFY | EASDYKDFY | HLA-A*01: 01 |
| 214 | YMSDYKDFY | EASDYKDFY | HLA-A*01: 01 |
| 37 | FLWKTIITK | ETWKTIITK | HLA-A*03: 01 |
| 73 | FMWKTIITK | ETWKTIITK | HLA-A*03: 01 |
| 93 | FTWKTIITK | ETWKTIITK | HLA-A*03: 01 |
| 140 | WMWKTIITK | ETWKTIITK | HLA-A*03: 01 |
| 185 | YLWKTIITK | ETWKTIITK | HLA-A*03: 01 |
| 221 | YMWKTIITK | ETWKTIITK | HLA-A*03: 01 |
| 241 | YTWKTIITK | ETWKTIITK | HLA-A*03: 01 |
| 27 | FLISTTFGI | FLISTTFGC | HLA-A*02: 01 |
| 28 | FLISTTFGL | FLISTTFGC | HLA-A*02: 01 |
| 29 | FLISTTFGV | FLISIIFGC | HLA-A*02: 01 |
| 60 | FMISTTFGI | FLISTTFGC | HLA-A*02: 01 |
| 61 | FMISTTFGL | FLISTTFGC | HLA-A*02: 01 |
| 62 | FMISTTFGV | FLISTTFGC | HLA-A*02: 01 |
| 114 | WLISTTFGL | FLISTTFGC | HLA-A*02: 01 |
| 115 | WLISTTFGV | FLISTTFGC | HLA-A*02: 01 |
| 133 | WMISTTFGI | FLISTTFGC | HLA-A*02: 01 |
| 134 | WMISTTFGL | FLISTTFGC | HLA-A*02: 01 |
| 135 | WMISTTFGV | FLISTTFGC | HLA-A*02: 01 |
| 174 | YLISTTFGI | FLISTTFGC | HLA-A*02: 01 |
| 175 | YLISIIFGL | FLISTTFGC | HLA-A*02: 01 |
| 176 | YLISTTFGV | FLISTTFGC | HLA-A*02: 01 |
| 207 | YMISTTFGI | FLISTTFGC | HLA-A*02: 01 |
| 208 | YMISTTFGL | FLISTTFGC | HLA-A*02: 01 |
| 209 | YMISTTFGV | FLISTTFGC | HLA-A*02: 01 |
| 67 | FMTGLLLRK | FVTGLLLRK | HLA-A*03: 01 |
| 215 | YMTGLLLRK | FVTGLLLRK | HLA-A*03: 01 |
| 19 | FLDHALQCV | GLDHALQCV | HLA-A*02: 01 |
| 52 | FMDHALQCV | GLDHALQCV | HLA-A*02: 01 |
| 166 | YLDHALQCV | GLDHALQCV | HLA-A*02: 01 |
| 199 | YMDHALQCV | GLDHALQCV | HLA-A*02: 01 |
| 69 | FMVIFVTGV | ILVIFVTGL | HLA-A*02: 01 |
| 217 | YMVIFVTGV | ILVIFVTGL | HLA-A*02: 01 |
| 65 | FMQDMCVYY | KVQDMCVYY | HLA-A*01: 01 |
| 94 | FVQDMCVYY | KVQDMCVYY | HLA-A*01: 01 |
| 156 | WVQDMCVYY | KVQDMCVYY | HLA-A*01: 01 |
| 179 | YLQDMCVYY | KVQDMCVYY | HLA-A*01: 01 |
| 213 | YMQDMCVYY | KVQDMCVYY | HLA-A*01: 01 |
| 242 | YVQDMCVYY | KVQDMCVYY | HLA-A*01: 01 |
| 33 | FLTNYNLFY | LDTNYNLFY | HLA-A*01: 01 |
| 68 | FMTNYNLFY | LDTNYNLFY | HLA-A*01: 01 |
| 95 | LLTNYNLFY | LDTNYNLFY | HLA-A*01: 01 |
| 97 | LMTNYNLFY | LDTNYNLFY | HLA-A*01: 01 |
| 118 | WLTNYNLFY | LDTNYNLFY | HLA-A*01: 01 |
| 138 | WMTNYNLFY | LDTNYNLFY | HLA-A*01: 01 |
| 181 | YLTNYNLFY | LDTNYNLFY | HLA-A*01: 01 |
| 216 | YMTNYNLFY | LDTNYNLFY | HLA-A*01: 01 |
| 13 | FLCSWKPGI | LLCSWKPGI | HLA-A*02: 01 |
| 14 | FLCSWKPGL | LLCSWKPGI | HLA-A*02: 01 |
| 15 | FLCSWKPGV | LLCSWKPGI | HLA-A*02: 01 |
| 46 | FMCSWKPGI | LLCSWKPGI | HLA-A*02: 01 |
| 47 | FMCSWKPGL | LLCSWKPGI | HLA-A*02: 01 |
| 48 | FMCSWKPGV | LLCSWKPGI | HLA-A*02: 01 |
| 106 | WLCSWKPGV | LLCSWKPGI | HLA-A*02: 01 |
| 124 | WMCSWKPGV | LLCSWKPGI | HLA-A*02: 01 |
| 162 | YLCSWKPGV | LLCSWKPGI | HLA-A*02: 01 |
| 194 | YMCSWKPGI | LLCSWKPGI | HLA-A*02: 01 |
| 195 | YMCSWKPGV | LLCSWKPGI | HLA-A*02: 01 |
| 38 | FLWQPPLSI | LQWQPPLSL | HLA-A*02: 01 |
| 39 | FLWQPPLSL | LQWQPPLSL | HLA-A*02: 01 |
| 40 | FLWQPPLSV | LQWQPPLSL | HLA-A*02: 01 |
| 74 | FMWQPPLSI | LQWQPPLSL | HLA-A*02: 01 |
| 75 | FMWQPPLSL | LQWQPPLSL | HLA-A*02: 01 |
| 76 | FMWQPPLSV | LQWQPPLSL | HLA-A*02: 01 |
| 82 | FQWQPPLSV | LQWQPPLSL | HLA-A*02: 01 |
| 96 | LLWQPPLSV | LQWQPPLSL | HLA-A*02: 01 |
| 98 | LMWQPPLSI | LQWQPPLSL | HLA-A*02: 01 |
| 99 | LMWQPPLSL | LQWQPPLSL | HLA-A*02: 01 |
| 100 | LMWQPPLSV | LQWQPPLSL | HLA-A*02: 01 |
| 120 | WLWQPPLSI | LQWQPPLSL | HLA-A*02: 01 |
| 121 | WLWQPPLSL | LQWQPPLSL | HLA-A*02: 01 |
| 122 | WLWQPPLSV | LQWQPPLSL | HLA-A*02: 01 |
| 141 | WMWQPPLSI | LQWQPPLSL | HLA-A*02: 01 |
| 142 | WMWQPPLSL | LQWQPPLSL | HLA-A*02: 01 |
| 143 | WMWQPPLSV | LQWQPPLSL | HLA-A*02: 01 |
| 186 | YLWQPPLSI | LQWQPPLSL | HLA-A*02: 01 |
| 187 | YLWQPPLSL | LQWQPPLSL | HLA-A*02: 01 |
| 188 | YLWQPPLSV | LQWQPPLSL | HLA-A*02: 01 |
| 222 | YMWQPPLSI | LQWQPPLSL | HLA-A*02: 01 |
| 223 | YMWQPPLSL | LQWQPPLSL | HLA-A*02: 01 |
| 224 | YMWQPPLSV | LQWQPPLSL | HLA-A*02: 01 |
| 230 | YQWQPPLSV | LQWQPPLSL | HLA-A*02: 01 |
| 12 | FIVKPLPPV | NIVKPLPPV | HLA-A*02: 01 |
| 34 | FLVKPLPPI | NIVKPLPPV | HLA-A*02: 01 |
| 35 | FLVKPLPPL | NIVKPLPPV | HLA-A*02: 01 |
| 36 | FLVKPLPPV | NIVKPLPPV | HLA-A*02: 01 |
| 70 | FMVKPLPPI | NIVKPLPPV | HLA-A*02: 01 |
| 71 | FMVKPLPPL | NIVKPLPPV | HLA-A*02: 01 |
| 72 | FMVKPLPPV | NIVKPLPPV | HLA-A*02: 01 |
| 119 | WLVKPLPPV | NIVKPLPPV | HLA-A*02: 01 |
| 139 | WMVKPLPPV | NIVKPLPPV | HLA-A*02: 01 |
| 161 | YIVKPLPPV | NIVKPLPPV | HLA-A*02: 01 |
| 182 | YLVKPLPPI | NIVKPLPPV | HLA-A*02: 01 |
| 183 | YLVKPLPPL | NIVKPLPPV | HLA-A*02: 01 |
| 184 | YLVKPLPPV | NIVKPLPPV | HLA-A*02: 01 |
| 218 | YMVKPLPPI | NIVKPLPPV | HLA-A*02: 01 |
| 219 | YMVKPLPPL | NIVKPLPPV | HLA-A*02: 01 |
| 220 | YMVKPLPPV | NIVKPLPPV | HLA-A*02: 01 |
| 21 | FLFYWYEGI | NLFYWYEGL | HLA-A*02: 01 |
| 22 | FLFYWYEGL | NLFYWYEGL | HLA-A*02: 01 |
| 23 | FLFYWYEGV | NLFYWYEGL | HLA-A*02: 01 |
| 54 | FMFYWYEGI | NLFYWYEGL | HLA-A*02: 01 |
| 55 | FMFYWYEGL | NLFYWYEGL | HLA-A*02: 01 |
| 56 | FMFYWYEGV | NLFYWYEGL | HLA-A*02: 01 |
| 110 | WLFYWYEGV | NLFYWYEGL | HLA-A*02: 01 |
| 129 | WMFYWYEGV | NLFYWYEGL | HLA-A*02: 01 |
| 168 | YLFYWYEGI | NLFYWYEGL | HLA-A*02: 01 |
| 169 | YLFYWYEGL | NLFYWYEGL | HLA-A*02: 01 |
| 170 | YLFYWYEGV | NLFYWYEGL | HLA-A*02: 01 |
| 201 | YMFYWYEGI | NLFYWYEGL | HLA-A*02: 01 |
| 202 | YMFYWYEGL | NLFYWYEGL | HLA-A*02: 01 |
| 203 | YMFYWYEGV | NLFYWYEGL | HLA-A*02: 01 |
| 92 | FSSWAETTY | QSSWAETTY | HLA-A*01: 01 |
| 240 | YSSWAETTY | QSSWAETTY | HLA-A*01: 01 |
| 101 | RLIGSETWK | RNIGSETWK | HLA-A*03: 01 |
| 102 | RMIGSETWK | RNIGSETWK | HLA-A*03: 01 |
| 210 | YMLAIGCLY | VCLAIGCLY | HLA-A*01: 01 |
| 30 | FLNETYTLK | VENETYTLK | HLA-A*03: 01 |
| 63 | FMNETYTLK | VENETYTLK | HLA-A*03: 01 |
| 103 | VLNETYTLK | VENETYTLK | HLA-A*03: 01 |
| 104 | VMNETYTLK | VENETYTLK | HLA-A*03: 01 |
| 116 | WLNETYTLK | VENETYTLK | HLA-A*03: 01 |
| 136 | WMNETYTLK | VENETYTLK | HLA-A*03: 01 |
| 177 | YLNETYTLK | VENETYTLK | HLA-A*03: 01 |
| 211 | YMNETYTLK | VENETYTLK | HLA-A*03: 01 |
| 31 | FLPFGFILV | WLPFGFILI | HLA-A*02: 01 |
| 64 | FMPFGFILV | WLPFGFILI | HLA-A*02: 01 |
| 178 | YLPFGFILV | WLPFGFILI | HLA-A*02: 01 |
| 212 | YMPFGFILV | WLPFGFILI | HLA-A*02: 01 |
| 267 | YLPFGFILV | WLPFGFILI | HLA-A*02: 01 |
| 268 | VLPFGFILV | WLPFGFILI | HLA-A*02: 01 |
| 269 | NLPFGFILV | WLPFGFILI | HLA-A*02: 01 |
| 270 | RLPFGFILV | WLPFGFILI | HLA-A*02: 01 |
| 271 | SLPFGFILV | WLPFGFILI | HLA-A*02: 01 |
| 272 | TLPFGFILV | WLPFGFILI | HLA-A*02: 01 |
| 273 | CLPFGFILV | WLPFGFILI | HLA-A*02: 01 |
| 274 | LLPFGFILV | WLPFGFILI | HLA-A*02: 01 |
| 41 | FLYLLCSWK | WQYLLCSWK | HLA-A*03: 01 |
| 77 | FMYLLCSWK | WQYLLCSWK | HLA-A*03: 01 |
| 189 | YLYLLCSWK | WQYLLCSWK | HLA-A*03: 01 |
| 225 | YMYLLCSWK | WQYLLCSWK | HLA-A*03: 01 |
| 20 | FLDKQCWEV | WSDKQCWEG | HLA-A*01: 01 |
| 53 | FMDKQCWEV | WSDKQCWEG | HLA-A*01: 01 |
| 86 | FSDKQCWEI | WSDKQCWEG | HLA-A*01: 01 |
| 87 | FSDKQCWEL | WSDKQCWEG | HLA-A*01: 01 |
| 88 | FSDKQCWEV | WSDKQCWEG | HLA-A*01: 01 |
| 128 | WMDKQCWEV | WSDKQCWEG | HLA-A*01: 01 |
| 150 | WSDKQCWEI | WSDKQCWEG | HLA-A*01: 01 |
| 151 | WSDKQCWEL | WSDKQCWEG | HLA-A*01: 01 |
| 152 | WSDKQCWEV | WSDKQCWEG | HLA-A*01: 01 |
| 167 | YLDKQCWEV | WSDKQCWEG | HLA-A*01: 01 |
| 200 | YMDKQCWEV | WSDKQCWEG | HLA-A*01: 01 |
| 234 | YSDKQCWEI | WSDKQCWEG | HLA-A*01: 01 |
| 235 | YSDKQCWEL | WSDKQCWEG | HLA-A*01: 01 |
| 236 | YSDKQCWEV | WSDKQCWEG | HLA-A*01: 01 |
| 279 | FLPFGFILPV | WLPFGFILI(L) | HLA-A*02: 01 |

Those 255 sequence variants can be further defined based on the sequence of reference epitope in human IL13RA2. Accordingly, those 255 sequence variants may be categorized in a plurality of distinct families according to their reference epitope in IL13RA2:
Family «AIGCLYTFL» (SEQ ID NO: 243), which family includes the amino acid sequences of SEQ ID NO: 1-3, 9-11, 24-26, 57-59, 111-113, 130-132, 158-160, 171-173, 204-206;
Family «ASDYKDFYI» (SEQ ID NO: 244) which family includes the amino acid sequences of SEQ ID NO: 89-91, 153-155, 237-239;
Family «CLYTFLIST» (SEQ ID NO: 245) which family includes the amino acid sequences of SEQ ID NO: 4, 42-45, 78-81, 123, 144-146, 190-193, 226-229;
Family «CSDDGIWSE» (SEQ ID NO: 246) which family includes the amino acid sequences of SEQ ID NO: 5-7, 16-18, 49-51, 83-85, 107-109, 125-127, 147-149, 163-165, 197-198, 231-233;
Family «EASDYKDFY» (SEQ ID NO: 247) which family includes the amino acid sequences of SEQ ID NO: 8, 32, 66, 105, 117, 137, 157, 180, 214;
Family «ETWKTIITK» (SEQ ID NO: 248) which family includes the amino acid sequences of SEQ ID NO: 37, 73, 93, 140, 185, 221, 241;
Family «FLISTTFGC» (SEQ ID NO: 249) which family includes the amino acid sequences of SEQ ID NO: 27-29, 60-62, 114-115, 133-135, 174-176, 207-209;
Family «FVTGLLLRK» (SEQ ID NO: 250) which family includes the amino acid sequences of SEQ ID NO: 67, 215;
Family «GLDHALQCV» (SEQ ID NO: 251) which family includes the amino acid sequences of SEQ ID NO: 19, 52, 166, 199;
Family «ILVIFVTGL» (SEQ ID NO: 252) which family includes the amino acid sequences of SEQ ID NO: 69, 217;
Family «KVQDMCVYY» (SEQ ID NO: 253) which family includes the amino acid sequences of SEQ ID NO: 65, 94, 156, 179, 213, 242;
Family «LDTNYNLFY» (SEQ ID NO: 254) which family includes the amino acid sequences of SEQ ID NO: 33, 68, 95, 97, 118, 138, 181, 216;
Family «LLCSWKPGI» (SEQ ID NO: 255) which family includes the amino acid sequences of SEQ ID NO: 13-15, 46-48, 106, 124, 162, 194-195;
Family «LQWQPPLSL» (SEQ ID NO: 256) which family includes the amino acid sequences of SEQ ID NO: 38-40, 74-76, 82, 96-100, 120-122, 141-143, 186-188, 222-224, 230;
Family «NIVKPLPPV» (SEQ ID NO: 257) which family includes the amino acid sequences of SEQ ID NO: 12, 34-36, 70-72, 119, 139, 161, 182-184, 218-220;
Family «NLFYWYEGL» (SEQ ID NO: 258) which family includes the amino acid sequences of SEQ ID NO: 21-23, 54-56, 110, 129, 168-170, 201-203;
Family «QSSWAETTY» (SEQ ID NO: 259) which family includes the amino acid sequences of SEQ ID NO: 92, 240;
Family «RNIGSETWK» (SEQ ID NO: 260) which family includes the amino acid sequences of SEQ ID NO: 101, 102;
Family «VCLAIGCLY» (SEQ ID NO: 261) including sequence SEQ ID NO: 210;
Family «VENETYTLK» (SEQ ID NO: 262) which family includes the amino acid sequences of SEQ ID NO: 30, 63, 103-104, 116, 136, 177, 211;
Family «WLPFGFILI» (SEQ ID NO: 263) which family includes the amino acid sequences of SEQ ID NO: 31, 64, 178, 212, 267-274 and 279;
Family «WQYLLCSWK» (SEQ ID NO: 264) which family includes the amino acid sequences of SEQ ID NO: 41, 77, 189, 225;
Family «WSDKQCWEG» (SEQ ID NO: 265) which family includes the amino acid sequences of SEQ ID NO: 20, 53, 86-88, 128, 150-152, 167, 200, 234-236;
Family «WLPFGFILIL» (SEQ ID NO: 276) including sequence SEQ ID NO: 279.

Accordingly, a preferred sequence variant belongs to the Family «AIGCLYTFL» (SEQ ID NO: 243). Accordingly, a preferred sequence variant comprises or consists of an amino acid sequence as set forth in any one of SEQ ID NO: 1-3, 9-11, 24-26, 57-59, 111-113, 130-132, 158-160, 171-173, 204-206. Moreover, a preferred sequence variant belongs to the Family «ASDYKDFYI» (SEQ ID NO: 244). Accordingly, a preferred sequence variant comprises or consists of an amino acid sequence as set forth in any one of SEQ ID NO: 89-91, 153-155, 237-239. Moreover, a preferred sequence variant belongs to the Family «CLYTFLIST» (SEQ ID NO: 245). Accordingly, a preferred sequence variant comprises or consists of an amino acid sequence as set forth in any one of SEQ ID NO: 4, 42-45, 78-81, 123, 144-146, 190-193, 226-229. Moreover, a preferred sequence variant belongs to the Family «CSDDGIWSE» (SEQ ID NO: 246). Accordingly, a preferred sequence variant comprises or consists of an amino acid sequence as set forth in any one of SEQ ID NO: 5-7, 16-18, 49-51, 83-85, 107-109, 125-127, 147-149, 163-165, 197-198, 231-233. Moreover, a preferred sequence variant belongs to the Family «EASDYKDFY» (SEQ ID NO: 247). Accordingly, a preferred sequence variant comprises or consists of an amino acid sequence as set forth in any one of SEQ ID NO: 8, 32, 66, 105, 117, 137, 157, 180, 214. Moreover, a preferred sequence variant belongs to the Family «ETWKTIITK» (SEQ ID NO: 248). Accordingly, a preferred sequence variant comprises or consists of an amino acid sequence as set forth in any one of SEQ ID NO: 37, 73, 93, 140, 185, 221, 241. Moreover, a preferred sequence variant belongs to the Family «FLISTTFGC» (SEQ ID NO: 249). Accordingly, a preferred sequence variant comprises or consists of an amino acid sequence as set forth in any one of SEQ ID NO: 27-29, 60-62, 114-115, 133-135, 174-176, 207-209. Moreover, a preferred sequence variant belongs to the Family «FVTGLLLRK» (SEQ ID NO: 250). Accordingly, a preferred sequence variant comprises or consists of an amino acid sequence as set forth in any one of SEQ ID NO: 67, 215. Moreover, a preferred sequence variant belongs to the Family «GLDHALQCV» (SEQ ID NO: 251). Accordingly, a preferred sequence variant comprises or consists of an amino acid sequence as set forth in any one of SEQ ID NO: 19, 52, 166, 199. Moreover, a preferred sequence variant belongs to the Family «ILVIFVTGL» (SEQ ID NO: 252). Accordingly, a preferred sequence variant comprises or consists of an amino acid sequence as set forth in any one of SEQ ID NO: 69, 217. Moreover, a preferred sequence variant belongs to the Family «KVQDMCVYY» (SEQ ID NO: 253). Accordingly, a preferred sequence variant comprises or consists of an amino acid sequence as set forth in any one of SEQ ID NO: 65, 94, 156, 179, 213, 242. Moreover, a preferred sequence variant belongs to the Family «LDTNYNLFY» (SEQ ID NO: 254). Accordingly, a preferred sequence variant comprises or consists of an amino acid sequence as set forth in any one of SEQ ID NO: 33, 68, 95, 97, 118, 138, 181, 216. Moreover, a preferred sequence variant belongs to the Family «LLCSWKPGI» (SEQ ID NO: 255). Accordingly, a preferred sequence variant comprises or consists of an amino acid sequence as set forth in any one of SEQ ID NO: 13-15, 46-48, 106, 124, 162, 194-195. Moreover, a preferred sequence variant belongs to the Family «LQWQPPLSL» (SEQ ID NO: 256). Accordingly, a preferred sequence variant comprises or consists of an amino acid sequence as set forth in any one of SEQ ID NO: 38-40, 74-76, 82, 96-100, 120-122, 141-143, 186-188, 222-224, 230. Moreover, a preferred sequence variant belongs to the Family «NIVKPLPPV» (SEQ ID NO: 257). Accordingly, a preferred sequence variant comprises or consists of an amino acid sequence as set forth in any one of SEQ ID NO: 12, 34-36, 70-72, 119, 139, 161, 182-184, 218-220. Moreover, a preferred sequence variant belongs to the Family «NLFYWYEGL» (SEQ ID NO: 258). Accordingly, a preferred sequence variant comprises or consists of an amino acid sequence as set forth in any one of SEQ ID NO: 21-23, 54-56, 110, 129, 168-170, 201-203. Moreover, a preferred sequence variant belongs to the Family «QSSWAETTY» (SEQ ID NO: 259). Accordingly, a preferred sequence variant comprises or consists of an amino acid sequence as set forth in any one of SEQ ID NO: 92, 240. Moreover, a preferred sequence variant belongs to the Family «RNIGSETWK» (SEQ ID NO: 260). Accordingly a preferred sequence variant comprises or consists of an amino acid sequence as set forth in any one of SEQ ID NO: 101, 102. Moreover, a preferred sequence variant belongs to the Family «VCLAIGCLY» (SEQ ID NO: 261). Accordingly, a preferred sequence variant comprises or consists of an amino acid sequence as set forth in any one of SEQ ID NO: 210. Moreover, a preferred sequence variant belongs to the Family «VENETYTLK» (SEQ ID NO: 262). Accordingly, a preferred sequence variant comprises or consists of an amino acid sequence as set forth in any one of SEQ ID NO: 30, 63, 103-104, 116, 136, 177, 211. Moreover, a preferred sequence variant belongs to the Family «WLPFGFILI» (SEQ ID NO: 263). Accordingly, a preferred sequence variant comprises or consists of an amino acid sequence as set forth in any one of SEQ ID NO: 31, 64, 178, 212, 267-274 and 279. Moreover, a preferred sequence variant belongs to the Family «WQYLLCSWK» (SEQ ID NO: 264). Accordingly, a preferred sequence variant comprises or consists of an amino acid sequence as set forth in any one of SEQ ID NO: 41, 77, 189, 225. Moreover, a preferred sequence variant belongs to the Family «WSDKQCWEG» (SEQ ID NO: 265). Accordingly, a preferred sequence variant comprises or consists of an amino acid sequence as set forth in any one of SEQ ID NO: 20, 53, 86-88, 128, 150-152, 167, 200, 234-236. Moreover, a preferred sequence variant belongs to the Family «RNIGSETWK» (SEQ ID NO: 276). Accordingly, a preferred sequence variant comprises or consists of an amino acid sequence as set forth in any one of SEQ ID NO: 279.

More preferably, the (poly)peptide for use according to the present invention is selected from the group consisting of peptides or polypeptides comprising or consisting of an amino acid sequence according to any one of SEQ ID NOs: 12, 19, 21, 22, 23, 27, 28, 29, 31, 34, 35, 36, 52, 54, 55, 56, 60, 61, 62, 64, 69, 70, 71, 72, 110, 114, 115, 119, 129, 133, 134, 135, 139, 161, 166, 168, 169, 170, 174, 175, 176, 178, 182, 183, 184, 199, 201, 202, 203, 207, 208, 209, 212, 217, 218, 219, 220, 267, 268, 269 and 279.

According to an even more preferred embodiment, the (poly)peptide for use according to the present invention is selected from the group consisting of peptides or polypeptides comprising, or consisting of, any one of the amino acid sequences SEQ ID NO: 31, 64, 178, 192, 212, 267 and 279. Even more preferably, the (poly)peptide comprises or consists of an amino acid sequence according to SEQ ID NO: 31, SEQ ID NO: 192 or SEQ ID NO: 279.

Most preferably, the (poly)peptide for use according to the present invention is a peptide or polypeptide comprising, or consisting of, the amino acid sequence as set forth in SEQ ID NO: 31. According to another preferred exemplified embodiment, the (poly)peptide for use according to the present invention is a peptide or polypeptide comprising, or consisting of, the amino acid sequence of SEQ ID NO: 192. According to a further preferred exemplified embodiment, the (poly)peptide for use according to the present invention is a peptide or polypeptide comprising, or consisting of, the amino acid sequence of SEQ ID NO: 267. According to a further preferred exemplified embodiment, the (poly)peptide for use according to the present invention is a peptide or polypeptide comprising, or consisting of, the amino acid sequence of SEQ ID NO: 279.

Preferably, the (poly)peptide for use according to the present invention comprises a sequence variant of the IL13RA2 epitope, wherein the sequence variant is a microbiota sequence variant.

The term “microbiota sequence variant” refers to a sequence variant of an IL13RA2 epitope, which is found in microbiota (for example, as a part of a microbiota protein, which is distinct from IL13RA2). In other words, the “microbiota sequence variant” is a microbiota sequence (sequence of microbiota origin), which is a sequence variant of an IL13RA2 epitope. That is, the “microbiota sequence variant” is a microbiota sequence (sequence of microbiota origin) which is similar, but contains at least one alteration, in comparison to an IL13RA2 epitope. Accordingly, the “microbiota sequence variant” is a sequence naturally occurring in microbiota (and nota sequence variant of a microbiota sequence).

Accordingly, the term “microbiota sequence variant” refers to a (poly)peptide sequence found in microbiota, i.e. of microbiota origin (once the sequence was identified in microbiota, it can usually also be obtained by recombinant measures well-known in the art). A “microbiota sequence variant” may refer to a complete (poly)peptide found in microbiota or, preferably, to a fragment of a (complete) microbiota (poly)peptide/protein having a length of at least 5 amino acids, preferably at least 6 amino acids, more preferably at least 7 amino acids, and even more preferably at least 8 amino acids. For example, the “microbiota sequence variant” may be a fragment of a microbiota protein/nucleic acid molecule, the fragment having a length of 9 or 10 amino acids. Preferably, the microbiota sequence variant is a fragment of a microbiota protein as described above. Preferably, the microbiota sequence variant has a length of 8-12 amino acids, more preferably the microbiota sequence variant has a length of 8-10 amino acids. Peptides having such a length can bind to MHC (major histocompatibility complex) class I (MHC I), which is crucial for a cytotoxic T-lymphocyte (CTL) response. It is also preferred that the microbiota sequence variant has a length of 13-24 amino acids. Peptides having such a length can bind to MHC (major histocompatibility complex) class II (MHC II), which is crucial for a CD4+ T-cell (T helper cell) response.

The term “microbiota”, as used herein, refers to commensal, symbiotic and pathogenic microorganisms found in and on all multicellular organisms studied to date from plants to animals. In particular, microbiota have been found to be crucial for immunologic, hormonal and metabolic homeostasis of their host. Microbiota include bacteria, archaea, protists, fungi and viruses. Accordingly, the microbiota sequence variant is preferably selected from the group consisting of bacterial sequence variants, archaea sequence variants, protist sequence variants, fungi sequence variants and viral sequence variants. More preferably, the microbiota sequence variant is a bacterial sequence variant or an archaea sequence variant. Most preferably, the microbiota sequence variant is a bacterial sequence variant, i.e. a peptide of bacterial origin (which may exist in bacteria as a partial sequence of a larger bacterial (poly)peptide or protein or in the form of the peptide “as such”).

Anatomically, microbiota reside on or within any of a number of tissues and biofluids, including the skin, conjunctiva, mammary glands, vagina, placenta, seminal fluid, uterus, ovarian follicles, lung, saliva, oral cavity (in particular oral mucosa), and the gastrointestinal tract, in particular the gut. In the context of the present invention the microbiota sequence variant is preferably a sequence variant of microbiota of the gastrointestinal tract (microorganisms residing in the gastrointestinal tract), more preferably a sequence variant of microbiota of the gut (microorganisms residing in the gut). Accordingly, it is most preferred that the microbiota sequence variant is a gut bacterial sequence variant (i.e. a sequence variant of bacteria residing in the gut).

While microbiota can be found in and on many multicellular organisms (all multicellular organisms studied to date from plants to animals), microbiota found in and on mammals are preferred. Mammals contemplated by the present invention include for example human, primates, domesticated animals such as cattle, sheep, pigs, horses, laboratory rodents and the like. Microbiota found in and on humans are most preferred. Such microbiota are referred to herein as “mammalian microbiota” or “human microbiota” (wherein the term mammalian/human refers specifically to the localization/residence of the microbiota). Preferably, the IL13RA2 epitope is of the same species, in/on which the microbiota (of the microbiota sequence variant) reside. Preferably, the microbiota sequence variant is a human microbiota sequence variant. Accordingly, it is preferred that the IL13RA2 epitope is a human IL13RA2 epitope.

The inventive idea to use a microbiota sequence variant of an IL13RA2 epitope is based on the surprising finding that the present inventors identified such microbiota sequence variants of an IL13RA2 epitope in the microbiome of the human gut. Without being bound to any theory, this offers a possible way to bypass the repertoire restriction of human T cells due to clonal depletion of T cells recognizing self-antigens. In particular, antigens/epitopes distinct from self-antigens, but sharing sequence similarity with the self-antigen, (i) can still be recognized due to the cross-reactivity of the T-cell receptor (see, for example, Degauque et al., Cross-Reactivity of TCR Repertoire: Current Concepts, Challenges, and Implication for Allotransplantation. Frontiers in Immunology. 2016; 7:89. doi:10.3389/fimmu.2016.00089; Nelson et al., T cell receptor cross-reactivity between similar foreign and self peptides influences naive cell population size and autoimmunity. Immunity. 2015 Jan. 20; 42(1):95-107); and (ii) it is expected that such antigens/epitopes are recognized by T cell/TCR that have not been depleted during T cell education process. Accordingly, microbiota sequence variants of a human self antigen, such as IL13RA2, are able to elicit a strong immune response leading to clonal expansion of T cell harboring potential cross reactivity with self-antigens.

The human microbiome, which is composed of thousands of different bacterial species, is a large source of genetic diversity and potential antigenic components. The gut can be considered as the largest area of contact and exchange with microbiota. As a consequence, the gut is the largest immune organ in the body. Specialization and extrathymic T cell maturation in the human gut epithelium is known now for more than a decade. The gut contains a large panel of immune cells that could recognize our microbiota and which are tightly controlled by regulatory mechanisms.

According to the present invention, the large repertoire of bacterial species existing in the gut provides an incredible source of antigens (microbiota sequence variants) with potential similarities with human antigens, such as IL13RA2. These microbiota sequence variants are presented to specialized cells in a complex context, with large amount of co-signals delivered to immune cells as TLR activators. As a result, microbiota sequence variants may elicit full functional response and drive maturation of large T memory subset or some time lead to full clonal depletion or exhaustion. Identification of bacterial components sharing similarities with human antigens, such as IL13RA2, provides a new source for selection of epitopes of antigens, which (i) overcome the problem of T cell depletion and (ii) have already “primed” the immune system in the gut, thereby providing for stronger immune responses as compared to sequence variants of antigens/epitopes of other sources and artificially mutated antigens/epitopes.

Preferred microbiota sequence variants include the amino acid sequences according to SEQ ID NO: 31, SEQ ID NO: 192 or SEQ ID NO: 279. Accordingly, it is preferred that the (poly)peptide for use according to the present invention comprises or consists of an amino acid sequence according to SEQ ID NO: 31, SEQ ID NO: 192 or SEQ ID NO: 279.

More preferably, the (poly)peptide for use according to the present invention is a peptide or polypeptide comprising, or consisting of, the amino acid sequence as set forth in SEQ ID NO: 31. According to another more preferred exemplified embodiment, the (poly)peptide for use according to the present invention is a peptide or polypeptide comprising, or consisting of, the amino acid sequence of SEQ ID NO: 192. According to a further more preferred exemplified embodiment, the (poly)peptide for use according to the present invention is a peptide or polypeptide comprising, or consisting of, the amino acid sequence of SEQ ID NO: 279.

In some embodiments, the (poly)peptide for use according to the present invention does not comprise an amino acid sequence as set forth in SEQ ID NO: 282 (ALPFGFILV). Additionally or alternatively, the (poly)peptide may not comprise an amino acid sequence as set forth in SEQ ID NO: 283 (WLPFGFILV) or SEQ ID NO: 284 (ELPFGFILV).

In general, the (poly)peptide for use according to the present invention may be of any length. Preferably, the length of the (poly)peptide for use according to the present invention does not exceed 350 amino acids. For example, the maximum length of the (poly)peptide for use according to the present invention may be 300 or 250 amino acids. More preferably, the maximum length of the (poly)peptide for use according to the present invention does not exceed 200 amino acids, e.g., not more than 190, 180, 170, 160, 150, 140, 130, 120, 110, 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14 or 13 amino acids. Most preferably, the (poly)peptide consists of the epitope of IL13RA2 or the sequence variant thereof. Accordingly, the (poly)peptide may have a length of 8-12 amino acids, more preferably 8-10 amino acids and even more preferably 9 or 10 amino acids.

In particular, the (poly)peptide for use according to the present invention may not bind to IL-13 and/or not inhibit IL-13. Without being bound to any theory, the present inventors assume that the (poly)peptide for use according to the present invention exerts its effects by directing an immune response against IL13RA2 and/or IL13RA2 expressing cells. This approach differs considerably from the “normal” function of IL13RA2, which is binding and/or inhibiting interleukin 13 (IL-13).

Immunogenic Compounds, Nanoparticles, Cells, Compositions and Kits Comprising and Nucleic Acids Encoding the (Poly)Peptide Comprising an IL13RA2 Epitope or a Sequence Variant Thereof

Advantageously, the (poly)peptide for use according to the present invention may be in the form of an immunogenic compound. Accordingly, the present invention also provides an immunogenic compound comprising the (poly)peptide as defined above for use in prevention and/or treatment of fibrosis, an autoimmune disease and/or an inflammatory disease.

Preferably, the (poly)peptide as described above is linked to a carrier molecule, in particular a carrier protein, thereby forming an immunogenic compound for use according the present invention. Accordingly, the (poly)peptide as above defined is preferably linked to a carrier molecule, in particular a carrier protein, for example by a covalent or non-covalent bond.

Preferably, the immunogenic compound for use according to the present invention comprises or consists of an (poly)peptide of formula (I):

PepNt-CORE-PepCt (I),

wherein:
“PepNt” consists of a polypeptide having an amino acid length varying from 0 to 500 amino acid residues and located at the N-terminal end of the polypeptide of formula (I);
CORE consists of a (poly)peptide as defined above; and
“PepCt” consists of a polypeptide having an amino acid length varying from 0 to 500 amino acid residues and located at the C-terminal end of the polypeptide of formula (I).

Preferably, the (poly)peptide of formula (I) is a fusion peptide or fusion protein, in particular a recombinant fusion peptide or protein. The term “recombinant” means that it does not occur in nature.

For example, the immunogenic compound comprises, or consists of, an (poly)peptide of formula (I):

PepNt-CORE-PepCt (I), wherein:
“PepNt” consists of a polypeptide having an amino acid length varying from 0 to 500 amino acid residues and located at the N-terminal end of the polypeptide of formula (I);
CORE consists of a (poly)peptide comprising, or consisting of, an amino acid sequence selected from the group consisting of SEQ ID NO: 1 to 242, 267-274 and 279 (preferably SEQ ID NO: 31, 64, 178, 212, 267 and 279, more preferably SEQ ID NO: 31, SEQ ID NO: 192 or SEQ ID NO: 279, most preferably SEQ ID NO: 31 or SEQ ID NO: 279) and
“PepCt” consists of a polypeptide having an amino acid length varying from 0 to 500 amino acid residues and located at the C-terminal end of the polypeptide of formula (I).

According to one particular embodiment, the immunogenic compound comprises or consists of an (poly)peptide of formula (Ia) or (Ib):

PepNt-CORE (Ia); or

CORE-PepCt (Ib),

wherein “PepNt” and “PepCt” and CORE are as defined above.

According to some even more particular embodiments, the (poly)peptide or immunogenic compound as defined above comprises from 9 to 1000 amino acids; which includes 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 250, 300, 350, 400, 450, 500, 600, 700, 800, 900 and 1000 amino acids. According to said embodiment, the length of “PepNt” and “PepCt”, if applicable, are defined accordingly. Thus, “PepNt” and “PepCt”, as defined above, may comprise from 0 to 500 amino acid residues; which includes 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 250, 300, 350, 400, 450, and 500 amino acid residues.

The types of carrier molecules used for generating an immunogenic compound of the invention, such as the ones comprising or consisting of a peptide of formula (I) linked to a carrier molecule, are well in the general knowledge of the one skilled in the art. The function of the carrier molecule may be to provide cytokine help (or T-cell help) in order to enhance the immune response against IL13RA2.

Preferably, the (poly)peptide is linked to a carrier molecule, in particular to a carrier protein, preferably by covalent or non-covalent bond. The carrier molecule to which the (poly)peptide is optionally bound can be selected from a wide variety of known carriers. Examples of carrier molecules for vaccine purposes encompass proteins such as human or bovine serum albumin and keyhole limpet haemocyanin (KLH) and fatty acids. Other embodiments of carrier molecules to which an (poly)peptide (e.g. of formula (I)) may be covalently linked include bacterial toxins or toxoids, such as diphtheria, cholera, E. coli heat labile or tetanus toxoids, the N. meningitidis outer membrane protein (European patent application n° EP0372501), synthetic peptides (European patent applications n° EP0378881 and n° EP0427347), heat shock proteins (PCT application n° WO93/17712), Pertussis proteins (PCT application n° WO98/58668), protein D from H. influenzae (PCT application n° WO00/56360.) and toxin A or B from C. difficile (International patent application WO00/61761).

More preferably, the carrier protein or carrier peptide is a protein/peptide having immuno-adjuvant properties, such as providing stimulation of CD4+ Th1 cells as described herein. A preferred example thereof is a (non-IL13RA2) antigen that recalls immune memory or provides a non-specific help or could be a specific helper peptide, such as tetanus helper peptide, keyhole limpet hemocyanin peptide or PADRE peptide. In a preferred embodiment, the carrier protein or carrier peptide is a protein/peptide having immuno-adjuvant properties may be a HHD-DR3 carrier peptide MAKTIAYDEEARRGLERGLN (SEQ ID NO: 266). In particular, “PepNt” and/or “PepCt” may correspond to a carrier protein or carrier peptide, such as the HHD-DR3 carrier peptide MAKTIAYDEEARRGLERGLN (SEQ ID NO: 266). For example, the immunogenic compound comprises or consists of the carrier peptide of sequence SEQ ID NO: 266 linked covalently to the N-terminus of the (poly)peptide as described herein, e.g. as set forth in SEQ ID NO: 31, SEQ ID NO: 279 or SEQ ID NO: 192. Another preferred example is h-pAg T13L (sequence: TPPAYRPPNAPIL; SEQ ID NO: 280; Bhasin M, Singh H, Raghava G P (2003) MHCBN: a comprehensive database of MHC binding and non-binding peptides. Bioinformatics 19: 665-666). Further examples of preferred carrier proteins/peptides, in particular of helper peptides, include the UCP2 peptide (for example as described in WO 2013/135553 A1 or in Dosset M, Godet Y, Vauchy C, Beziaud L, Lone Y C, Sedlik C, Liard C, Levionnois E, Clerc B, Sandoval F, Daguindau E, Wain-Hobson S, Tartour E, Langlade-Demoyen P, Borg C, Adotévi O: Universal cancer peptide-based therapeutic vaccine breaks tolerance against telomerase and eradicates established tumor. Clin Cancer Res. 2012 Nov. 15; 18(22):6284-95. doi: 10.1158/1078-0432.CCR-12-0896. Epub 2012 Oct. 2) and the BIRC5 peptide (for example as described in EP2119726 A1 or in Widenmeyer M, Griesemann H, Stevanović S, Feyerabend S, Klein R, Attig S, Hennenlotter J, Wernet D, Kuprash D V, Sazykin A Y, Pascolo S, Stenzl A, Gouttefangeas C, Rammensee H G: Promiscuous survivin peptide induces robust CD4+ T-cell responses in the majority of vaccinated cancer patients. Int J Cancer. 2012 Jul. 1; 131(1):140-9. doi: 10.1002/ijc.26365. Epub 2011 Sep. 14). The most preferred helper peptide is the UCP2 peptide (amino acid sequence: KSVWSKLQSIGIRQH; SEQ ID NO: 281, for example as described in WO 2013/135553 A1 or in Dosset M, Godet Y, Vauchy C, Beziaud L, Lone Y C, Sedlik C, Liard C, Levionnois E, Clerc B, Sandoval F, Daguindau E, Wain-Hobson S, Tartour E, Langlade-Demoyen P, Borg C, Adotévi O: Universal cancer peptide-based therapeutic vaccine breaks tolerance against telomerase and eradicates established tumor. Clin Cancer Res. 2012 Nov. 15; 18(22):6284-95. doi: 10.1158/1078-0432.CCR-12-0896. Epub 2012 Oct. 2).

Accordingly, “PepNt” and/or “PepCt” may preferably correspond to such a protein/peptide having immuno-adjuvant properties, such as providing stimulation of CD4+ Th1 cells as described herein.

Moreover, the immunogenic compound preferably comprises or consists of such a protein/peptide having immuno-adjuvant properties, such as providing stimulation of CD4+ Th1 cells as described herein, linked covalently to the N-terminus of the (poly)peptide as defined herein, e.g. as set forth in SEQ ID NO: 31, SEQ ID NO: 279 or SEQ ID NO: 192.

According to one embodiment, the said (poly)peptide is covalently bound to the carrier molecule through a linker moiety.

The said restricted family of linker agents encompasses, or even consists of, the linker agents named GMBS, sulfo-GMBS, SMPB and sulfo-SMPB.

In some embodiments of an immunogenic compound as defined above, the said linker agent is selected form the group consisting of GMBS (N-[γ-maleimidobutyryl-oxy]succinimide ester), Sulfo-GMBS (N-[γ-maleimidobutyryl-oxy[sulfosuccinimide ester), SMPB (succinimidyl 4-[p-maleimidophenyl]butyrate) and Sulfo-SMPB (sulfosuccinimidyl 4-[p-maleimidophenyl]butyrate).

Methods for conjugating two proteins with a linker agent in general, and more particularly with a linker agent selected from the group consisting of GMBS, Sulfo-GMBS, SMPB and Sulfo-SMPB, are well known by the one skilled in the art. Illustratively, such protocols are disclosed in the leaflets that are made publicly available by the Pierce Company (Illinois, USA). GMBS, Sulfo-GMBS, SMPB and Sulfo-SMPB consist of heterobifunctional linker agents that contain both a N-hydroxysuccinimide (NHS) ester group and a maleimide group. Conjugation using GMBS, Sulfo-GMBS, SMPB or Sulfo-SMPB is usually performed by a two-step procedure. In a first step, the amine-containing protein is reacted with a several-fold molar excess of the linker agent at pH 7-9 to form amide bonds, followed by removal of excess non-reacted linker agent, usually by desalting or dialysis. In a second step, the sulfhydryl-containing molecule (e.g. peptide of formula (I)) is added to react with the maleimide groups already attached to the first protein at pH 6.5-7.5 to form stable thioether bonds.

Using SMPB or Sulfo-SMPB as linker agents for covalently linking peptides of formula (I) to the amine-containing carrier protein, leads to a conjugate of formula (II) below:

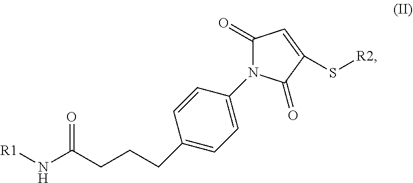

wherein:
R1 consists of one reactive group of the amine-containing carrier protein, and wherein the NH group attached thereto derives from (i) the alpha amino group located at the N-terminal end of the amine-containing carrier protein or (ii) a lateral chain amino group from a Lysine (K) amino acid residue of the amine-containing carrier protein; and
R2 consists of a (poly)peptide of formula (I), and wherein the sulphur (S) atom attached thereto derives from a sulfhydryl (SH) group of a cysteine residue located at the N-terminal end or at the C-terminal end of a (poly)peptide of formula (I). In some embodiments, the sulfhydryl moiety could be part of an unnatural amino acid, or any other molecule present at the end of the (poly)peptide of formula (I).

Using GMBS or Sulfo-GMBS as linker agents for covalently linking peptides of formula (I) to the amine-containing carrier protein, in particular the CRM197 carrier, protein leads to a conjugate of formula (III) below:

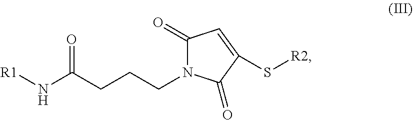

wherein:
R1 consists of one reactive group of the amine-containing carrier protein, and wherein the NH group attached thereto derives from (i) the alpha amino group located at the N-terminal end of the amine-containing carrier protein or (ii) a lateral chain amino group from a Lysine (K) amino acid residue of the amine-containing carrier protein; and
R2 consists of a (poly)peptide of formula (I), and wherein the sulphur (S) atom attached thereto derives from a sulfhydryl (SH) group of a cysteine residue located at the N-terminal end or at the C-terminal end of a (poly)peptide of formula (I). In some embodiments, the sulfhydryl moiety could be part of an unnatural amino acid, or any other molecule present at the end of the (poly)peptide of formula (I).

In a further aspect, the present invention also provides a nanoparticle loaded with
at least one (poly)peptide as defined above, or
at least one immunogenic compound as defined above;

and, optionally, with an adjuvant

for use in prevention and/or treatment of fibrosis, an autoimmune disease and/or an inflammatory disease.

Nanoparticles, in particular for use as vaccines, are known in the art and described, for example, in Shao et al., Nanoparticle-based immunotherapy for cancer, ACS Nano 2015, 9(1):16-30; Zhao et al., Nanoparticle vaccines, Vaccine 2014, 32(3):327-37; and Gregory et al., Vaccine delivery using nanoparticles, Front Cell Infect Microbiol. 2013, 3:13, doi: 10.3389/fcimb.2013.00013. eCollection 2013, Review. In particular, the nanoparticle is used for delivery of the (poly)peptide as described above (or the immunogenic compound comprising the (poly)peptide) and may optionally also act as an adjuvant. The (poly)peptide (or the immunogenic compound comprising the (poly)peptide) is typically either encapsulated within the nanoparticle or linked/bound to (decorated onto) the surface of the nanoparticle (“coating”). Compared to conventional approaches, nanoparticles can protect the payload (antigen/adjuvant) from the surrounding biological milieu, increase the half-life, minimize the systemic toxicity, promote the delivery to APCs, or even directly trigger the activation of antigen-specific T-cells. Preferably, the nanoparticle has a size (diameter) of no more than 300 nm, more preferably of no more than 200 nm and most preferably of no more than 100 nm. Such nanoparticles are adequately sheltered from phagocyte uptake, with high structural integrity in the circulation and long circulation times, capable of accumulating at target sites, and able to penetrate deep into target sites.

Preferred examples of nanoparticles include polymeric nanoparticles such as poly(ethylene glycol) (PEG) and poly (D,L-lactic-coglycolic acid) (PLGA); inorganic nanoparticles such as gold nanoparticles, iron oxide beads, iron-oxide zinc-oxide nanoparticles, carbon nanotubes and mesoporous silica nanoparticles; liposomes, such as cationic liposomes; immunostimulating complexes (ISCOM); virus-like particles (VLP); and self-assembled proteins.

Polymeric nanoparticles are nanoparticles based on/comprising polymers, such as poly(d,l-lactide-co-glycolide) (PLG), poly(d,l-lactic-coglycolic acid)(PLGA), poly(g-glutamic acid) (g-PGA), poly(ethylene glycol) (PEG), and polystyrene. Polymeric nanoparticles may entrap an antigen (e.g., the (poly)peptide or a (poly)peptide comprising the same) or bind to/conjugate to an antigen (e.g., the (poly)peptide or a (poly)peptide comprising the same). Polymeric nanoparticles may be used for delivery, e.g. to certain cells, or sustain antigen release by virtue of their slow biodegradation rate. For example, g-PGA nanoparticles may be used to encapsulate hydrophobic antigens. Polystyrene nanoparticles can conjugate to a variety of antigens as they can be surface-modified with various functional groups. Polymers, such as Poly(L-lactic acid) (PLA), PLGA, PEG, and natural polymers such as polysaccharides may also be used to synthesize hydrogel nanoparticles, which are a type of nano-sized hydrophilic three-dimensional polymer network. Nanogels have favorable properties including flexible mesh size, large surface area for multivalent conjugation, high water content, and high loading capacity for antigens. Accordingly, a preferred nanoparticle is a nanogel, such as a chitosan nanogel. Preferred polymeric nanoparticles are nanoparticles based on/comprising poly(ethylene glycol) (PEG) and poly (D,L-lactic-coglycolic acid) (PLGA).

Inorganic nanoparticles are nanoparticles based on/comprising inorganic substances, and examples of such nanoparticles include gold nanoparticles, iron oxide beads, iron-oxide zinc-oxide nanoparticles, carbon nanoparticles (e.g., carbon nanotubes) and mesoporous silica nanoparticles. Inorganic nanoparticles provide a rigid structure and controllable synthesis. For example, gold nanoparticles can be easily produced in different shapes, such as spheres, rods, cubes. Inorganic nanoparticles may be surface-modified, e.g. with carbohydrates. Carbon nanoparticles provide good biocompatibility and may be produced, for example, as nanotubes or (mesoporous) spheres. For example, multiple copies of the (poly)peptide according to the present invention (or a (poly)peptide comprising the same) may be conjugated onto carbon nanoparticles, e.g. carbon nanotubes. Mesoporous carbon nanoparticles are preferred for oral administration. Silica-based nanoparticles (SiNPs) are also preferred. SiNPs are biocompatible and show excellent properties in selective targeting and vaccine delivery. The abundant silanol groups on the surface of SiNPs may be used for further modification to introduce additional functionality, such as cell recognition, absorption of specific biomolecules, improvement of interaction with cells, and enhancement of cellular uptake. Mesoporous silica nanoparticles are particularly preferred.

Liposomes are typically formed by phospholipids, such as 1,2-dioleoyl-3-trimethylammonium propane (DOTAP). In general, cationic liposomes are preferred. Liposomes are self-assembling with a phospholipid bilayer shell and an aqueous core. Liposomes can be generated as unilameller vesicles (having a single phospholipid bilayer) or as multilameller vesicles (having several concentric phospholipid shells separated by layers of water). Accordingly, antigens can be encapsulated in the core or between different layers/shells. Preferred liposome systems are those approved for human use, such as Inflexal® V and Epaxal®.

Immunostimulating complexes (ISCOM) are cage like particles of about 40 nm (diameter), which are colloidal saponin containing micelles, for example made of the saponin adjuvant Quil A, cholesterol, phospholipids, and the (poly)peptide antigen (such as the (poly)peptide or a polypeptide comprising the same). These spherical particles can trap the antigen by apolar interactions. Two types of ISCOMs have been described, both of which consist of cholesterol, phospholipid (typically either phosphatidylethanolamine or phos-phatidylcholine) and saponin (such as QuilA).

Virus-like particles (VLP) are self-assembling nanoparticles formed by self-assembly of biocompatible capsid proteins. Due to the naturally-optimized nanoparticle size and repetitive structural order VLPs can induce potent immune responses. VLPs can be derived from a variety of viruses with sizes ranging from 20 nm to 800 nm, typically in the range of 20-150 nm. VLPs can be engineered to express additional peptides or proteins either by fusing these peptides/proteins to the particle or by expressing multiple antigens. Moreover, antigens can be chemically coupled onto the viral surface to produce bioconjugate VLPs.

Examples of self-assembled proteins include ferritin and major vault protein (MVP). Ferritin is a protein that can self-assemble into nearly-spherical 10 nm structure. Ninety-six units of MVP can self-assemble into a barrel-shaped vault nanoparticle, with a size of approximately 40 nm wide and 70 nm long. Antigens that are genetically fused with a minimal interaction domain can be packaged inside vault nanoparticles by self-assembling process when mixed with MVPs. Accordingly, the antigen (such as the (poly)peptide according to the present invention of a polypeptide comprising the same) may be fused to a self-assembling protein or to a fragment/domain thereof, such as the minimal interaction domain of MVP. Accordingly, the present invention also provides a fusion protein comprising a self-assembling protein (or a fragment/domain thereof) and the (poly)peptide according to the present invention.

In general, preferred examples of nanoparticles (NPs) include iron oxide beads, polystyrene microspheres, poly(γ-glutamic acid) (γ-PGA) NPs, iron oxide-zinc oxide NPs, cationized gelatin NPs, pluronic-stabilized poly(propylene sulfide) (PPS) NPs, PLGA NPs, (cationic) liposomes, (pH-responsive) polymeric micelles, PLGA, cancer cell membrane coated PLGA, lipid-calcium-phosphate (LCP) NPs, liposome-protamine-hyaluronic acid (LPH) NPs, polystyrene latex beads, magnetic beads, iron-dextran particles and quantum dot nanocrystals.

Preferably, the nanoparticle further comprises an adjuvant, for example a toll-like receptor (TLR) agonist. Thereby, the (poly)peptide (or the immunogenic compound comprising the (poly)peptide) can be delivered together with an adjuvant, for example to antigen-presenting cells (APCs), such as dendritic cells (DCs). The adjuvant may be encapsulated by the nanoparticle or bound to/conjugated to the surface of the nanoparticle, preferably similarly to the (poly)peptide.

Particularly preferred adjuvants are polyinosinic:polycytidylic acid (also referred to as “poly I:C”) and/or its derivative poly-ICLC. Poly I:C is a mismatched double-stranded RNA with one strand being a polymer of inosinic acid, the other a polymer of cytidylic acid. Poly I:C is an immunostimulant known to interact with toll-like receptor 3 (TLR3). Poly I:C is structurally similar to double-stranded RNA, which is the “natural” stimulant of TLR3. Accordingly, poly I:C may be considered a synthetic analog of double-stranded RNA. Poly-ICLC is a synthetic complex of carboxymethylcellulose, polyinosinic-polycytidylic acid, and poly-L-lysine double-stranded RNA. Similar to poly I:C, also poly-ICLC is a ligand for TLR3. Poly 1:C and poly-ICLC typically stimulate the release of cytotoxic cytokines. A preferred example of poly-ICLC is Hiltonol®.

In a further aspect, the present invention also provides a cell loaded with the (poly)peptide as described above or the immunogenic compound as described above for use in prevention and/or treatment of fibrosis, an autoimmune disease and/or an inflammatory disease.

A preferred cell is an antigen presenting cell (APC), more preferably a dendritic cell (DC).

Antigen-presenting cells (APCs) are of particular interest, as their main function is to process antigens and present it on the cell surface to the T cells of the immune system, so as to initiate and modulate T-cell responses in vivo. In the context of the present invention, it is preferred that the APCs are loaded with the (poly)peptide(s) and/or immunogenic compound(s) according to the invention, which can be done by exposing APCs in vitro with said (poly)peptide(s) and/or immunogenic compound(s) (Rizzo M M, Alaniz L, Mazzolini G. Ex vivo loading of autologous dendritic cells with tumor antigens. Methods Mol Biol. 2014; 1139:41-4; Rolinski J, Hus I. Breaking immunotolerance of tumors: a new perspective for dendritic cell therapy. J Immunotoxicol. 2014 October; 11(4):311-8).

Preferred antigen-presenting cells according to the invention are dendritic cells (DCs). It can indeed be advantageous to combine at least one (poly)peptide or immunogenic compound according to the invention with dendritic cells, as those are the most potent antigen-presenting cells and have been reported to be frequently functionally defective in cancer patients. Dendritic cells can be easily obtained by the skilled person in the art from either healthy compatible donors (i.e. the dendritic cells are HLA-related) or from the patient himself provided that they are functional (i.e. the dendritic cells are autologous), for example by direct isolation from the peripheral blood, or by derivation from peripheral blood cells such as CD14+ monocytes or CD34+ hematopoietic precursors (Figdor C G, de Vries I J, Lesterhuis W J, Melief C J. Dendritic cell immunotherapy: mapping the way. Nat Med. 2004 May; 10(5):475-80). Dendritic cells can indeed be distinguished from other cells of peripheral blood by their surface markers, such as S100, p55, CD83, and/or OX62, and may thus be isolated and purified based on said markers using cell cultures techniques well-known in the art.

In a further aspect, the present invention also provides a nucleic acid for use in prevention and/or treatment of fibrosis, an autoimmune disease and/or an inflammatory disease, the nucleic acid encoding an (poly)peptide for use according to the present invention or an immunogenic compound for use according to the present invention, wherein the immunogenic compound may be a (poly)peptide of formula (I) as described above.

Nucleic acids preferably comprise single stranded, double stranded or partially double stranded nucleic acids, preferably selected from genomic DNA, cDNA, RNA, siRNA, antisense DNA, antisense RNA, ribozyme, complimentary RNA/DNA sequences with or without expression elements, a mini-gene, gene fragments, regulatory elements, promoters, and combinations thereof. Further preferred examples of nucleic acid (molecules) and/or polynucleotides include, e.g., a recombinant polynucleotide, a vector, an oligonucleotide, an RNA molecule such as an rRNA, an mRNA or a tRNA, or a DNA molecule as described above. It is thus preferred that the nucleic acid (molecule) is a DNA molecule or an RNA molecule; preferably selected from genomic DNA; cDNA; rRNA; mRNA; antisense DNA; antisense RNA; complimentary RNA and/or DNA sequences; RNA and/or DNA sequences with or without expression elements, regulatory elements, and/or promoters; a vector; and combinations thereof.

Accordingly, the nucleic acid molecule may be a vector. The term “vector”, as used in the context of the present invention, refers to a nucleic acid molecule, preferably to an artificial nucleic acid molecule, i.e. a nucleic acid molecule which does not occur in nature. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a desired (poly)peptide according to the present invention. An expression vector may be used for production of expression products such as RNA, e.g. mRNA, or peptides, polypeptides or proteins. For example, an expression vector may comprise sequences needed for transcription of a sequence stretch of the vector, such as a promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. Preferably, a vector is a DNA molecule. For example, a vector in the sense of the present application comprises a cloning site, a selection marker, such as an antibiotic resistance factor, and a sequence suitable for multiplication of the vector, such as an origin of replication. Preferably, a vector in the context of the present application is a plasmid vector. Preferably, a vector in the context of the present application is an expression vector. A preferred vector is a vector for expression in bacterial cells. More preferably, the vector is useful for expression in so-called “live bacterial vaccine vectors”, wherein live bacterial cells (such as bacteria or bacterial spores, e.g., endospores, exospores or microbial cysts) can serve as vaccines. Preferred examples thereof are described in da Silva et al., Live bacterial vaccine vectors: an overview; Braz J Microbiol. 2015 Mar. 4; 45(4):1117-29.

Nucleic acids encoding (poly)peptides according to the invention may be in the form of naked nucleic acids, or nucleic acids cloned into plasmids or viral vectors (Tregoning and Kinnear, Using Plasmids as DNA Vaccines for Infectious Diseases. Microbiol Spectr. 2014 December; 2(6). doi: 10.1128/microbiolspec.PLAS-0028-2014), the latter being particularly preferred. Examples of suitable viral vectors according to the invention include, without limitation, retrovirus, adenovirus, adeno-associated virus (AAV), herpes virus and poxvirus vectors. It is within the skill of the person in the art to clone a nucleic acid into a plasmid or viral vector, using standard recombinant techniques in the art.

Preferably, the nucleic acid is a DNA molecule or an RNA molecule; preferably selected from genomic DNA; cDNA; siRNA; rRNA; mRNA; antisense DNA; antisense RNA; ribozyme; complimentary RNA and/or DNA sequences; RNA and/or DNA sequences with or without expression elements, regulatory elements, and/or promoters; a vector; and combinations thereof.

In a further aspect, the present invention also provides a host cell for use in prevention and/or treatment of fibrosis, an autoimmune disease and/or an inflammatory disease, the host cell comprising the nucleic acid for use according to the present invention, wherein the nucleic acid is preferably a vector. Preferably, the host cell is a bacterial cell. Such a host cell may be preferably used for production of the (poly)peptide according to the present invention or the immunogenic compound according to the present invention. Moreover, such a host cell may also be an active component in a vaccine.

Preferably, the host cell is a bacterial cell, more preferably a gut bacterial cell. Such a bacterial host cell may serve as “live bacterial vaccine vector”, wherein live bacterial cells (such as bacteria or bacterial spores, e.g., endospores, exospores or microbial cysts) can serve as vaccines. Preferred examples thereof are described in da Silva et al., Live bacterial vaccine vectors: an overview; Braz J Microbiol. 2015 Mar. 4; 45(4):1117-29.

Bacterial cells (such as bacteria or bacterial spores, e.g., endospores, exospores or microbial cysts), in particular (entire) gut bacterial species, can be advantageous, as they have the potential to trigger a greater immune response than the (poly)peptides or nucleic acids they contain.

Alternatively, bacterial cells, in particular gut bacteria, according to the invention may be in the form of probiotics, i.e. of live gut bacterium, which can thus be used as food additive due to the health benefits it can provide. Those can be for example lyophilized in granules, pills or capsules, or directly mixed with dairy products for consumption.

In a further aspect, the present invention provides a pharmaceutical composition comprising
the (poly)peptide as described above;
the immunogenic compound as described above;
the nanoparticle as described above;
the cell as defined as described above;
the nucleic acid as described above; or the host cell as described above

for use in prevention and/or treatment of fibrosis, an autoimmune disease and/or an inflammatory disease.

Preferably, the pharmaceutical composition further comprises and, optionally, one or more pharmaceutically acceptable excipients or carriers. Preferably, the pharmaceutical composition is an immunogenic composition.

The pharmaceutical composition for use according to the invention may be in any form suitable for the purposes of the invention. For example, said composition may be in a form suitable for parenteral, enteral or topical administration, such as a liquid suspension, a solid dosage form (granules, pills, capsules or tablets), or a paste or gel. It is within the skill of the person in the art to select the appropriate form of the composition for the intended purpose.

Indeed, in the context of the present invention, it can be particularly advantageous to use (poly)peptides, or nucleic acids encoding the same, because of their ease of manufacturing at a low cost and relative safety with no potential for reassortment, infection or recombination.

(Poly)peptides for use according to the invention may be administered in the form of immunogenic compounds for use according to the present invention, cells loaded therewith for use according to the present invention, nanoparticles for use according to the present invention, nucleic acids for use according to the present invention, host cells for use according to the present invention and/or pharmaceutical compositions for use according to the present invention as described herein.

According to one embodiment, they may be administered in the form of a micro-organism such as a gut bacterial species. Entire gut bacterial species can also be advantageous as they have the potential to trigger a greater immune response than the (poly)peptides or nucleic acids they contain. Alternatively, gut bacteria according to the invention may be in the form of probiotics, i.e. of live gut bacterium, which can thus be used as food additive thanks to the health benefits it can provide. Those can be for example lyophilized in granules, pills or capsules, or directly mixed with dairy products for consumption.

In the context of the present invention co-administration of several (poly)peptides for use according to the invention is particularly preferred, so as to enhance the immune response. Thus, according to a preferred embodiment, the composition of the invention comprises at least 2 (poly)peptides (which may be in the form of immunogenic compounds) as defined above, such as at least 3 (poly)peptides, or at least 4 (poly)peptides, or at least 5 (poly)peptides, or at least 6 (poly)peptides, or at least 7 (poly)peptides, or at least 8 (poly)peptides, or at least 9 (poly)peptides, or at least 10 (poly)peptides, or at least 11 (poly)peptides, or at least 12 (poly)peptides, or at least 13 (poly)peptides, or at least 14 (poly)peptides, or at least 15 (poly)peptides, or at least 20 (poly)peptides, or at least 25 (poly)peptides, or at least 50 (poly)peptides, or at least 100 (poly)peptides, or at least 500 (poly)peptides, or at least 1000 (poly)peptides, or at least 1500 (poly)peptides.

For example, it is also preferred that the pharmaceutical composition for use according to the present invention comprises
at least two distinct (poly)peptides for use according to the present invention;
at least two distinct immunogenic compounds for use according to the present invention, wherein each of the at least two immunogenic compounds comprises a distinct (poly)peptide for use according to the present invention;
at least two distinct nanoparticles for use according to the present invention, wherein each of the at least two nanoparticles is loaded with a distinct (poly)peptide for use according to the present invention; or
at least two distinct nucleic for use according to the present invention, wherein each of the at least two nucleic acids encodes a distinct (poly)peptide for use according to the present invention.

In a particularly preferred embodiment the (poly)peptide comprising or consisting of SEQ ID NO: 31 is combined with the (poly)peptide comprising or consisting of SEQ ID NO: 192. In other words, the composition according to the present invention preferably comprises
(i) an immunogenic compound comprising an (poly)peptide having an amino acid sequence as set forth in SEQ ID NO: 31 and an immunogenic compound comprising an (poly)peptide having an amino acid sequence as set forth in SEQ ID NO: 192;
(ii) a (poly)peptide having an amino acid sequence as set forth in SEQ ID NO: 31 and a (poly)peptide having an amino acid sequence as set forth in SEQ ID NO: 192;
(iii) a nanoparticle loaded with an (poly)peptide having an amino acid sequence as set forth in SEQ ID NO: 31 and a nanoparticle loaded with an (poly)peptide having an amino acid sequence as set forth in SEQ ID NO: 192; or
(iv) a nucleic acid comprising a polynucleotide encoding an (poly)peptide having an amino acid sequence as set forth in SEQ ID NO: 31 and a nucleic acid comprising a polynucleotide encoding an (poly)peptide having an amino acid sequence as set forth in SEQ ID NO: 192.

It is also particularly preferred that the (poly)peptide comprising or consisting of SEQ ID NO: 279 is combined with the (poly)peptide comprising or consisting of SEQ ID NO: 192. In other words, the composition according to the present invention preferably comprises
(i) an immunogenic compound comprising an (poly)peptide having an amino acid sequence as set forth in SEQ ID NO: 279 and an immunogenic compound comprising an (poly)peptide having an amino acid sequence as set forth in SEQ ID NO: 192;
(ii) a (poly)peptide having an amino acid sequence as set forth in SEQ ID NO: 279 and a (poly)peptide having an amino acid sequence as set forth in SEQ ID NO: 192;
(iii) a nanoparticle loaded with an (poly)peptide having an amino acid sequence as set forth in SEQ ID NO: 279 and a nanoparticle loaded with an (poly)peptide having an amino acid sequence as set forth in SEQ ID NO: 192; or
(iv) a nucleic acid comprising a polynucleotide encoding an (poly)peptide having an amino acid sequence as set forth in SEQ ID NO: 279 and a nucleic acid comprising a polynucleotide encoding an (poly)peptide having an amino acid sequence as set forth in SEQ ID NO: 192.

The composition according to the invention can further comprise other active agents, for example such, which can enhance the effects of the (poly)peptide or immunogenic compound. Alternatively, the composition may not comprise any other active agents (i.e., other than the (poly)peptide according to the present invention, the immunogenic compound according to the present invention, the nanoparticle according to the present invention, the cell according to the present invention, the nucleic acid according to the present invention, or the host cell according to the present invention).

According to a preferred embodiment, the pharmaceutical composition for use according to the present invention further comprises at least one immunostimulatory agent, in particular so as to potentiate the immune response mediated by the (poly)peptide. Preferred immunostimulatory agents according to the invention include, without limitation, immune adjuvants, antigen-presenting cells, and combinations thereof. Accordingly, it is preferred that the immunostimulatory agent is an immuno-adjuvant (immune adjuvant) or an antigen-presenting cell (APC).

Some immune adjuvants are indeed capable of favoring and prolonging the duration of interaction between an antigen and the immune system, while others are capable of recruiting and activating cells of the natural immunity so as to induce an adaptive response. The adjuvants belonging to the former category include, without limitation, mineral compounds such as alum, aluminum hydroxide, aluminum phosphate, calcium phosphate hydroxide; and oil-based emulsions such as paraffin oil, starch oil, Freund's complete/incomplete adjuvant (FCA/FIA), saponins (e.g. from the plants Quillaja, Soybean, Polygala senega). The adjuvants of belonging to the latter category include, without limitation, immunostimulatory complexes (ISCOMs) such as cytokines (e.g. GM-CSF; Interleukins such as IL-1, IL-2, IL6, RA or IL12; Tumor necrosis factors (TNFs) such as TNFα or TNFβ; Interferons IFNS such as IFNα, IFNβ, IFNγ or IFNδ, etc); ligands of toll-like receptors (TLRs) such as imiquimod, resiquimod or MPL; exosomes such as exosomes derived from dendritic cells (DCs); bacterial products such as heat-shock proteins (HSPs such as gp96, hsp90, hsp70, calreticulin, hsp110, hsp170), pathogen-associated molecular patterns (PAMPs), trehalose dimicolate (TDM), muramyldipeptide (MDP), polysaccharide (PLS) such as polysaccharide-K.

More preferably, the immune adjuvant is a protein/peptide having immuno-adjuvant properties, such as providing stimulation of CD4+ Th1 cells, as described herein. A preferred example thereof is an antigen distinct from IL13RA2 that recalls immune memory or provides a non-specific help or could be a specific helper peptide, such as tetanus helper peptide, keyhole limpet hemocyanin peptide or PADRE peptide. In particular, the immune adjuvant may be the HHD-DR3 peptide of sequence MAKTIAYDEEARRGLERGLN (SEQ ID NO: 266). This peptide represents another example of a helper peptide (having immuno-adjuvant properties), which is preferred in the context of the present invention. Another preferred example is h-pAg T13L (sequence: TPPAYRPPNAPIL; SEQ ID NO: 280; Bhasin M, Singh H, Raghava G P (2003) MHCBN: a comprehensive database of MHC binding and non-binding peptides. Bioinformatics 19: 665-666). Further examples of preferred immune adjuvants, in particular of helper peptides, include the UCP2 peptide (for example as described in WO 2013/135553 A1 or in Dosset M, Godet Y, Vauchy C, Beziaud L, Lone Y C, Sedlik C, Liard C, Levionnois E, Clerc B, Sandoval F, Daguindau E, Wain-Hobson S, Tartour E, Langlade-Demoyen P, Borg C, Adotévi O: Universal cancer peptide-based therapeutic vaccine breaks tolerance against telomerase and eradicates established tumor. Clin Cancer Res. 2012 Nov. 15; 18(22):6284-95. doi: 10.1158/1078-0432.CCR-12-0896. Epub 2012 Oct. 2) and the BIRC5 peptide (for example as described in EP2119726 A1 or in Widenmeyer M, Griesemann H, Stevanović S, Feyerabend S, Klein R, Attig S, Hennenlotter J, Wernet D, Kuprash D V, Sazykin A Y, Pascolo S, Stenzl A, Gouttefangeas C, Rammensee H G: Promiscuous survivin peptide induces robust CD4+ T-cell responses in the majority of vaccinated cancer patients. Int J Cancer. 2012 Jul. 1; 131(1):140-9. doi: 10.1002/ijc.26365. Epub 2011 Sep. 14). The most preferred helper peptide is the UCP2 peptide (amino acid sequence: KSVWSKLQSIGIRQH; SEQ ID NO: 281, for example as described in WO 2013/135553 A1 or in Dosset M, Godet Y, Vauchy C, Beziaud L, Lone Y C, Sedlik C, Liard C, Levionnois E, Clerc B, Sandoval F, Daguindau E, Wain-Hobson S, Tartour E, Langlade-Demoyen P, Borg C, Adotévi O: Universal cancer peptide-based therapeutic vaccine breaks tolerance against telomerase and eradicates established tumor. Clin Cancer Res. 2012 Nov. 15; 18(22):6284-95. doi: 10.1158/1078-0432.CCR-12-0896. Epub 2012 Oct. 2).

Particularly preferred adjuvants are polyinosinic:polycytidylic acid (also referred to as “poly I:C”) and/or its derivative poly-ICLC. Poly I:C is a mismatched double-stranded RNA with one strand being a polymer of inosinic acid, the other a polymer of cytidylic acid. Poly I:C is an immunostimulant known to interact with toll-like receptor 3 (TLR3). Poly I:C is structurally similar to double-stranded RNA, which is the “natural” stimulant of TLR3. Accordingly, poly I:C may be considered a synthetic analog of double-stranded RNA. Poly-ICLC is a synthetic complex of carboxymethylcellulose, polyinosinic-polycytidylic acid, and poly-L-lysine double-stranded RNA. Similar to poly I:C, also poly-ICLC is a ligand for TLR3. Poly I:C and poly-ICLC typically stimulate the release of cytotoxic cytokines. A preferred example of poly-ICLC is Hiltonol®.

Most preferably, the adjuvant is Montanide, such as Montanide ISA 51 VG and/or Montanide ISA 720 VG. Those adjuvants are rendering stable water-in-oil emulsions when mixed with water based antigenic media. Montanide ISA 51 VG is based on a blend of mannide monooleate surfactant and mineral oil, whereas Montanide ISA 720 VG uses a non-mineral oil (Aucouturier J, Dupuis L, Deville S, Ascarateil S, Ganne V. Montanide ISA 720 and 51: a new generation of water in oil emulsions as adjuvants for human vaccines. Expert Rev Vaccines. 2002 June; 1(1):111-8; Ascarateil S, Puget A, Koziol M-E. Safety data of Montanide ISA 51 VG and Montanide ISA 720 VG, two adjuvants dedicated to human therapeutic vaccines. Journal for Immunotherapy of Cancer. 2015; 3(Suppl 2):P428. doi:10.1186/2051-1426-3-S2-P428).

Antigen-presenting cells (APCs) are also of particular interest, as their main function is to process antigens and present it on the cell surface to the T cells of the immune system, so as to initiate and modulate T-cell responses in vivo. In the present composition, it is preferred that the APCs are loaded with the (poly)peptide(s) and/or immunogenic compound(s) according to the invention, which can be done by exposing APCs in vitro with said (poly)peptide(s) and/or immunogenic compound(s) (Rizzo et al., Ex vivo loading of autologous dendritic cells with tumor antigens. Methods Mol Biol. 2014; 1139:41-4; Rolinski and Hus, Breaking immunotolerance of tumors: a new perspective for dendritic cell therapy. J Immunotoxicol. 2014 October; 11(4):311-8).

Preferred antigen-presenting cells according to the invention are dendritic cells (DCs). It can indeed be advantageous to combine at least one (poly)peptide or immunogenic compound according to the invention with dendritic cells, as those are the most potent antigen-presenting cells and have been reported to be frequently functionally defective in cancer patients. Dendritic cells can be easily obtained by the skilled person in the art from either healthy compatible donors (i.e. the dendritic cells are HLA-related) or from the patient himself provided that they are functional (i.e. the dendritic cells are autologous), for example by direct isolation from the peripheral blood, or by derivation from peripheral blood cells such as CD14+ monocytes or CD34+ hematopoietic precursors (Emens et al., 2008). Dendritic cells can indeed be distinguished from other cells of peripheral blood by their surface markers, such as S100, p55, CD83, and/or OX62, and may thus be isolated and purified based on said markers using cell cultures techniques well-known in the art.

According to a preferred embodiment, the pharmaceutical composition may further comprise at least one anti-fibrotic, anti-inflammatory or anti-allergic agent. It is within the skill of ordinary person in the art to select the appropriate anti-fibrotic, anti-inflammatory or anti-allergic agent for the purposes of the invention. The anti-fibrotic, anti-inflammatory or anti-allergic agent can also be administered in association with the composition of the invention, either simultaneously, separately, or sequentially. Should the composition and the anti-fibrotic, anti-inflammatory or anti-allergic agent be administered in a separate or sequential manner, those may be administered in distinct pharmaceutical forms.

Thus, in another aspect, the invention relates to the combination of a composition of the invention and at least one anti-fibrotic, anti-inflammatory or anti-allergic agent for a simultaneous, separate, or sequential administration. In other terms, the invention proposes a combined use of the composition the invention and least one anti-fibrotic, anti-inflammatory or anti-allergic agent for a simultaneous, separate, or sequential administration.

Anti-fibrotic agents for combination with the (poly)peptide for use according to the present invention include TNFalpha blocking agents, HMG-CoA reductase inhibitors, angiotensin-blocking agents, Matrix-metalloproteases, Inhibitors of Tissue inhibitor of matrix metalloproteases (TIMP), Vascular endothelial growth factor (VEGF) blockade (BIBF 1120) and agents modulating TGF-β pathways, such as Metelimumab (CAT-192), GC1008, αν136 inhibitor, ALK5 inhibitors, Hepatic growth factor (HGF), Recombinant bone-morphogenic protein-7 (BMP-7), Decorin and Tyrosine-kinase inhibitors, such as Imatinib, Dasatinib, and Nolitinib. Preferred examples of anti-fibrotic agents for combination with the (poly)peptide for use according to the present invention include pirfenidone, N-acetylcysteine (NAC), etanercept, bosentan, sildenafil, and nintedanib.

Preferred examples of anti-inflammatory and anti-allergic agents for combination with the (poly)peptide for use according to the present invention include acetaminophen (paracetamol) and nonsteroidal anti-inflammatory drugs (NSAIDs), such as:
salicylates, such as acetylsalicylic acid (aspirin), Diflunisal (Dolobid), Salsalate (Disalcid), salicylic acid and other salicylates;
propionic acid derivatives, such as ibuprofen, Dexibuprofen, Naproxen, Fenoprofen, Ketoprofen, Dexketoprofen, Flurbiprofen, Oxaprozin, and Loxoprofen;
acetic acid derivatives, such as Indomethacin, Tolmetin, Sulindac, Etodolac, Ketorolac, Diclofenac, Aceclofenac, and Nabumetone;
enolic acid (Oxicam) derivatives, such as Piroxicam, Meloxicam, Tenoxicam, Droxicam, Lornoxicam, and Phenylbutazone (Bute);
anthranilic acid derivatives (Fenamates), such as Mefenamic acid, Meclofenamic acid, Flufenamic acid, and Tolfenamic acid;
selective COX-2 inhibitors (Coxibs), such as Celecoxib, Parecoxib, Etoricoxib and Firocoxib;
sulfonanilides, such as nimesulide; and
others, such as clonixin, licofelone and H-harpagide.

Aspirin, ibuprofen, naproxen, and paracetamol are most preferred.

In particular, the pharmaceutical composition for use according to the present invention may be used as a vaccine for immunotherapy. Moreover,
the immunogenic compound for use according to the present invention,
the (poly)peptide for use according to the present invention,
the nanoparticle for use according to the present invention,
the cell for use according to the present invention,
the nucleic acid for use according to the present invention,
the host cell for use according to the present invention, or
the pharmaceutical composition for use according to the present invention
   may be used as vaccine, in particular for immunotherapy for preventing and/or treating fibrosis, an autoimmune disease and/or an inflammatory disease.

As used in the context of the present invention, the term “vaccine” refers to a biological preparation that provides innate and/or adaptive immunity, typically to a particular disease, preferably fibrosis, an autoimmune disease and/or an inflammatory disease. Thus, a vaccine supports in particular an innate and/or an adaptive immune response of the immune system of a subject to be treated. For example, the (poly)peptide according to the present invention typically leads to or supports an adaptive immune response in the patient to be treated.

In the context of the present invention, the vaccine (composition) can induce a specific immune response against an antigen/protein, and is thus preferably used to prevent or treat fibrosis, an autoimmune disease and/or an inflammatory disease.

In a further aspect, the present invention also provides a kit of parts comprising at least one of
the (poly)peptide as described above;
the immunogenic compound as described above;
the nanoparticle as described above;
the cell as described above;
the nucleic acid as described above;
the host cell as described above; and/or
the pharmaceutical composition as described above
   for use in prevention and/or treatment of fibrosis, an autoimmune disease and/or an inflammatory disease.

In particular, the kit-of-parts of the invention may comprise more than one of the above described components. For example, the kit-of-parts according to the present invention may comprise at least two different immunogenic compounds, at least two different (poly)peptides, at least two different nanoparticles, at least two different cells, at least two different nucleic acids, at least two different host cells, and/or at least two different pharmaceutical compositions. Preferably, such different components comprised by the kit-of-parts as described above differ in the (poly)peptides according to the present invention, for example one component relating to a first (poly)peptide, such as an (poly)peptide having an amino acid sequence as set forth in SEQ ID NO: 31 or 279, and one component relating to a second (poly)peptide (distinct from the first (poly)peptide), such as an (poly)peptide having an amino acid sequence as set forth in SEQ ID NO: 192.

For example, the kit may comprise
a (poly)peptide as described above comprising an amino acid sequence as set forth in SEQ ID NO: 31 or 279, and
a (poly)peptide as described above comprising an amino acid sequence as set forth in SEQ ID NO: 192.

For example, the kit may comprise
an immunogenic compound as described above comprising a (poly)peptide comprising an amino acid sequence as set forth in SEQ ID NO: 31 or 279, and
an immunogenic compound as described above comprising a (poly)peptide comprising an amino acid sequence as set forth in SEQ ID NO: 192.

For example, the kit may comprise
a nanoparticle as described above comprising a (poly)peptide comprising an amino acid sequence as set forth in SEQ ID NO: 31 or 279, and
a nanoparticle as described above comprising a (poly)peptide comprising an amino acid sequence as set forth in SEQ ID NO: 192.

For example, the kit may comprise
a nucleic acid as described above encoding a (poly)peptide comprising an amino acid sequence as set forth in SEQ ID NO: 31 or 279, and
a nucleic acid as described above encoding a (poly)peptide comprising an amino acid sequence as set forth in SEQ ID NO: 192.

The various components of the kit-of-parts may be packaged in one or more containers. The above components may be provided in a lyophilized or dry form or dissolved in a suitable buffer. The kit may also comprise additional reagents including, for instance, preservatives, growth media, and/or buffers for storage and/or reconstitution of the above-referenced components, washing solutions, and the like.

In addition, the kit-of-parts according to the present invention may optionally contain instructions of use. Accordingly, it is preferred that the kit further comprises a package insert or instruction leaflet with directions to prevent and/or to treat fibrosis, an autoimmune disease and/or an inflammatory disease by using the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention.

Moreover, the present invention also provides a vaccination kit for treating, preventing and/or stabilizing fibrosis, an autoimmune disease and/or an inflammatory disease, comprising the pharmaceutical composition as described herein or the vaccine as described herein and instructions for use of said pharmaceutical composition or of said vaccine in the prevention and/or treatment of fibrosis, an autoimmune disease and/or an inflammatory disease.

It is also preferred that, in addition to any of components as described above, the kit comprises an anti-fibrotic agent, an anti-inflammatory agent or an anti-allergic agent as described herein.

Medical Treatment and Uses

As outlined above, the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention is used for the prevention and or treatment of fibrosis, an autoimmune disease and/or an inflammatory disease. Accordingly, the present invention also provides a method for ameliorating, reducing, preventing and/or treating fibrosis, an autoimmune disease and/or an inflammatory disease in a subject comprising administering to the subject
the (poly)peptide as described herein;
the immunogenic compound as described herein;
the nanoparticle as described herein;
the cell as described herein;
the nucleic acid as described herein;
the host cell as described herein;
the pharmaceutical composition as described herein; and/or
the combination as described herein.

In other words, the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention are used as medication/medicament for the prevention and or treatment of fibrosis, an autoimmune disease and/or an inflammatory disease. Accordingly, the term “medicament” or “medication” as used in the following refers to the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention.

In particular, the medicament, i.e. the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention, is used for triggering a specific immune response towards a particular antigen/protein, namely IL13RA2, so as to prevent or treat fibrosis, an autoimmune disease and/or an inflammatory disease in a patient in need thereof.

Methods of administration of a medicament are well-known to the skilled person in the art. For example the medicament can be directly administered into the subject, into the affected organ (i.e. local administration) or systemically (i.e. enteral or parenteral administration), or even applied ex vivo to cells derived from the subject or a human cell line which are subsequently administered to the subject, or even used in vitro to select a subpopulation of immune cells derived from the subject, which are then re-administered to the said subject. Administration may be by enteral or parenteral routes. Enteral administrations as used herein includes oral and rectal administrations, as well as administrations via gastric feeding tubes, duodenal feeding tubes or gastrostomy, while parenteral administrations includes, among others, subcutaneous, intravenous, intramuscular, intra-arterial, intradermal, intraosseous, intracerebral, and intrathecal injections. The administration method will often depend upon the (poly)peptide(s) and/or immunogenic compound(s) present in the composition, and the specific type of fibrosis, autoimmune disease or an inflammatory disease to be treated and other active agents that may be contained in said composition. For example, the administration is preferably an intramuscular or an intradermal injection if the immunogenic compound is a nucleic acid as defined above, the oral/nasal administration being particularly preferred if said nucleic acid is cloned into a viral vector. Alternatively, the administration is preferably an intramuscular, an intradermal or an oral administration if the (poly)peptide and/or immunogenic compound is a (poly)peptide as defined above or if it is loaded in/on a nanoparticle as described herein. Yet, still alternatively, the administration is preferably an oral administration if the (poly)peptide and/or immunogenic compound is delivered in the form of a gut bacterium as defined above, notably if the gut bacterium is in the form of probiotics.

The (poly)peptides and/or immunogenic compounds according to the invention can further be encapsulated so as to facilitate their administration to the subject in need thereof. For example, those may be encapsulated into peptide nanocarriers (preferable if the immunogenic compound is a nucleic acid or a (poly)peptide), into virosomes (preferable if the immunogenic compound is a nucleic acid or a (poly)peptide), or into lipid-based carrier systems such as liposome-polycation-DNA complex (preferable if the immunogen is a nucleic acid or a (poly)peptide) (Trovato M, De Berardinis P. Novel antigen delivery systems. World J Virol. 2015 Aug. 12; 4(3):156-68; Saade F, Petrovsky N. Technologies for enhanced efficacy of DNA vaccines. Expert Rev Vaccines. 2012 February; 11(2):189-209; Li et al., Peptide Vaccine: Progress and Challenges. Vaccines (Basel). 2014 Jul. 2; 2(3):515-36).

The medicament may be administered once or more than once, so as to achieve the desired effect. In a preferred embodiment, the medicament is administered repeatedly, at least twice, and preferably more than twice. This can be done over an extended period of time, such as weekly, every other week, monthly, yearly, or even several years after the first administration to ensure that the subject is properly immunized.

According to one embodiment, an (poly)peptide or an immunogenic compound according to the invention may be used for the preparation of a composition and/or of an pharmaceutical composition for preventing or treating fibrosis, an autoimmune disease and/or an inflammatory disease in a subject in need thereof.

Preferably, the medicament, i.e. the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention, is used for preventing and/or treating fibrosis. Accordingly, the present invention provides a method for ameliorating, reducing, preventing and/or treating fibrosis.

The term “fibrosis”, as used herein, refers to the formation of excess fibrous connective tissue in an organ or tissue in a reparative or reactive process. In particular, the term “fibrosis” refers to the pathological state of excess deposition of fibrous tissue, in particular the pathological accumulation of extracellular matrix (ECM) proteins. Fibrosis acts to deposit connective tissue, which can obliterate the architecture and function of the underlying organ or tissue.

Preferably, the fibrosis is selected from the group consisting of: a fibrosis of the lung, a fibrosis of the liver, a gastrointestinal fibrosis, a fibrosis of the heart, a fibrosis of the brain, a fibrosis of the skin, a fibrosis of a joint, such as shoulder and/or knee, a fibrosis of hands or feet, a fibrosis of the eye, a fibrosis of bone marrow and an allograft fibrosis.

More preferably, the fibrosis is a fibrosis of the lung or of the liver. Pulmonary fibrosis includes a number of conditions that cause interstitial lung damage, followed by fibrosis and eventually loss of lung elasticity. These conditions lead to symptoms such as persistent cough, chest pain, difficulty breathing and fatigue. Pulmonary fibrosis may occur as a secondary condition in various other diseases, but in many cases the underlying cause is not clear and the term idiopathic pulmonary fibrosis is used. Liver cirrhosis refers to the scar tissue and nodules that replace liver tissue and disrupt liver function and may be caused by, for example, alcoholism, fatty liver disease, hepatitis B or hepatitis C.

It is also preferred that the fibrosis is selected from the group consisting of nonalcoholic steatohepatitis (NASH), cirrhosis, biliary atresia, schistosomiasis-induced hepatic fibrosis, systemic sclerosis, idiopathic pulmonary fibrosis (IPF), cystic fibrosis, radiation-induced lung injury, proliferative vitreoretinopathy, fibrosis in chronic TNBS colitis, fibrosis in Crohn's disease, allograft fibrosis in organ transplantation, atrial fibrosis, endomyocardial fibrosis, old myocardial fibrosis, glial scar, arthrofibrosis, mediastinal fibrosis, myelofibrosis, nephrogenic systemic fibrosis, progressive massive fibrosis, and retroperitoneal fibrosis. As far as those indications do not specifically relate to a fibrosis as such, it is preferred to prevent and/or treat the fibrosis associated with said indication.

More preferably, the fibrosis is selected the group consisting of from nonalcoholic steatohepatitis (NASH), idiopathic pulmonary fibrosis (IPF), proliferative vitreoretinopathy, fibrosis in chronic TNBS colitis, and allograft fibrosis in organ transplantation. As far as those indications do not specifically relate to a fibrosis as such, it is preferred to prevent and/or treat the fibrosis associated with said indication.

Most preferably, the disease/disorder to be treated and/or prevented is nonalcoholic steatohepatitis (NASH), in particular fibrosis associated with NASH. NASH is the most extreme form of non-alcoholic fatty liver disease. Non-alcoholic fatty liver disease begins as fatty accumulation in the liver (hepatic steatosis), which may progress to non-alcoholic steatohepatitis (NASH), a state in which steatosis is combined with an inflammatory disease and fibrosis (steatohepatitis). NASH is a progressive disease: over a 10-year period, up to 20% of patients with NASH will develop cirrhosis of the liver, and 10% will suffer death related to liver disease. IL13RA2 was found to be expressed in hepatic stellate cells (HSC) in sinusoidal lesions of the liver in patients suffering from NASH—whereas IL13RA2 is not found in normal and fatty liver tissues (Shimamura T, Fujisawa T, Husain S R, Kioi M, Nakajima A, Puri R K. Novel role of IL-13 in fibrosis induced by nonalcoholic steatohepatitis and its amelioration by IL-13R-directed cytotoxin in a rat model.) Immunol. 2008 Oct. 1; 181(7):4656-65).

Preferably, the medicament, i.e. the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention, is used for preventing and/or treating an autoimmune disease/disorder. Accordingly, the present invention provides a method for ameliorating, reducing, preventing and/or treating an autoimmune disease/disorder.

The term “autoimmune disease” or “autoimmune disorder” refers to a condition when the immune system attacks and destroys healthy body tissue. In particular, it refers to an abnormal immune response to a normal/healthy body tissue. There are at least 80 distinct types of autoimmune diseases. Nearly any tissue/body part may be involved.

It is also preferred that, the medicament, i.e. the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention, is used for preventing and/or treating an inflammatory disease. Accordingly, the present invention provides a method for ameliorating, reducing, preventing and/or treating an inflammatory disease. Preferably, the disease/disorder to be treated and/or prevented is allergic inflammation.

The term “inflammation”, as used herein, refers to a protective response of the body involving immune cells, blood vessels, and molecular mediators. The function of inflammation is to eliminate the initial cause of cell injury, clear out necrotic cells and tissues damaged from the original insult and the inflammatory process, and initiate tissue repair. In particular, inflammation is a localized reaction that produces redness, warmth, swelling, and/or pain as a result of infection, irritation, or injury. Inflammation is one of the body's natural defenses against infections from bacteria, viruses, and other invaders. Inflammation is usually a process in which the body produces and deploys white blood cells to flush out and destroy any foreign bodies in the bloodstream. However, there are certain conditions or situations in which the body can trigger inflammatory responses without any invaders being present. These conditions are typically known as “inflammatory diseases”.

The term “allergy” or “allergic”, as used herein, refers to a conditions caused by hypersensitivity of the immune system to something in the environment that usually causes little or no problem in most people. In other words, an allergy occurs when a person's immune system reacts to substances in the environment that do not bother most people. These substances are known as allergens. In particular, an allergy is a (chronic) condition involving an abnormal reaction to an ordinarily harmless substance called an allergen.

It is also preferred that the medicament, i.e. the (poly)peptide for use according to the present invention, the immunogenic compound for use according to the present invention, the nanoparticle for use according to the present invention, the cell for use according to the present invention, the nucleic acid for use according to the present invention, the host cell for use according to the present invention, or the pharmaceutical composition for use according to the present invention, is used for preventing and/or treating asthma. Accordingly, the present invention provides a method for ameliorating, reducing, preventing and/or treating asthma. Preferably, the disease/disorder to be treated and/or prevented is allergic asthma.

Asthma is a common (chronic) inflammatory disease of the airways of the lungs, in particular with increased sensitivity of the bronchia against a variety of stimuli. Symptoms include episodes of wheezing, coughing, chest tightness, and shortness of breath.

As described above, the present invention to also provides a novel approach in targeting IL13RA2 overexpressing cells and/or cells, which express IL13RA2 solely under pathological conditions, such as in fibrosis, autoimmune diseases/disorders and/or inflammatory diseases. Accordingly, the present invention also provides a method for eliciting an immune response against cells (over)expressing IL13RA2 in a subject comprising administering to the subject
the (poly)peptide according to the present invention as described above;
the immunogenic compound according to the present invention as described above;
the nanoparticle according to the present invention as described above;
the cell according to the present invention as described above;
the nucleic acid according to the present invention as described above;
the host cell according to the present invention as described above;
the pharmaceutical composition according to the present invention as described above; and/or
the combination according to the present invention as described above.

As a specific example, hepatic stellate cells expressing IL13RA2 in NASH patients may be eliminated in order to treat (fibrosis associated with) NASH. Namely, IL13RA2 was reported to be expressed in hepatic stellate cells (HSC) in sinusoidal lesions of the liver in patients suffering from non-alcoholic steatohepatitis (NASH)—but not in normal and fatty liver tissues (Shimamura T, Fujisawa T, Husain S R, Kioi M, Nakajima A, Puri R K. Novel role of IL-13 in fibrosis induced by nonalcoholic steatohepatitis and its amelioration by IL-13R-directed cytotoxin in a rat model. J Immunol. 2008 Oct. 1; 181(7):4656-65).

Combination Therapy

The administration of the (poly)peptide according to the present invention, the immunogenic compound according to the present invention, the nanoparticle according to the present invention, the cell according to the present invention, the nucleic acid according to the present invention, the host cell according to the present invention, and the pharmaceutical composition according to the present invention, in particular in the methods and uses according to the invention, can be carried out alone or in combination with a co-agent useful for treating and/or preventing fibrosis, an autoimmune disease or an inflammatory disease, such as an anti-fibrotic agent, an anti-allergic agent or an anti-inflammatory agent, respectively.

Said co-agent is thus preferably capable of preventing and/or treating the same type of disease as the one for which the (poly)peptide according to the invention is used. Preferred examples of an anti-fibrotic agent, an anti-allergic agent and an anti-inflammatory agent are described above.

The anti-fibrotic agent, the anti-allergic agent or the anti-inflammatory agent can also be administered in association with the (poly)peptide according to the present invention, the immunogenic compound according to the present invention, the nanoparticle according to the present invention, the cell according to the present invention, the nucleic acid according to the present invention, the host cell according to the present invention, or the pharmaceutical composition according to the present invention, either at about the same time or consecutively as described herein and in the same or distinct pharmaceutical forms.

Thus, in another aspect, the invention relates to a composition of the invention and at least one anti-fibrotic agent, anti-allergic agent or anti-inflammatory agent as described above, as a combined preparation for a simultaneous, separate, or sequential administration. In other terms, the invention proposes a combined use of the composition the invention and least one anti-fibrotic agent, anti-allergic agent or anti-inflammatory agent as described above, for a simultaneous, separate, or sequential administration.

Moreover, the present invention also provides the combination of (at least) two distinct (poly)peptides according to the present invention as described herein. Preferably (i) the (poly)peptide comprising or consisting of an amino acid sequence according to SEQ ID NO: 31 and (ii) any other (poly)peptide according to the present invention, namely, an (poly)peptide comprising or consisting of an amino acid sequence according to any one of SEQ ID NOs 1 to 30, 32 to 242, 267 to 274 and 279 are combined. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 1. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 2. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 3. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 4. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 5. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 6. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 7. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 8. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 9. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 10. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 11. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 12. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 13. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 14. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 15. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 16. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 17. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 18. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 19. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 20. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 21. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 22. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 23. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 24. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 25. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 26. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 27. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 28. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 29. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 30. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 32. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 33. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 34. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 35. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 36. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 37. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 38. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 39. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 40. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 41. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 42. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 43. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 44. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 45. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 46. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 47. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 48. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 49. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 50. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 51. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 52. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 53. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 54. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 55. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 56. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 57. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 58. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 59. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 60. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 61. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 62. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 63. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 64. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 65. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 66. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 67. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 68. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 69. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 70. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 71. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 72. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 73. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 74. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 75. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 76. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 77. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 78. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 79. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 80. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 81. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 82. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 83. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 84. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 85. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 86. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 87. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 88. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 89. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 90. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 91. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 92. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 93. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 94. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 95. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 96. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 97. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 98. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 99. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 100. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 101. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 102. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 103. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 104. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 105. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 106. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 107. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 108. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 109. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 110. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 111. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 112. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 113. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 114. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 115. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 116. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 117. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 118. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 119. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 120. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 121. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 122. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 123. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 124. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 125. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 126. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 127. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 128. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 129. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 130. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 131. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 132. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 133. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 134. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 135. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 136. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 137. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 138. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 139. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 140. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO:141. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 142. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 143. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 144. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 145. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 146. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 147. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 148. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 149. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 150. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 151. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 152. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 153. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 154. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 155. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 156. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 157. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 158. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 159. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 160. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 161. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 162. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO:163. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 164. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 165. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 166. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 167. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 168. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 169. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 170. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 171. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 172. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 173. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 174. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 175. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 176. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 177. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 178. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 179. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 180. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 181. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 182. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 183. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 184. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 185. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 186. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 187. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 188. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 189. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 190. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 191. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 192. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 193. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 194. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO:195. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 196. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 197. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 198. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 199. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 200. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 201. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 202. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 203. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 204. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 205. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 206. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 207. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 208. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 209. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 210. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 211. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 212. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 213. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 214. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 215. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 216. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 217. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 218. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 219. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 220. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 221. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 222. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 223. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 224. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 225. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 226. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 227. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 228. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 229. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 230. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 231. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 232. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 233. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 234. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 235. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 236. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 237. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 238. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 239. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 240. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 241. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 242. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 267. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 268. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO:269. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 270. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 271. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 272. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 273. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 274. For example, the combination of (at least) two (poly)peptides according to the present invention may comprise (i) the (poly)peptide comprising or consisting of an amino acid sequence according to SEQ ID NO: 31 and (ii) the (poly)peptide comprising or consisting of an amino acid sequence according to SEQ ID NO: 279.

It is also preferred that (i) the (poly)peptide comprising or consisting of an amino acid sequence according to SEQ ID NO: 192 and (ii) any other (poly)peptide according to the present invention, namely, an (poly)peptide comprising or consisting of an amino acid sequence according to any one of SEQ ID NOs 1 to 191, 193 to 242, 267 to 274 and 279 are combined. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 1. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 2. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 3. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 4. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 5. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 6. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 7. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 8. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 9. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 10. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 11. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 12. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 13. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 14. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 15. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 16. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 17. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 18. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 19. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 20. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 21. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 22. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 23. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 24. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 25. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 26. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 27. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 28. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 29. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 30. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 31. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 32. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 33. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 34. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 35. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 36. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 37. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 38. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 39. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 40. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 41. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 42. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 43. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 44. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 45. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 46. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 47. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 48. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 49. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 50. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 51. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 52. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 53. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 54. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 55. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 56. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 57. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 58. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 59. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 60. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 61. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 62. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 63. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 64. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 65. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 66. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 67. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 68. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 69. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 70. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 71. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 72. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 73. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 74. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 75. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 76. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 77. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 78. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 79. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 80. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 81. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 82. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 83. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 84. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 85. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 86. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 87. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 88. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 89. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 90. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 91. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 92. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 93. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 94. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 95. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 96. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 97. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 98. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 99. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 100. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 101. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 102. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 103. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 104. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 105. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 106. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 107. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 108. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 109. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 110. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 111. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 112. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 113. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 114. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 115. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 116. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 117. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 118. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 119. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 120. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 121. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 122. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 123. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 124. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 125. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 126. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 127. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 128. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 129. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 130. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 131. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 132. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 133. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 134. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 135. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 136. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 137. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 138. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 139. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 140. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO:141. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 142. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 143. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 144. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 145. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 146. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 147. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 148. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 149. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 150. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 151. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 152. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 153. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 154. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 155. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 156. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 157. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 158. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 159. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 160. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 161. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 162. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO:163. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 164. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 165. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 166. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 167. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 168. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 169. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 170. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 171. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 172. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 173. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 174. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 175. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 176. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 177. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 178. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 179. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 180. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 181. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 182. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 183. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 184. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 185. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 186. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 187. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 188. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 189. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 190. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 191. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 193. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 194. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO:195. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 196. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 197. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 198. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 199. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 200. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 201. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 202. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 203. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 204. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 205. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 206. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 207. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 208. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 209. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 210. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 211. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 212. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 213. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 214. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 215. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 216. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 217. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 218. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 219. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 220. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 221. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 222. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 223. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 224. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 225. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 226. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 227. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 228. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 229. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 230. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 231. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 232. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 233. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 234. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 235. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 236. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 237. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 238. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 239. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 240. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 241. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 242. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 267. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 268. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO:269. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 270. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 271. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 272. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 273. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 274. For example, the combination of (at least) two (poly)peptides according to the present invention may comprise (i) the (poly)peptide comprising or consisting of an amino acid sequence according to SEQ ID NO: 192 and (ii) the (poly)peptide comprising or consisting of an amino acid sequence according to SEQ ID NO: 279.

It is also preferred that (i) the (poly)peptide comprising or consisting of an amino acid sequence according to SEQ ID NO: 279 and (ii) any other (poly)peptide according to the present invention, namely, an (poly)peptide comprising or consisting of an amino acid sequence according to any one of SEQ ID NOs 1 to 242 and 267 to 274 are combined. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 1. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 2. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 3. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 4. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 5. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 6. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 7. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 8. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 9. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 10. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 11. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 12. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 13. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 14. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 15. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 16. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 17. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 18. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 19. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 20. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 21. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 22. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 23. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 24. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 25. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 26. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 27. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 28. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 29. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 30. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 31. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 32. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 33. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 34. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 35. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 36. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 37. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 38. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 39. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 40. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 41. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 42. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 43. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 44. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 45. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 46. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 47. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 48. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 49. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 50. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 51. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 52. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 53. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 54. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 55. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 56. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 57. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 58. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 59. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 60. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 61. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 62. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 63. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 64. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 65. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 66. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 67. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 68. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 69. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 70. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 71. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 72. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 73. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 74. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 75. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 76. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 77. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 78. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 79. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 80. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 81. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 82. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 83. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 84. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 85. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 86. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 87. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 88. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 89. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 90. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 91. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 92. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 93. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 94. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 95. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 96. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 97. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 98. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 99. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 100. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 101. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 102. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 103. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 104. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 105. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 106. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 107. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 108. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 109. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 110. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 111. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 112. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 113. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 114. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 115. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 116. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 117. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 118. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 119. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 120. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 121. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 122. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 123. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 124. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 125. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 126. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 127. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 128. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 129. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 130. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 131. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 132. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 133. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 134. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 135. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 136. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 137. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 138. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 139. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 140. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO:141. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 142. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 143. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 144. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 145. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 146. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 147. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 148. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 149. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 150. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 151. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 152. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 153. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 154. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 155. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 156. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 157. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 158. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 159. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 160. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 161. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 162. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO:163. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 164. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 165. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 166. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 167. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 168. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 169. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 170. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 171. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 172. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 173. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 174. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 175. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 176. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 177. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 178. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 179. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 180. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 181. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 182. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 183. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 184. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 185. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 186. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 187. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 188. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 189. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 190. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 191. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 192. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 193. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 194. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO:195. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 196. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 197. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 198. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 199. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 200. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 201. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 202. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 203. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 204. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 205. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 206. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 207. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 208. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 209. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 210. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 211. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 212. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 213. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 214. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 215. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 216. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 217. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 218. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 219. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 220. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 221. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 222. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 223. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 224. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 225. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 226. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 227. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 228. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 229. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 230. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 231. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 232. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 233. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 234. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 235. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 236. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 237. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 238. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 239. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 240. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 241. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 242. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 267. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 268. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO:269. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 270. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 271. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 272. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 273. For example, the combination of (at least) two (poly)peptides according to the present invention may comprise (i) the (poly)peptide comprising or consisting of an amino acid sequence according to SEQ ID NO: 279 and (ii) the (poly)peptide comprising or consisting of an amino acid sequence according to SEQ ID NO: 274.

Most preferably (i) the (poly)peptide comprising or consisting of an amino acid sequence according to SEQ ID NO: 31 or 279 and (ii) the (poly)peptide comprising or consisting of an amino acid sequence according to SEQ ID NO: 192 are combined.

Moreover, the (poly)peptide for use according to the present invention comprising a sequence variant may also be combined with a (poly)peptide for use according to the present invention comprising the (corresponding) human IL13RA2 epitope (e.g., as described above regarding the peptide “families”). Thereby, selection of T-cell clones, which are very efficient against the tumor, is obtained/supported. In particular, the (poly)peptide according to the present invention comprising the sequence variant and the (poly)peptide according to the present invention comprising the (corresponding) human IL13RA2 epitope may be co-administered. Such co-administration may be at about the same time (simultaneously) or consecutively, whereby in consecutive administration it is preferred that the (poly)peptide according to the present invention comprising the sequence variant is administered first and the (poly)peptide according to the present invention comprising the corresponding (human) tumor antigen epitope is administered thereafter. In particular, the (poly)peptide according to the present invention comprising a sequence variant may be administered first, and the (poly)peptide according to the present invention comprising the corresponding (human) tumor antigen epitope may be used as (re)boost. For example, a (poly)peptide comprising or consisting of an amino acid sequence according to SEQ ID NO: 31 may be combined with a (poly)peptide comprising or consisting of an amino acid sequence according to SEQ ID NO: 263. In another example, a (poly)peptide comprising or consisting of an amino acid sequence according to SEQ ID NO: 192 may be combined with a (poly)peptide comprising or consisting of an amino acid sequence according to SEQ ID NO: 245.

The peptides, which are to be combined, such as a (poly)peptide for use according to the present invention comprising a sequence variant and a (poly)peptide for use according to the present invention comprising the corresponding (human) IL13RA2 epitope or (b) at least two distinct (poly)peptides according to the present invention comprising a sequence variant, may be administered
in the same immunogenic compound according to the present invention or in distinct immunogenic compounds according to the present invention,
(loaded) in the same nanoparticle according to the present invention or in distinct nanoparticles according to the present invention,
(loaded) in the same cell according to the present invention or in distinct cells according to the present invention,
(encoded by) the same nucleic acid according to the present invention or by distinct nucleic acids according to the present invention,
(expressed by) the same host cell according to the present invention or by distinct host cells according to the present invention, or
(comprised) in the same pharmaceutical composition according to the present invention or in distinct pharmaceutical composition according to the present invention.

For example, the present invention provides a combination of
(i) a first (poly)peptide for use according to the present invention, and
(ii) a second (poly)peptide for use according to the present invention

for use in the prevention and/or treatment of fibrosis, an autoimmune disease and/or an inflammatory disease.

A particularly preferred combination according to the present invention comprises
(i) a (poly)peptide for use according to the present invention comprising or consisting of an amino acid sequence as set forth in SEQ ID NO: 31, and
(ii) a (poly)peptide for use according to the present invention comprising or consisting of an amino acid sequence as set forth in SEQ ID NO: 192

for use in the prevention and/or treatment of fibrosis, an autoimmune disease and/or an inflammatory disease.

Another particularly preferred combination according to the present invention comprises
(i) a (poly)peptide for use according to the present invention comprising or consisting of an amino acid sequence as set forth in SEQ ID NO: 279, and
(ii) a (poly)peptide for use according to the present invention comprising or consisting of an amino acid sequence as set forth in SEQ ID NO: 192

for use in the prevention and/or treatment of fibrosis, an autoimmune disease and/or an inflammatory disease.

For example, the present invention provides a combination of
(i) an immunogenic compound for use according to the present invention comprising a first (poly)peptide according to the present invention, and
(ii) an immunogenic compound for use according to the present invention comprising a second (poly)peptide according to the present invention

for use in the prevention and/or treatment of fibrosis, an autoimmune disease and/or an inflammatory disease.

A particularly preferred combination according to the present invention comprises
(i) an immunogenic compound for use according to the present invention comprising an (poly)peptide comprising or consisting of an amino acid sequence as set forth in SEQ ID NO: 31, and
(ii) an immunogenic compound for use according to the present invention comprising an (poly)peptide comprising or consisting of an amino acid sequence as set forth in SEQ ID NO: 192

for use in the prevention and/or treatment of fibrosis, an autoimmune disease and/or an inflammatory disease.

Another particularly preferred combination according to the present invention comprises
(i) an immunogenic compound for use according to the present invention comprising an (poly)peptide comprising or consisting of an amino acid sequence as set forth in SEQ ID NO: 279, and
(ii) an immunogenic compound for use according to the present invention comprising an (poly)peptide comprising or consisting of an amino acid sequence as set forth in SEQ ID NO: 192

for use in the prevention and/or treatment of fibrosis, an autoimmune disease and/or an inflammatory disease.

For example, the present invention provides a combination of
(i) a nanoparticle for use according to the present invention comprising a first (poly)peptide according to the present invention, and
(ii) a nanoparticle for use according to the present invention comprising a second (poly)peptide according to the present invention

for use in the prevention and/or treatment fibrosis, an autoimmune disease and/or an inflammatory disease.

A particularly preferred combination according to the present invention comprises
(i) a nanoparticle for use according to the present invention comprising an (poly)peptide comprising or consisting of an amino acid sequence as set forth in SEQ ID NO: 31, and
(ii) a nanoparticle for use according to the present invention comprising an (poly)peptide comprising or consisting of an amino acid sequence as set forth in SEQ ID NO: 192

for use in the prevention and/or treatment of fibrosis, an autoimmune disease and/or an inflammatory disease.

Another particularly preferred combination according to the present invention comprises
(i) a nanoparticle for use according to the present invention comprising an (poly)peptide comprising or consisting of an amino acid sequence as set forth in SEQ ID NO: 279, and
(ii) a nanoparticle for use according to the present invention comprising an (poly)peptide comprising or consisting of an amino acid sequence as set forth in SEQ ID NO: 192

for use in the prevention and/or treatment of fibrosis, an autoimmune disease and/or an inflammatory disease.

For example, the present invention provides a combination of
(i) a nucleic acid for use according to the present invention comprising a polynucleotide encoding a first (poly)peptide according to the present invention and
(ii) a nucleic acid for use according to the present invention comprising a polynucleotide encoding a first (poly)peptide according to the present invention

for use in the prevention and/or treatment of fibrosis, an autoimmune disease and/or an inflammatory disease.

A particularly preferred combination according to the present invention comprises
(i) a nucleic acid for use according to the present invention comprising a polynucleotide encoding the amino acid sequence as set forth in SEQ ID NO: 31 and
(ii) a nucleic acid for use according to the present invention comprising a polynucleotide encoding the amino acid sequence as set forth in SEQ ID NO: 192

for use in the prevention and/or treatment of fibrosis, an autoimmune disease and/or an inflammatory disease.

Another particularly preferred combination according to the present invention comprises
(i) a nucleic acid for use according to the present invention comprising a polynucleotide encoding the amino acid sequence as set forth in SEQ ID NO: 279 and
(ii) a nucleic acid for use according to the present invention comprising a polynucleotide encoding the amino acid sequence as set forth in SEQ ID NO: 192

for use in the prevention and/or treatment of fibrosis, an autoimmune disease and/or an inflammatory disease.

In general, the distinct components administered in a combination therapy as described herein may be administered simultaneously, in particular at about the same time, or sequentially/consecutively.

Preferably, the (active) components, which are to be combined, are administered at about the same time, in particular simultaneously. More preferably, the (active) components which are administered at about the same time, in particular simultaneously, are preferably administered in the same form (i.e., in the same type of formulation, e.g., as nanoparticles, as pharmaceutical compositions, etc.).

“At about the same time”, as used herein, means in particular simultaneous administration or that directly after administration of (i) the first component, (ii) the second component is administered or directly after administration of (ii) the second component (i) the first component is administered. The skilled person understands that “directly after” includes the time necessary to prepare the second administration—in particular the time necessary for exposing and disinfecting the location for the second administration as well as appropriate preparation of the “administration device” (e.g., syringe, pump, etc.). Simultaneous administration also includes if the periods of administration of (i) the first component and of (ii) the second component overlap or if, for example, one component is administered over a longer period of time, such as 30 min, 1 h, 2 h or even more, e.g. by infusion, and the other component is administered at some time during such a long period. Administration of (i) the first component and of (ii) the second component at about the same time is in particular preferred if different routes of administration and/or different administration sites are used.

It is also preferred that the (active) components, which are to be combined, are administered consecutively. In more general, it is preferred that the first component and the second component are administered consecutively, wherein the (active) components are preferably administered in the same form (i.e., in the same type of formulation, e.g., as nanoparticles, as pharmaceutical compositions, etc.).

This means that (i) the first component is administered before or after (ii) the second component. In consecutive administration, the time between administration of the first component and administration of the second component is preferably no more than one week, more preferably no more than 3 days, even more preferably no more than 2 days and most preferably no more than 24 h. It is particularly preferred that (i) the first component and (ii) the second component are administered at the same day with the time between administration of the first component and administration of the second component being preferably no more than 6 hours, more preferably no more than 3 hours, even more preferably no more than 2 hours and most preferably no more than 1 h.

Preferably, the (active) components, which are to be combined, are administered via the same or distinct routes of administration.

Preferably, (i) the first component and (ii) the second component are administered via the same route of administration. In more general, it is preferred that the first component and the second component are administered via the same route of administration, wherein the (active) components are preferably administered in the same form (i.e., in the same type of formulation, e.g., as nanoparticles, as pharmaceutical compositions, etc.).

It is also preferred that the (active) components, which are to be combined, are administered via distinct routes of administration. In more general, it is preferred that the first component and the second component are administered via distinct routes of administration, wherein the (active) components are preferably administered in the same form (i.e., in the same type of formulation, e.g., as nanoparticles, as pharmaceutical compositions, etc.).

Preferably, the (active) components, which are to be combined, are comprised in the same or distinct compositions.

Preferably, the (active) components are comprised in the same composition. In more general, it is preferred that the first component and the second component are comprised in the same composition, wherein the (active) components are preferably administered in the same form (i.e., in the same type of formulation, e.g., as nanoparticles, etc.).

It is also preferred that the (active) components are comprised in distinct compositions. In more general, it is preferred that the first component and the second component are comprised in distinct compositions, wherein the (active) components, which are to be combined, are preferably administered in the same form (i.e., in the same type of formulation, e.g., as nanoparticles, etc.).

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows the general protocol for the validation of the proof-of-concept of an antigen-based immunotherapy targeting IL13RA2.

FIG. 2: shows a schematic view of the immunization scheme. d: day.

FIG. 3: shows ELISPOT-IFNγ results for group 1 (IL13RA2-B) and group 2 (IL13RA2-A). The peptide used for vaccination (in between brackets under each group) and the stimulus used in the ELISPOT culture (X-axis) are indicated on the graphs. (A) Number of specific ELISPOT-IFNγ spots (medium condition subtracted). Each dot represents the average value for one individual/mouse from the corresponding condition quadruplicate. (B) For each individual, the level of specific ELISPOT-IFNγ response is compared to the ConA stimulation (value: 100%). Statistical analysis: paired t-test for intra-group comparison and unpaired t-test for inter-group comparison; *p<0.05.

FIG. 4: shows the results of Example 3.

FIG. 5: shows the results of Example 4.

FIG. 6: shows for Example 6 ELISPOT results for HLA-A2 transgenic mice vaccinated with the peptide IL13R2A-L as indicated in the figure and cross-reactivity with the human corresponding peptide IL13RA2-H. For each group the normalized number of spot-forming cells (SFC) is shown.

FIG. 7 shows for Example 7 in vitro affinity for peptides ILI 3RA2-B and IL13RA2-L in comparison to the corresponding human peptide IL13RA2-H and to the comparative peptide 1A9V.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

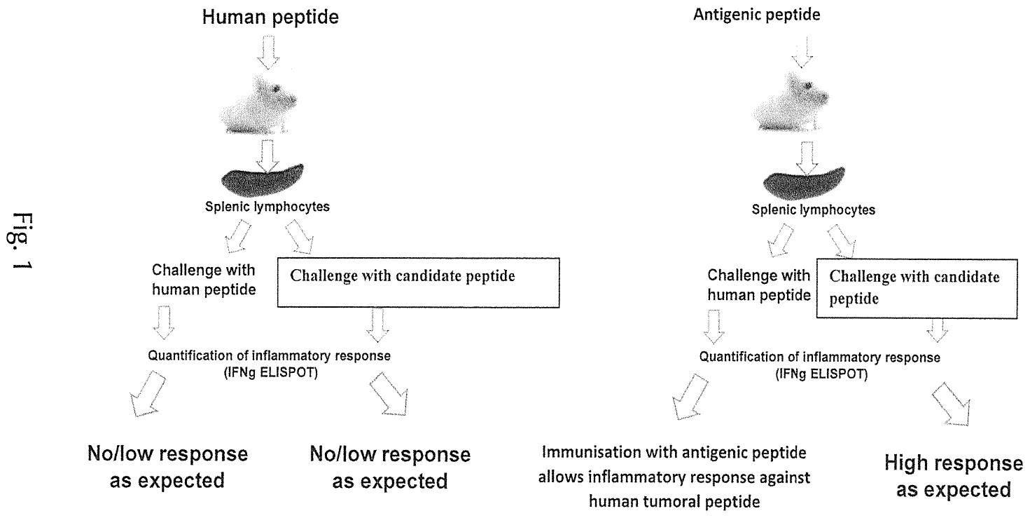

Examples 1 and 2 are both linked to the general protocol described in FIG. 1.

### Example 1: Identification of a Candidate (Poly)Peptide Having Superior Affinity to the HLA-A*0201 Allele

This Example provides evidence that the (poly)peptide of sequence SEQ ID NO: 31 («FLPFGFILV» also referred herein as IL13RA2-B) has high affinity to the HLA-A*0201 allele, whereas the corresponding reference human peptide derived from IL13RA2 («WLPFGFILI», SEQ ID No 263, also referred herein as IL13RA2-H) has low affinity.

A. Materials and Methods

A1. Measuring the Affinity of the Peptide to T2 Cell Line.

The experimental protocol is similar to the one that was validated for peptides presented by the HLA-A*0201 (Tourdot et al., A general strategy to enhance immunogenicity of low-affinity HLA-A2.1-associated peptides: implication in the identification of cryptic tumor epitopes. Eur J Immunol. 2000 December; 30(12):3411-21). Affinity measurement of the peptides is achieved with the human tumoral cell T2 which expresses the HLA-A*0201 molecule, but which is TAP1/2 negative and incapable of presenting endogenous peptides.

T2 cells (2.10⁵ cells per well) are incubated with decreasing concentrations of peptides from 100 μM to 0.1 μM in a AIMV medium supplemented with 100 ng/μl of human β2m at 37° C. for 16 hours. Cells are then washed two times and marked with the anti-HLA-A2 antibody coupled to PE (clone BB7.2, BD Pharmagen).

The analysis is achieved by FACS (Guava Easy Cyte).

For each peptide concentration, the geometric mean of the labelling associated with the peptide of interest is substracted from background noise and reported as a percentage of the geometric mean of the HLA-A*0202 labelling obtained for the reference peptide HIV pol 589-597 at a concentration of 100 μM. The relative affinity is then determined as follows:

relative affinity=concentration of each peptide inducing 20% of expression of HLA-A*0201/concentration of the reference peptide inducing 20% of expression of HLA-A*0201.

A2. Solubilisation of Peptides

Each peptide is solubilized by taking into account the amino acid composition. For peptides which do not include any Cystein, Methionin, or Tryptophane, the addition of DMSO is possible to up to 10% of the total volume. Other peptides are resuspended in water or PBS pH7.4.

B. Results

For T2 Cells: Mean fluorescence intensity for variable peptidic concentrations: Regarding the couple IL13RA2 peptides (IL13RA2-H and IL13RA2-B), it appears that the human peptide does not bind to the HLA-A*0201 contrarily to the candidate peptide IL13RA2-B, which binds strongly to HLA-A*0201: 112.03 vs 18.64 at 100 μM; 40.77 vs 11.61 at 10 μM; 12.18 vs 9.41 at 1 μM; 9.9 vs 7.46 at 0.1 μM.

Also, IL13RA2-13 at 4.4 μM induces 20% of expression of the HLA-A*0201 (vs 100 μM for IL13RA2-H).

Similar results were obtained from a second distinct T2 cell clone.

### Example 2: Vaccination on Mice with the Candidate (Poly)Peptide Induces Improved T Cell Responses in a ELISPOT-IFNγ Assay

A. Materials and Methods

A.1 Mouse Model

The features of the model used in this project are shown in Table 4.

TABLE 4
Model features.

| | |
|---|---|
| Mouse Model | C57BL/6J B2m ^{tm1Unc}IAb^{-/-}Tg(HLA-DRA |
| | HLA-DRB1*0301)^{#Gjh} Tg(HLA-A/H2-D/B2M)^{1BPe} |
| Acronym | β/A2/DR3 |
| Description | Immunocompetent, no mouse class I and class II MHC |
| Housing | SOPF conditions (ABSL3) |
| Number of mice | 24 adults (>8 weeks of age) |

A.2. Immunization Scheme.

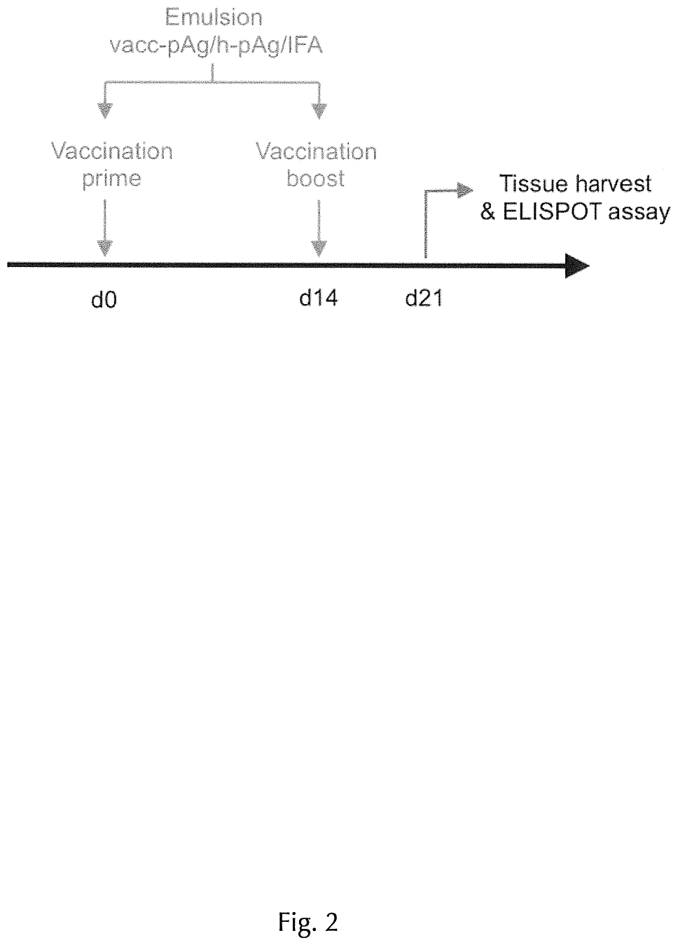

The immunization scheme is shown in FIG. 2. Briefly β/A2/DR3 mice were assigned randomly (based on mouse sex and age) to two experimental groups, each immunized with a specific vaccination peptide (vacc-pAg) combined to a common helper peptide (h-pAg) (as outlined in Table 5 below). The vacc-pAg were compared in couples (group 1 vs. group 2). Thereby, both native and optimized versions of a single peptide were compared in each wave.

TABLE 5
Experimental group composition. h-pAg: ‘helper’ peptide;
vacc-pAg: vaccination peptide. The number
of boost injections is indicated into brackets.

| | | | | | Animal |
|---|---|---|---|---|---|
| Group | Peptide (vacc-pAg) | Helper (h-pAg) | Prime | Boost | number |
| 1 | IL13RA2-B (100 μg) | HHD-DR3 (150 μg) | + | +(1X) | 6 |
| 2 | IL13RA2-H (100 μg) | HHD-DR3 (150 μg) | + | +(1X) | 6 |

The peptides were provided as follows:
couples of vacc-pAg: IL13RA2-H and IL13RA2-B; all produced and provided at a 4 mg/ml (4 mM) concentration;
h-pAg: HHD-DR3; provided lyophilized (50.6 mg; Eurogentec batch 1611166) and re-suspended in pure distilled water at a 10 mg/mL concentration;

The animals were immunized on day 0 (d0) with a prime injection, and on d14 with a boost injection. Each mouse was injected s.c. at tail base with 100 μL of an oil-based emulsion that contained:
100 μg of vacc-pAg (25 μL of 4 mg/mL stock per mouse);
150 μg of h-pAg (15 μL of 10 mg/mL stock per mouse);
10 μL of PBS to reach a total volume of 50 μL (per mouse);
Incomplete Freund's Adjuvant (IFA) added at 1:1 (v:v) ratio (50 μL per mouse).

A separate emulsion was prepared for each vacc-pAg, as follows: IFA reagent was added to the vacc-pAg/h-pAg/PBS mixture in a 15 mL tube and mixed on vortex for repeated cycles of 1 min until forming a thick emulsion.

A.3. Mouse Analysis

Seven days after the boost injection (i.e. on d21), the animals were euthanized and the spleen was harvested. Splenocytes were prepared by mechanical disruption of the organ followed by 70 μm-filtering and Ficoll density gradient purification.

The splenocytes were immediately used in an ELISPOT-IFNγ assay (Table 6). Experimental conditions were repeated in quadruplets, using 2*10⁵ total splenocytes per well, and were cultured in presence of vacc-pAg (10 μM), Concanavalin A (ConA, 2.5 μg/mL) or medium-only to assess for their capacity to secrete IFNγ. The commercial ELISPOT-IFNγ kit (Diaclone Kit Mujrine IFNγ ELISpot) was used following the manufacturer's instructions, and the assay was performed after about 16h of incubation.

TABLE 6
Setup of the ELISPOT-IFNγ assay.

| Group | Stimulus | Wells | Animal | Total |
|---|---|---|---|---|
| 1 | IL13RA2-B (10 μM) | 4 | 6 | 24 |
| | IL13RA2-H (10 μM) | 4 | 6 | 24 |
| | ConA (2.5 μg/ml) | 4 | 6 | 24 |
| | Medium | 4 | 6 | 24 |
| 2 | IL13RA2-B (10 μM) | 4 | 6 | 24 |
| | IL13RA2-H (10 μM) | 4 | 6 | 24 |
| | ConA (2.5 μg/ml) | 4 | 6 | 24 |
| | Medium | 4 | 6 | 24 |

Spots were counted on a Grand ImmunoSpot® S6 Ultimate UV Image Analyzer interfaced to the ImmunoSpot 5.4 software (CTL-Europe). Data plotting and statistical analysis were performed with the Prism-5 software (GraphPad Software Inc.).

The cell suspensions were also analyzed by flow cytometry, for T cell counts normalization. The monoclonal antibody cocktail (data not shown) was applied on the purified leucocytes in presence of Fc-block reagents targeting murine (1:10 diluted ‘anti-mCD16/CD32 CF11 clone’—internal source) Fc receptors. Incubations were performed in 96-well plates, in the dark and at 4° C. for 15-20 minutes. The cells were washed by centrifugation after staining to remove the excess of monoclonal antibody cocktail, and were re-suspended in PBS for data acquisition.

All data acquisitions were performed with an LSR-II Fortessa flow cytometer interfaced with the FACS-Diva software (BD Bioscience). The analysis of the data was performed using the FlowJo-9 software (TreeStar Inc.) using a gating strategy (not shown).

TABLE 7
FACS panel EXP-1.

| Target | Label | Clone | Provider | Dilution |
|---|---|---|---|---|
| mCD3ϵγ | FITC | 145-2C11 | Biolegend | 1/100 |
| mCD4 | PE | RM4-5 | Biolegend | 1/100 |
| mCD8α | APC | 53-6, 7 | Biolegend | 1/100 |

B. Results

A total of 14 β/A2/DR3 mice were used for this experiment (see Table 8). At time of sacrifice, the spleen T cell population was analysed by flow cytometry, showing that the large majority belonged to the CD4+ T cell subset.

TABLE 8
Individual mouse features (groups 1 & 2). Each mouse
is identified by a unique ear tag ID number.

| Mouse | | Age^{a} | Group | T cells^{b} | T4^{c} | T8^{c} | |
|---|---|---|---|---|---|---|---|
| ID | Sex | (wks) | (pAg) | (%) | (%) | (%) | Note^{d} |
| 826 | M | 14 | 1 (IL13RA2-B) | 18.6 | 72.0 | 13.7 | P1/2 |
| 827 | M | 14 | 1 (IL13RA2-B) | 21.1 | 82.5 | 8.7 | P1/2 |
| 828 | M | 14 | 1 (IL13RA2-B) | 20.9 | 78.4 | 8.6 | P1/2 |
| 829 | F | 15 | 1 (IL13RA2-B) | 23.8 | 67.0 | 17.5 | P1/2 |
| 830 | F | 15 | 1 (IL13RA2-B) | 29.2 | 73.3 | 12.5 | P1/2 |
| 831 | F | 15 | 1 (IL13RA2-B) | N.A. | N.A. | N.A. | ID tag lost |
| | | | | | | | (excluded) |
| 17 | M | 9 | 1 (IL13RA2-B) | 8.3 | 83.7 | 10.4 | P5 |
| 832 | F | 15 | 2 (IL13RA2-H) | 28.3 | 83.4 | 5.7 | P1/2 |
| 833 | F | 15 | 2 (IL13RA2-H) | N.A. | N.A. | N.A. | ID tag lost |
| | | | | | | | (excluded) |
| 834 | F | 15 | 2 (IL13RA2-H) | 27.5 | 79.7 | 7.2 | P1/2 |
| 835 | M | 13 | 2 (IL13RA2-H) | 33.8 | 84.2 | 8.5 | P1/2 |
| 836 | M | 13 | 2 (IL13RA2-H) | 31.4 | 84.7 | 6.3 | P1/2 |
| 837 | M | 15 | 2 (IL13RA2-H) | 30.8 | 83.4 | 5.4 | P1/2 |
| 18 | M | 9 | 2 (1L13RA2-H) | 11.2 | 85.9 | 9.2 | P5 |

^{a}age at onset of the vaccination protocol (in weeks);
^{b}percentage of T cells in total leukocytes;
^{c}percentage of CD4+ or CD8+ T cells in total T cells;
^{d}plate (P) number.

After plating and incubation with the appropriate stimuli, the IFNγ-producing cells were revealed and counted. The data were then normalized as a number of specific spots (the average counts obtained in the ‘medium only’ condition being subtracted) per 10⁶ total T cells.

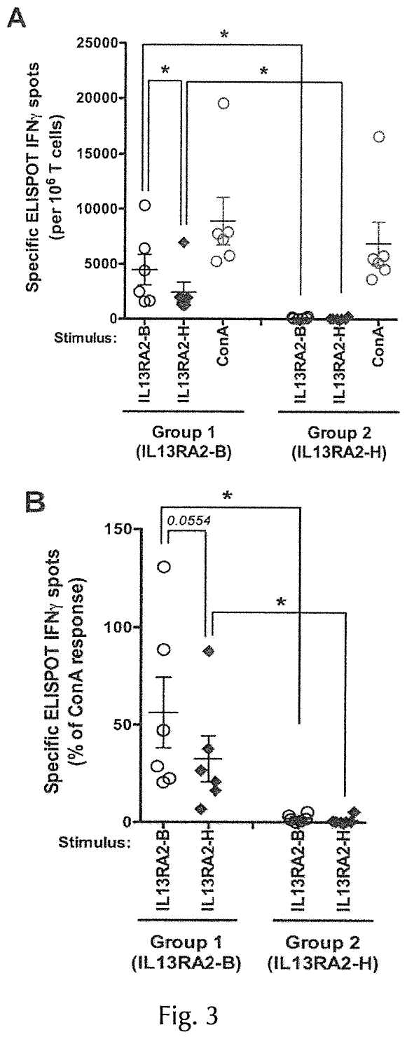

The individual average values (obtained from the quadruplicates) were next used to plot the group average values (see FIG. 3A). As the functional capacity of T cells might vary from individual to individual, the data were also expressed as the percentage of the ConA response per individual (see FIG. 3B).

Overall, vaccination with the IL13RA2-B pAg (candidate) peptide induced improved T cell responses in the ELISPOT-IFNγ assay, as compared to IL13RA2-H pA (reference human)-vaccinated animals (group 2). For group 1 (IL13RA2-B), ex vivo restimulation with the IL13RA2-B pAg promoted higher response than with the IL13RA2-H pAg. It was not the case for group 2 (IL13RA2-H). The percentage of ConA-induced response (mean+/−SEM) for each condition was as follows:
Group 1 (IL13RA2-B)/IL13RA2-B pAg: 56.3%+/−18.1
Group 1 (IL13RA2-B)/IL13RA2-H pAg: 32.3%+/−11.8
Group 2 (IL13RA2-H)/IL13RA2-B pAg: 2.0%+/−0.8
Group 2 (IL13RA2-H)/IL13RA2-H pAg: 1.1%+/−0.8

Accordingly, those results provide experimental evidence that antigen-based immunotherapy targeting IL13RA2 is able to improve T cell response in vivo and that the IL13RA2-B candidate peptide (SEQ ID NO: 31) is particularly efficient for that purpose.

### Example 3: Candidate (Poly)Peptides Having Superior Affinity to the HLA-A*0201 Allele

This Example provides further evidence that the (poly)peptide of sequence SEQ ID NO: 31 («FLPFGFILV», also referred to herein as IL13RA2-B) has high affinity to the HLA-A*0201 allele, whereas the corresponding reference human peptide derived from IL13RA2 («WLPFGFILI», SEQ ID No 263, also referred to herein as IL13RA2-H) has low affinity. Moreover, this Example provides evidence that the (poly)peptide of sequence SEQ ID NO: 192 («YLYTFLIST», also referred to herein as IL13RA2-B2) has high affinity to the HLA-A*0201 allele, whereas the corresponding reference human peptide derived from IL13RA2 («CLYTFLIST», SEQ ID NO: 245, also referred to herein as IL13RA2-H2) has low affinity.

A. Materials and Methods

A1. Measuring the Affinity of the Peptide to T2 Cell Line.

The experimental protocol was similar to the one that was validated for peptides presented by the HLA-A*0201 (Tourdot et al., A general strategy to enhance immunogenicity of low-affinity HLA-A2.1-associated peptides: implication in the identification of cryptic tumor epitopes. Eur J Immunol. 2000 December; 30(12):3411-21). Affinity measurement of the peptides was achieved with the human tumor cell T2 which expresses the HLA-A*0201 molecule, but which is TAP1/2 negative and incapable of presenting endogenous peptides.

T2 cells (2.10⁵ cells per well) were incubated with decreasing concentrations of peptides from 100 μM to 0.1 μM in a AIMV medium supplemented with 100 ng/μl of human β2m at 37° C. for 16 hours. Cells were then washed two times and marked with the anti-HLA-A2 antibody coupled to PE (clone BB7.2, BD Pharmagen).

The analysis was achieved by FACS (Guava Easy Cyte).

For each peptide concentration, the geometric mean of the labelling associated with the peptide of interest was subtracted from background noise and reported as a percentage of the geometric mean of the HLA-A*0202 labelling obtained for the reference peptide HIV pol 589-597 at a concentration of 100 μM. The relative affinity is then determined as follows:

relative affinity=concentration of each peptide inducing 20% of expression of HLA-A*0201/concentration of the reference peptide inducing 20% of expression of HLA-A*0201.

A2. Solubilisation of Peptides

Each peptide was solubilized by taking into account the amino acid composition. For peptides which do not include any Cystein, Methionin, or Tryptophane, the addition of DMSO is possible to up to 10% of the total volume. Other peptides are resuspended in water or PBS pH 7.4.

B. Results

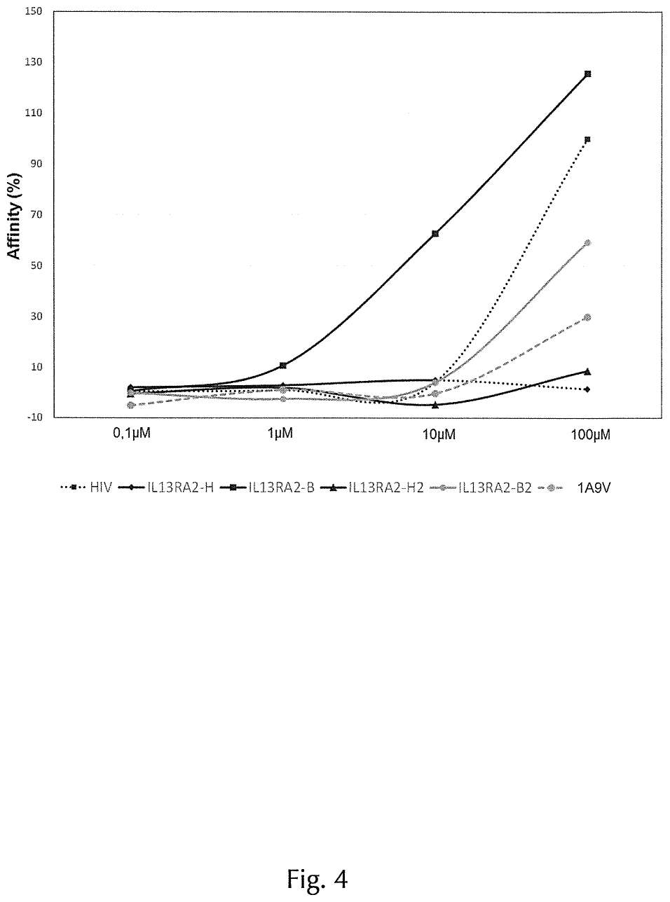

Results are shown in FIG. 4. Regarding the two couples of IL13RA2 peptides ((i) IL13RA2-H and IL13RA2-B, and (ii) IL13RA2-H2 and IL13RA2-B2), the human peptides do not bind to or show much lower affinity to HLA-A*0201, whereas the candidate peptides IL13RA2-B and IL13RA2-B2, bind strongly to HLA-A*0201. Moreover, both candidate peptides bind to HLA-A*0201 with higher affinity than the peptide “1A9V” (as described by Eguchi Junichi et al., 2006, Identification of interleukin-13 receptor alpha 2 peptide analogues capable of inducing improved antiglioma CTL responses. Cancer Research 66(11): 5883-5891). Reference peptide HIV pol 589-597 (“HIV”) served as positive control.

Similar results were obtained from a second distinct T2 cell clone.

### Example 4: Candidate (Poly)Peptide Provides In Vitro Cytotoxicity Against Cells Expressing IL13RA2

This Example provides evidence that the (poly)peptide of sequence SEQ ID NO: 31 («FLPFGFILV», also referred to herein as IL13RA2-B) provides in vitro cytotoxicity against U87 cells, which express IL13RA2. In contrast, the corresponding reference human peptide derived from IL13RA2 («WLPFGFILI», SEQ ID No 263, also referred to herein as IL13RA2-H) does not provide in vitro cytotoxicity against U87 cells.

Methods:

Briefly, CD8 T cells from mice immunized with IL13RA2-H or IL13RA2-B were used. These cells were obtained after sorting of splenocyte from immunized mice and were placed on top of U87 cells (expressing IL13RA2).

In more detail, CD3⁺ T cells were purified from splenocytes of HHD mice immunized with IL13RA2-H (WLPFGFILI, SEQ ID No 263) or IL13RA2-B (FLPFGFILV). To this end, B6 β2 m^{ko} HHD/DR3 mice were injected s.c. at tail base with 100 μL of an oil-based emulsion containing vaccination peptide plus helper peptide plus CFA (complete Freund's adjuvant), at day 0 and day 14. On d21, i.e. seven days after the boost injection, the animals were euthanized and the spleen was harvested. Splenocytes were prepared by mechanical disruption of the organ. CD3+ purification was performed using the mouse total T cells isolation kit from Miltenyi biotec using the recommended procedure. Efficient purification of cells and viability was validated by cytometry using appropriate marker for viability, CD8, CD4, CD3, and CD45.

U87-MG cells were seeded at 6×10⁵ cells/well in flat-bottomed 24-well culture plates and incubated for 24 h at 37° C. in DMEM (Dulbecco's Modified Eagle Medium) containing 10% of FCS (fetal calf serum) and antibiotics. After 24 hours, culture media were removed and replaced with media containing purified T CD3+ cells. The following ratios of T cells vs. U87-MG cells were used: 1/0.5, 1/1 and 1/5.

72 hours after co-culture of U87-MG cells and CD3+ T cells, all cells from the wells were harvested and specific U87-MG cell death was evaluated after immunostaining of CD45 negative cells with DAPI and fluorescent annexin V followed by cytometry analysis.

Results:

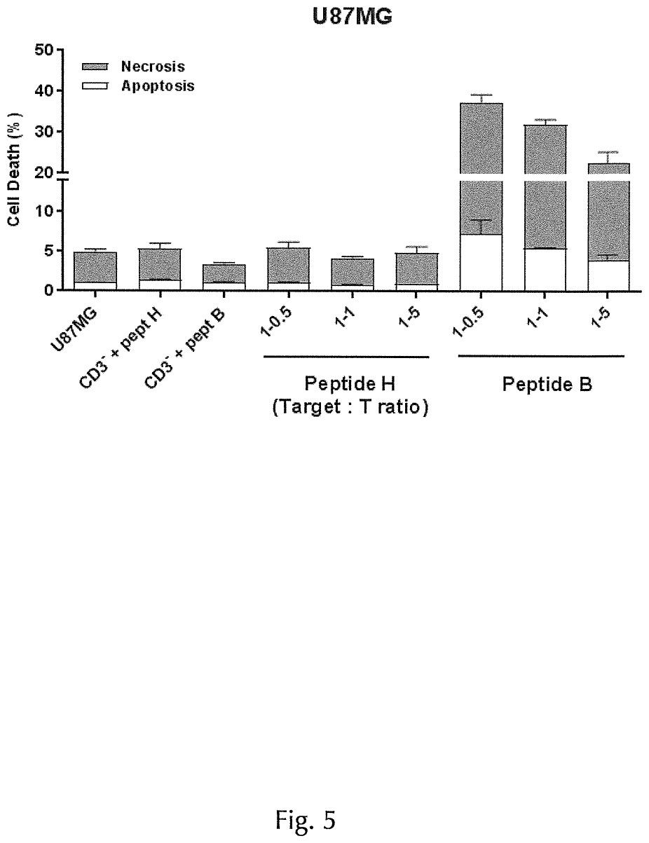

Results are shown in FIG. 5. In general, U87 cell lysis was observed after treatment with IL13 RA2-B, but not with IL13 RA2-H.

### Example 5: Candidate (Poly)Peptide has Superior Affinity to the HLA-A*0201 Allele

This Example provides evidence that the (poly)peptide of sequence SEQ ID NO: 31 («FLPFGFILV», also referred to herein as IL13RA2-B) has higher affinity to the HLA-A*0201 allele than other sequence variants of the corresponding reference human peptide derived from IL13RA2 («WLPFGFILI», SEQ ID No 263, also referred to herein as IL13RA2-H). In this experiment, the (poly)peptide of sequence SEQ ID NO: 31 was compared to
the peptide “1A9V”, as described by Eguchi Junichi et al., 2006, Identification of interleukin-13 receptor alpha 2 peptide analogues capable of inducing improved antiglioma CTL responses. Cancer Research 66(11): 5883-5891, in which the tryptophan at position 1 of SEQ ID No 263 was substituted by alanine (1A) and the isoleucine at position 9 of SEQ ID No 263 was substituted by valine (9V);
peptide “1I9A”, wherein the tryptophan at position 1 of SEQ ID No 263 was substituted by isoleucine (1I) and the isoleucine at position 9 of SEQ ID No 263 was substituted by alanine (9A); and
peptide “1F9M”, wherein the tryptophan at position 1 of SEQ ID No 263 was substituted by phenylalanine (1F) and the isoleucine at position 9 of SEQ ID No 263 was substituted by methionine (9M).

A. Materials and Methods

The experimental protocol, materials and methods correspond to those outlined in Example 3, with the only difference that the above mentioned (poly)peptides were used.

B. Results

The following in vitro binding affinities were obtained:

| Peptide | In vitro binding affinity |
|---|---|
| IL13RA2-B (SEQ ID NO: 31) | 0.49 |
| 1A9V | 3.06 |
| 1I9A | 2.22 |
| 1F9M | 2.62 |

Accordingly, the (poly)peptide according to the present invention (IL13RA2-B (SEQ ID NO: 31)) showed considerably higher binding affinity to HLA-A*0201 than all other peptides tested, whereas the peptide “1A9V”, as described by Eguchi Junichi et al., 2006, Identification of interleukin-13 receptor alpha 2 peptide analogues capable of inducing improved antiglioma CTL responses. Cancer Research 66(11): 5883-5891, showed the lowest affinity of the peptides tested.

### Example 6: Immunogenicity of IL13R2A-L in HLA-A2 Transgenic Mice and Cross-Reactivity with the Corresponding Human Peptide

A. Materials and Methods

The (poly)peptide of the present invention IL13RA2-L (SEQ ID NO: 279) and the corresponding human reference peptide IL13RA2-H (SEQ ID NO: 263) were tested in distinct groups of male and female HHD DR3 mice expressing human HLA-A2 and HLA-DR3 MHC and lacking the murine H-2 class I and class II MHCs. Groups of 5 mice (male and female) were subcutaneously injected on days 0 and 14 with 100 μg of IL13RA2-L (SEQ ID NO: 279) or IL13RA2-H (SEQ ID NO: 263), 150 μg of helper peptide (DR3) and IFA. On day 21, the mice were euthanized and splenocytes were prepared and stimulated in vitro with IL13RA2-L or the human corresponding peptide IL13RA2-H to assess their capacity to secrete IFN- as assessed by ELISpot. Concanavalin A (ConA) was used as a positive control.

B. Results

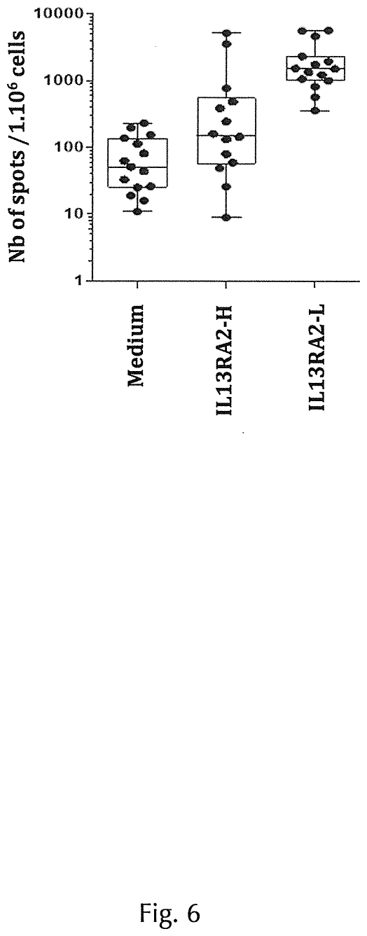

The number of spot forming cells (SFC) (normalized to the number of CD8 cells) are depicted in FIG. 6. Results are shown for mice immunized with IL13RA2-L. The results show that immunisation of mice with IL13RA2-L allows to induce T-cells that are able to react strongly after challenge with either IL13RA2-L or the human corresponding peptide. Thus, IL13RA2-L is strongly immunogenic and is able to drive an effective immune response against the corresponding human peptide. As expected, the immunisation of mice with the human corresponding peptide IL13RA2-H does not induce an immune response after challenge with either IL13RA2-L or the human corresponding peptide IL13RA2-H (data not shown).

These results were confirmed in HHD DR1 mice expressing human HLA-A2 and HLA-DR1 MHC and lacking the murine H-2 class I and class II MHCs (groups of 5 mice).

### Example 7: IL13RA2-L has Superior Affinity to the HLA-A*0201 Allele

This Example provides evidence that the (poly)peptide of the invention as set forth in SEQ ID NO: 279 (also referred to herein as IL13RA2-L) has a similarly high affinity to the HLA-A*0201 allele as the (poly)peptide of the invention as set forth in SEQ ID NO: 31 (FLPFGFILV, also referred to herein as IL13RA2-B)—and a higher affinity than the corresponding reference human peptide derived from IL13RA2 (IL13RA2-H, WLPFGFILI, SEQ ID NO: 263) and other sequence variants thereof. In this experiment, the (poly)peptide of sequence SEQ ID NO: 279 (IL13RA2-L) was compared to
the comparative peptide “1A9V”, as described by Eguchi Junichi et al., 2006, Identification of interleukin-13 receptor alpha 2 peptide analogues capable of inducing improved antiglioma CTL responses. Cancer Research 66(11): 5883-5891, in which the tryptophan at position 1 of SEQ ID NO: 263 was substituted by alanine (1A) and the isoleucine at position 9 of SEQ ID NO: 263 was substituted by valine (9V);
the (poly)peptide of the invention as set forth in SEQ ID NO: 31 (IL13RA2-B);
the corresponding reference human peptide IL13RA2-H (SEQ ID NO: 263); and
a positive control (HIV).

A. Materials and Methods

The experimental protocol, materials and methods correspond to those outlined in Example 2, with the only difference that the above mentioned (poly)peptides were used.

B. Results

The following in vitro binding affinities were obtained:

| | | Concentration of peptide | In vitro |
|---|---|---|---|
| | | that induces 20% of | binding |
| Peptide | SEQ ID NO | HLA-A2 expression (μM) | affinity |
| IL13RA2-H | 691 | ND | ND |
| IL13RA2-B | 254 | 2.9 | 0.3 |
| IL13RA2-L | 255 | 3.2 | 0.3 |
| 1A9V | 896 | 36.5 | 3.6 |

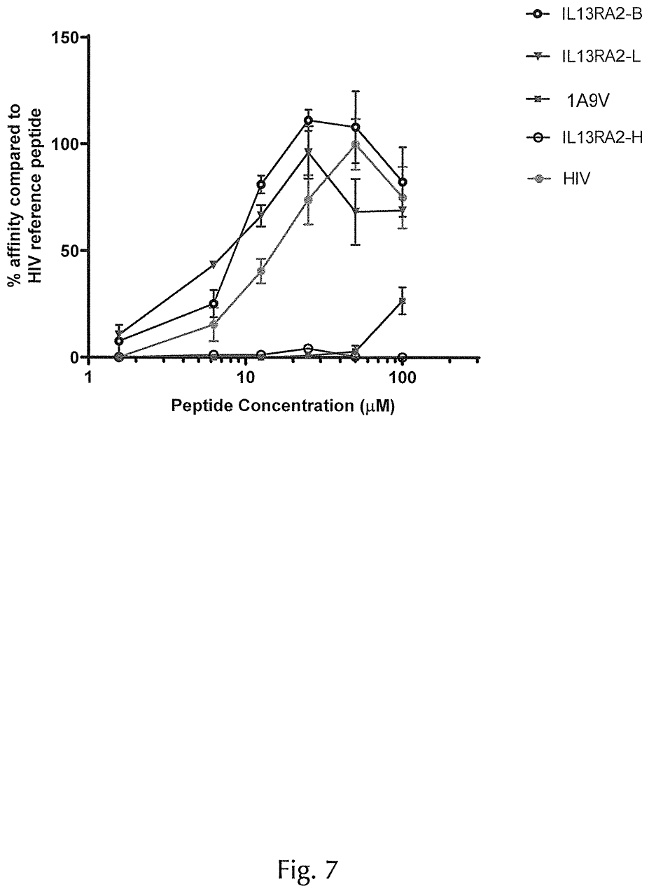

Accordingly, the (poly)peptides according to the present invention (IL13RA2-B; SEQ ID NO: 31 and IL13RA2-L; SEQ ID NO: 279) showed considerably higher binding affinity to HLA-A*0201 than the corresponding human peptide (IL13RA2-H) and the comparative peptide “1A9V”, as described by Eguchi Junichi et al., 2006, Identification of interleukin-13 receptor alpha 2 peptide analogues capable of inducing improved antiglioma CTL responses. Cancer Research 66(11): 5883-5891. In particular, the (poly)peptide IL13RA2-L (SEQ ID NO: 279) shows a strong binding affinity to HLA-A*0201, namely, 69% of maximum HIV pol 589-597 binding activity at 100 μM; 96% at 25 μM and 43% at 6.25 μM. Results are also shown in FIG. 7.

1.-59. (canceled)

60. A method for ameliorating, reducing and/or treating fibrosis, an autoimmune disease and/or an inflammatory disease in a subject comprising administering to the subject
(i) a (poly)peptide comprising an epitope of IL13RA2 or a sequence variant thereof having at least 70% sequence identity;
(ii) an immunogenic compound comprising the (poly)peptide as defined in (i);
(iii) a nanoparticle loaded with at least one (poly)peptide as defined in (i) or at least one immunogenic compound as defined in (ii);
(iv) a cell loaded with the (poly)peptide as defined in (i) or the immunogenic compound as defined in (ii);
(v) a nucleic acid encoding the (poly)peptide as defined in (i);
(vi) a host cell comprising the nucleic acid as defined in (v); or
(vii) a pharmaceutical composition comprising the (poly)peptide as defined in (i), an immunogenic compound as defined in (ii), a nanoparticle as defined in (iii), a cell as defined in (iv), a nucleic acid as defined in (v), or a host cell as defined in (vi).

61. The method according to claim 60, wherein the disease/disorder to be treated, ameliorated or reduced is fibrosis.

62. The method according to claim 61, wherein the fibrosis is selected from the group consisting of: a fibrosis of the lung, a fibrosis of the liver, a gastrointestinal fibrosis, a fibrosis of the heart, a fibrosis of the brain, a fibrosis of the skin, a fibrosis of a joint, a fibrosis of hands or feet, a fibrosis of the eye, a fibrosis of bone marrow and an allograft fibrosis.

63. The method according to claim 61, wherein the fibrosis is selected from the group consisting of nonalcoholic steatohepatitis (NASH), cirrhosis, biliary atresia, schistosomiasis-induced hepatic fibrosis, systemic sclerosis, idiopathic pulmonary fibrosis (IPF), cystic fibrosis, radiation-induced lung injury, proliferative vitreoretinopathy, fibrosis in chronic TNBS colitis, fibrosis in Crohn's disease, allograft fibrosis in organ transplantation, atrial fibrosis, endomyocardial fibrosis, old myocardial fibrosis, glial scar, arthrofibrosis, mediastinal fibrosis, myelofibrosis, nephrogenic systemic fibrosis, progressive massive fibrosis, and retroperitoneal fibrosis.

64. (canceled)

65. (canceled)

66. The method according to claim 60, wherein the disease/disorder to be treated, ameliorated or reduced is an autoimmune disease/disorder.

67. The method according to claim 60, wherein the disease/disorder to be treated, ameliorated or reduced is an inflammatory disease.

68. The method according to claim 66, wherein the disease/disorder to be treated, ameliorated or reduced is allergic inflammation.

69. The method according to claim 66, wherein the disease/disorder to be treated, ameliorated or reduced is asthma.

70. A method for eliciting an immune response against cells (over)expressing IL13RA2 in a subject comprising administering to the subject
(i) a (poly)peptide comprising an epitope of IL13RA2 or a sequence variant thereof having at least 70% sequence identity;
(ii) an immunogenic compound comprising the (poly)peptide as defined in (i);
(iii) a nanoparticle loaded with at least one (poly)peptide as defined in (i) or at least one immunogenic compound as defined in (ii);
(iv) a cell loaded with the (poly)peptide as defined in (i) or the immunogenic compound as defined in (ii);
(v) a nucleic acid encoding the (poly)peptide as defined in (i);
(vi) a host cell comprising the nucleic acid as defined in (v); or
(vii) a pharmaceutical composition comprising the (poly)peptide as defined in (i), an immunogenic compound as defined in (ii), a nanoparticle as defined in (iii), a cell as defined in (iv), a nucleic acid as defined in (v), or a host cell as defined in (vi).

71. The method according to claim 60, wherein the (poly)peptide does not bind to and/or inhibit IL-13.

72. The method according to claim 60, wherein the (poly)peptide has a maximum length of 350 amino acids.

73. The method according to claim 60, wherein the (poly)peptide comprises a sequence variant of an epitope of IL13RA2 having at least 70% sequence identity.

74. The method according to claim 60, wherein the core sequence of the sequence variant is identical with the core sequence of the IL13RA2 epitope, with the core sequence consisting of all amino acids except the three most N-terminal and the three most C-terminal amino acids.

75. The method according to claim 60, wherein the sequence variant is a microbiota sequence variant.

76. The method according to claim 75, wherein the microbiota sequence variant is a bacterial peptide.

77. The method according to claim 60, wherein the (poly)peptide comprises an amino acid sequence as set forth in any one of SEQ ID NOs 279, 192, 31, 1-30, 32-191, 193-242, and 267-274.

78. The method according to claim 60, wherein at least two distinct (poly)peptides are administered.

79. The method according to claim 78, wherein a (poly)peptide comprising an amino acid sequence as set forth in SEQ ID NO: 279 or 31 and a (poly)peptide comprising an amino acid sequence as set forth in SEQ ID NO: 192 are administered.

80. The method according to claim 60, wherein at least two distinct immunogenic compounds are administered.

81. The method according to claim 80, wherein an immunogenic compound comprising a (poly)peptide comprising an amino acid sequence as set forth in SEQ ID NO: 279 or 31 and an immunogenic compound comprising a (poly)peptide comprising an amino acid sequence as set forth in SEQ ID NO: 192 are administered.

82. The method according to claim 60, wherein at least two distinct nanoparticles are administered.

83. The method according to claim 82, wherein a nanoparticle loaded with a (poly)peptide comprising an amino acid sequence as set forth in SEQ ID NO: 279 or 31 and a nanoparticle loaded with a (poly)peptide comprising an amino acid sequence as set forth in SEQ ID NO: 192 are administered.

84. The method according to claim 60, wherein at least two distinct nucleic acids are administered.

85. The method according to claim 84, wherein a nucleic acid comprising a polynucleotide encoding an (poly)peptide having an amino acid sequence as set forth in SEQ ID NO: 279 or 31 and a nucleic acid comprising a polynucleotide encoding an (poly)peptide having an amino acid sequence as set forth in SEQ ID NO: 192 are administered.
